(19) United States
(12) Patent Application Publication
(10) Pub. No.: US 2026/0315664 A1
(43) Pub. Date: Oct. 15, 2026

(54) CO-POLYMERIC COMPOUNDS, HYDROGELS COMPRISING SAME AND USES THEREOF

(71) Applicant: Yeda Research and Development Co. Ltd., Rehovot (IL)

(72) Inventors: Jacob KLEIN, Rehovot (IL); Monika KLUZEK, Rehovot (IL); Weifeng LIN, Rehovot (IL); Panpan ZHAO, Rehovot (IL)

(73) Assignee: Yeda Research and Development Co. Ltd., Rehovot (IL)

(21) Appl. No.: 19/546,499

(22) Filed: Feb. 23, 2026

Related U.S. Application Data
(63) Continuation of application No. PCT/IL2024/050846, filed on Aug. 21, 2024.
(60) Provisional application No. 63/533,705, filed on Aug. 21, 2023.

(51) Int. Cl. A61L 31/10; A61L 31/16; C08F 220/36

(52) U.S. Cl. CPC A61L 31/10; A61L 31/16; C08F 220/36; A61L 2300/406

(57) ABSTRACT

A co-polymeric compound represented by Formula I:

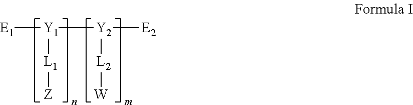

wherein the variables are described in the specification and claims, a composition comprising a hydrogel or a composite material containing a hydrogel what has incorporated therein the co-polymeric compound and a process of preparing such a composition, a method of lowering a friction coefficient of a hydrogel or of a composite material containing a hydrogel effected by forming the hydrogel in the presence of the co-polymeric compound, a method of inhibiting biofilm formation on a surface of a substrate comprising contacting the substrate with the composition or composite material, and an article-of-manufacturing comprising the composition or composite material, are provided.

### RELATED APPLICATIONS

This application is a Continuation of PCT Patent Application No. PCT/IL2024/050846 having International filing date of Aug. 21, 2024, which claims the benefit of priority under 35 USC § 119(e) of U.S. Provisional Patent Application No. 63/533,705 filed on Aug. 21, 2023. The contents of the above applications are all incorporated by reference as if fully set forth herein in their entirety.

### FIELD AND BACKGROUND OF THE INVENTION

The present invention, in some embodiments thereof, relates to material science, and more particularly, but not exclusively, to novel co-polymeric compounds usable, inter alia, in lubrication applications and/or in reducing biofilm formation on a surface of various substrates.

Natural articular cartilage is a thin layer of porous hydrated tissue with low friction that is unmatched by any artificial material [Jahn et al., Annual Review of Biomedical Engineering 2016, 18 (1), 235-258; Lin and Klein, Advanced Materials 2021, 33 (18), 2005513]. Damage caused by trauma or aging can lead to joint diseases, such as osteoarthritis [Lotz, M. K., Arthritis Research & Therapy 2010, 12 (3), 211; Loeser, R. F., Current Opinion in Rheumatology 2011, 23 (5), 492-496]. Hydrogels, which are widely used in biomedical and other applications [Green and Elisseeff, Nature 2016, 540 (7633), 386-394; Peppas et al., Advanced materials 2006, 18 (11), 1345-1360; Zhang et al., Angewandte Chemie International Edition 2022, 61 (39), e202209741], are considered suitable replacement materials for damaged articular cartilage due to their good biocompatibility, liquid permeability, and wettability [Ngadimin et al., Biomaterials Science 2021, 9 (12), 4246-4259; Zhou et al., Engineering 2022, 13, 71-90].

In recent years, several methods have been used to prepare lubricated hydrogels [Shoaib and Espinosa-Marzal, Colloids and Interfaces 2020, 4 (4), 54; Lin and Klein, Accounts of Materials Research 2022, 3 (2), 213-223; Zhang et al., Journal of the American Chemical Society 2018, 140 (9), 3186-3189; U.S. Pat. No. 6,800,298; Bavaresco et al., Wear, 2008, 265 (3-4), 269-277; Freeman et al. Wear, 2000, 241 (2), 129-135; Gong et al. Soft Matter, 2006, 2 (7), 544-552; WO 2015/193887].

Gulsen et al. [Current Eye Research, 2005, 30, 1071-1080] teach contact lens compositions with drug delivery capabilities, and specifically teach dispersing exceptionally small dimyristoylphosphatidylcholine (DMPC) SUV liposomes (less than 50 nm or 80 nm in diameter) in poly-2-hydroxyethyl methacrylate (pHEMA) hydrogels, which are common contact lens materials. The results of this study show that the pHEMA gels loaded with exceptionally small liposomes are transparent and release drugs for a period of about 8 days.

Recently, researchers have incorporated a biomimetic boundary lubricant poly(2-methacryloyloxyethylphosphorylcholine) (pMPC) polymer network into a high-strength poly(2-acrylamido-2-methylpropanesulfonic acid)-polyacrylamide double network (DN) gel, forming a three-component network (pMPC-DN) hydrogel [Milner et al. Acta Biomaterialia 2018, 65, 102-111]. Preliminary tribological studies showed that the friction coefficient of both DN and pMPC-DN hydrogels decreases with increasing sliding speed, which is consistent with two-phase lubrication in the physiological sliding velocity range, and that in the presence of the boundary-lubricated pMPC network, the friction coefficient of the pMPC-DN hydrogel is roughly half that of the DN hydrogel.

A bilayer hydrogel material inspired by the structure of the articular cartilage has also been designed, comprising thick hydrophilic polyelectrolyte brushes on the topmost of the hydrogel which provide effective water-based lubrication, while the hard hydrogel layer as the substrate provides load-bearing capacity [Rong et al., Advanced Functional Materials 2020, 30 (39), 2004062]. A synergistic effect has been reported therein, which was suggested to be due to high hydration of charged chains embedded in the composite hydrogel films, which enables a low friction coefficient under heavy load conditions in water, and thus allows its performance to be comparable to that of natural articular cartilage.

WO 2017/109784 discloses lipid-polymer conjugates, including lipid-pMPC conjugates, liposomes made therefrom and uses thereof. These liposomes have been reported as exhibiting improved stability and enhanced performance as lubricants compared to, for example, corresponding PEG-functionalized liposomes.

Fouling of surfaces by the adsorption of unwanted material from fluids can have harmful effects, such as posing health threats, promoting corrosion, and causing damage to industrial equipment. Biofilms, a group of microorganisms that stick to each other on a surface and produce a self-made matrix composed of extracellular DNA, proteins, and polysaccharides, can facilitate the formation of fouling by providing a surface for the adhesion of microorganisms. Biofilms can also accelerate the corrosion of metallic substrates and become highly antibiotic-resistant, leading to infections associated with medical devices in contact with human tissue, such as stents or catheters.

Infections resulting from biofilm formation on medical devices are common and can be costly, with the cost of curing such infections exceeding $50,000 per case. Catheters, in particular, are often colonized by bacteria, resulting in infections that represent about half of all hospital-associated infections (HAI) and are difficult to treat. Neutralizing bacteria in a biofilm may require a much higher dose of antibiotics compared to planktonic bacteria, and infections associated with medical devices typically begin with the adhesion of bacteria to a surface, followed by their proliferation, resulting in a biofilm formation.

Efforts have been made to develop materials which exhibit reduced protein adhesion, with the goal of reducing adhesion of microbes that form biofilms, as microbial adhesion is mediated by surface proteins.

Hydrogels formed by copolymerization of carboxybetaine monomers with 2-hydroxyethyl methacrylate (HEMA) have been reported to exhibit low levels of fouling from blood plasma [Kostina et al., Biomacromolecules 2012, 13, 4164-4170]. Modification of polysulfone (PSf) membranes with 2-methacryloxyethyl phosphorylcholine (MPC) polymer was reported to decrease protein adsorption [Ishihara et al., Biomaterials 1999, 20, 1553-1559]. Other studies have associated zwitterionic hydrogel surfaces with enhanced antifouling properties [Schlenoff, Langmuir 2014, 30, 9625-9636; Yang et al., J Mat Chem B 2014, 2, 577-584].

Contact lens frequently adsorb proteins from the eye fluid, which limits their long-term use. The accumulated protein can lead to discomfort and eye infection, as bacterial adhesion to soft contact lenses and biofilm formation have been reported to be associated with keratitis [Bourcier et al., Br J Ophthalmol 2003, 87:834-838; Tam et al., Invest Ophthalmol Vis Sci 2010, 51, 3100-3106].

WO 2015/001564 describes hydrogels having liposomes dispersed therein, which exhibit a reduced friction coefficient compared to neat hydrogels, processes for preparing the same, and methods for using the same, e.g., in replacing missing or damaged cartilage in a living organism suffering from a medical condition associated with loss of or damaged cartilage.

WO 2015/193888 discloses solutions comprising a water-soluble polymer, liposomes, and an aqueous carrier, for reducing a friction coefficient of a surface, and methods utilizing same.

Additional background art includes Goldberg et al., Advanced Materials 2011, 23 (31), 3517-3521; Seror et al., Nature communications 2015, 6 (1), 1-7; Schmidt, T. A., Science 2020, 370 (6514), 288-289; Lin et al. Science 2020, 370 (6514), 335-338; Nicolson and Vogt, Biomaterials 2001, 22 (24), 3273-3283; Ishihara, K., Polymer Journal 2015, 47 (9), 585-597; Chen et al. Science 2009, 323 (5922), 1698-1701; Mizrahi et al., Langmuir 2013, 29:10087-10094; Bozukova et al., Langmuir 2008, 24, 6649-6658; WO 2008/038292; U.S. Patent Application Publication No. 2010/0098749; WO 2016/051413; WO 2017/109784; Matsuda et al. Polymer Journal 2008, 40 (5), 479-483; and Xie et al. Nature Biomedical Engineering 2021, 5 (10), 1189-1201.

### SUMMARY OF THE INVENTION

According to an aspect of some embodiments of the invention, there is provided a co-polymeric compound represented by Formula I:

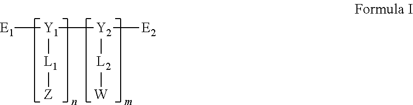

wherein:
m and n are each independently a positive integer;
Y₁ and Y₂ are each independently a backbone unit which forms a polymeric backbone;
L₁ and L₂ are each independently absent or is a linking moiety;
E₁ and E₂ each independently represents a terminal group;
W is a hydrophobic group; and
Z is represented by Formula II:

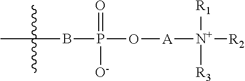

wherein:
A is a substituted or unsubstituted hydrocarbon;
B is an oxygen atom or is absent; and
R₁-R₃ are each independently selected from the group consisting of hydrogen, alkyl, cycloalkyl, heteroalicyclic, aryl and heteroaryl,
wherein when n+m is greater than 2, the —[Y₁-L₁-Z]— units and the —[Y₂-L₂-W]— units are arranged in any order so as to form a random co-polymeric compound.

According to some of any of the embodiments described herein, Y₁ and Y₂ are each independently a substituted or unsubstituted alkylene unit.

In some of any of the embodiments described herein, Y₁ and Y₂ are each independently a substituted or unsubstituted ethylene unit.

In some of any of the embodiments described herein, each of Y₁ and Y₂ independently has the formula —CR₄R₅—CR₆D-, wherein:
when Y₁ is a backbone unit which is not attached to the L₁ or the Z, D is R₇; and when Y₁ is a backbone unit which is attached to the L₁ or the Z, D is absent or a linking group attaching Y₁ to the L₁ or the Z,
and when Y₂ is a backbone unit which is not attached to the L₂ or the W, D is R₈; and when Y₂ is a backbone unit which is attached to the L₂ or the W, D is absent or a linking group attaching Y₂ to the L₂ or the W,
wherein when D is a linking group, the linking group being selected from the group consisting of —O—, —S—, alkylene, arylene, sulfinyl, sulfonyl, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, and amino; and
R₄-R₈ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azide, azo, phosphate, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, and amino.

In some of any of the embodiments described herein, R₄ and R₅ are each hydrogen.

In some of any of the embodiments described herein, R₆ is hydrogen or alkyl (e.g., methyl).

According to some of any of the embodiments described herein, when D is a linking group, the linking group is selected from the group consisting of —O—, —C(═O)O—, —C(═O)NH— and phenylene.

In some of any of the embodiments described herein, the linking group is —C(═O)NH—.

In some of any of the embodiments described herein, L₁ is a substituted or unsubstituted hydrocarbon from 1 to 10 carbon atoms in length.

In some of any of the embodiments described herein, L₁ is a substituted or unsubstituted ethylene group.

According to some of any of the embodiments described herein, B is an oxygen atom.

According to some of any of the embodiments described herein, A is a substituted or unsubstituted hydrocarbon of from 1 to 4 carbon atoms in length.

In some of any of the embodiments described herein, A is a substituted or unsubstituted ethylene group.

According to some of any of the embodiments described herein, R₁-R₃ are each independently hydrogen or C₁₋₄-alkyl.

In some of any of the embodiments described herein, R₁-R₃ are each methyl.

According to some of any of the embodiments described herein, E₁ is or comprises a lipid moiety.

According to some of any of the embodiments described herein, the sum of the n and the m (e.g., n+m) is at least 3.

According to some of any of the embodiments described herein, the sum of n+m is in a range of from 3 to 5000.

In some of any of the embodiments described herein, a ratio of n to m, representing a mol ratio between the —[Y₁-L₁-Z]— units and the —[Y₂-L₂-W]— units in the co-polymeric compound, is in a range of from 1:10 to 1:50, or from 1:2 to 1:50, or from 1:3 to 1:10 (n: m).

According to some of any of the embodiments described herein, the polymeric compound comprises at least one targeting moiety. According to some of any of the embodiments described herein, the targeting moiety is attached to one or more of the Y₁ and Y₂ backbone units, E₁ and E₂ terminal groups, L₁ and L₂ linking moieties, the Z group and the W group.

According to some of any of the embodiments described herein, W is a hydrocarbon of at least 3 carbon atoms in length.

According to some of any of the embodiments described herein, W is an alkyl of at least 3 carbon atoms in length.

According to some of any of the embodiments described herein, W is or comprises a branched alkyl of at least 3 carbon atoms in length.

According to some of any of the embodiments described herein, W is isopropyl.

According to an aspect of some embodiments of the invention, there is provided a composition comprising a hydrogel or a composite material comprising a hydrogel, the hydrogel having incorporated therein the co-polymeric compound according to some of any of the embodiments described herein.

According to some of any of the embodiments described herein, an amount of the co-polymeric compound is in a range of from 0.1% to 10%, or from 0.1% to 5%, by weight, of the total weight of the composition or of the hydrogel or the composite material.

According to some of any of the embodiments described herein, a degree of crosslinking of the hydrogel is in a range of from 1 to 10, or from 1 to 5, %.

According to some of any of the embodiments described herein, the hydrogel is formed of at least one hydrogel-forming agent selected from hydroxyethyl methacrylate (HEMA), hydroxyethyl acrylate (HEA), acrylamide (AAm), methacrylamide (MAAm), acrylic acid (AAc), methacrylic acid (MAAc), hydroxyethyl acrylate (HEA), hexyl methacrylate, N-isopropylacrylamide (NIPAM)), N-isopropylmethacrylamide, polylactic acid, polyamide, polyethylene-terephthalate (PET), polyvinyl alcohol, polyurethane, polycaprolactone, polyethylene-glycol (PEG), polyethylene-glycol methacrylate (PEGMA), polyethyleneoxide dimethacrylate (PEOdMA), N,N-dimethacrylamide (nnDMAA), hyaluronic acid (HA), HA methacrylate, peptides, saccharides, gelatin, gelatin methacrylate, chitosan, chitosan methacrylate, glycol chitosan, glycol chitosan methacrylate, alginate, alginate methacrylate, cellulose, siloxanes, polysiloxanes, and any oligomer, polymer and/or copolymer thereof, in any combination thereof.

According to some of any of the embodiments described herein, the hydrogel comprises hydroxyethyl methacrylate (HEMA) and/or poly hydroxyethyl methacrylate (pHEMA).

According to some of any of the embodiments described herein, the composition or composite material is in a dry form.

According to some of any of the embodiments described herein, the composition of composite material is characterized by at least one of:
storage modulus G′ (at 1 Hz) of at least 10 MPa;
loss modulus G″ (at 1 Hz) of at least 1,000 Pa; and
sliding friction coefficient μ lower than 0.05, or lower than 0.01. According to some of these embodiments, the friction is versus a polyethylene (hydrophobic) sphere.

According to some of any of the embodiments described herein, the composition or composite material further comprises at least one additional agent selected from the group consisting of a polymeric compound, a hydrogel-forming agent, a sterol (e.g., cholesterol), a liposome-stabilizing agent, a labeling agent, a bioactive agent, and a therapeutically active agent.

According to an aspect of some embodiments of the invention, there is provided a process for preparing the composition or composite according to any of the embodiments described herein, the process comprising: contacting an aqueous solution or suspension comprising a hydrogel-forming agent as described herein in any of the respective embodiments and any combination thereof and a crosslinking agent with the co-polymeric compound as described herein in any of the respective embodiments and any combination thereof, thereby obtaining the composition.

In some of any of the embodiments described herein, the aqueous solution or suspension further comprises a catalyst.

According to some of any of the embodiments described herein, the process further comprises dehydrating the composition.

In some of any of the embodiments described herein, the process further comprises, subsequent to the dehydrating, rehydrating the composition.

According to some of any of the embodiments described herein, the crosslinking agent is selected from the group consisting of poly(ethylene glycol) n dimethacrylate (EGDMA), N,N′-methylenebis(acrylamide) (MBAm), N,N′-methylenebis(2-methylacrylamide), methylene diacrylate, methylene bis(2-methylacrylate), diethylene glycol diacrylate, hexamethylene diacrylate, oxybis(methylene)bis(2-methylacrylate) and oxybis(ethane-2,1-diyl) bis(2-methylacrylate).

According to an aspect of some embodiments of the invention, there is provided a composition or composite material prepared by the process according to some of any of the embodiments described herein.

According to some of any of the embodiments described herein, the composite material comprises a material selected from the group consisting of a woven mesh of fibers, non-woven fibers, a plurality of rods and a net.

According to an aspect of some embodiments of the invention, there is provided a method of lowering a friction coefficient of a hydrogel or of a composite material comprising a hydrogel, the method comprising forming the hydrogel in the presence of the co-polymeric compound as described herein in any of the respective embodiments and any combination thereof.

According to some of any of the embodiments described herein, the dynamic friction coefficient in aqueous medium of the hydrogel or composite material containing a hydrogel having the co-polymeric compound incorporated therein is reduced by a factor of at least 2 relative to the friction coefficient of the hydrogel not having the co-polymeric compound incorporated therein.

According to some of any of the embodiments described herein, the method further comprises dehydrating the hydrogel or composite material containing a hydrogel and rehydrating the hydrogel.

According to some of any of the embodiments described herein, the friction coefficient is substantially maintained after at least one dehydration-rehydration cycle.

According to an aspect of some embodiments of the invention, there is provided an article-of-manufacturing comprising the composition or composite material as described herein in any of the respective embodiments and any combination thereof.

According to some of any of the embodiments described herein, the article-of-manufacturing is selected from the group consisting of an implantable medical device, a drug-delivery system, a solid body, a disc, a fiber, a fabric, a tube, a film, a rod, a ring, a tubular mesh and any combination thereof.

According to some of any of the embodiments described herein, the composition or composite material is for use in replacing missing or damaged cartilage in a living organism suffering from a medical condition associated with loss of or damaged cartilage.

According to some of any of the embodiments described herein, the medical condition is selected from the group consisting of a skeletal joint replacement or reconstruction, vertebrate replacement or reconstruction, tendon replacement, tissue regeneration and reduction of tissue irritation by an implantable device.

According to an aspect of some embodiments of the invention, there is provided a method of lowering the friction coefficient of a surface of a substrate, the method comprising applying to the surface the composition or composite material according to any of the embodiments described herein.

According to some of any of the embodiments described herein, lowering the friction coefficient is under aqueous or non-aqueous conditions.

According to some of any of the embodiments described herein, the substrate is a physiological substrate.

According to some of any of the embodiments described herein, the substrate is a medical device, e.g., an implantable medical device.

According to an aspect of some embodiments of the invention, there is provided a method of inhibiting biofilm formation on a surface of a substrate, the method comprising contacting the substrate with a composition or composite material according to some of any of the embodiments described herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B present the chemical structure of an exemplary co-polymeric compound according to some embodiments of the present invention, poly(2-methacryloyloxyethyl phosphorylcholine-co-N-isopropylacrylamide) (PMN) co-polymer (FIG. 1A) and a relevant portion of its ¹H-NMR spectrum in D₂O (FIG. 1B).

FIGS. 2A-B are cryo-SEM images presenting characterization of PMN-free (FIG. 2A) and PMN-incorporating (FIG. 2B) pHEMA hydrogels.

FIGS. 2C-D are confocal microscopy images showing an orthogonal view (FIG. 2C, upper inset) and an in-plane view (FIG. 2C, rightmost inset, and FIG. 2D) of a 40 μm thick slice hydrogel incorporating fluorescently labeled PMN pockets recorded at room temperature (RT). Scale bars are 20 μm in FIG. 2C and 10 μm in FIG. 2D.

FIGS. 3A-B present comparative plots showing size distribution (FIG. 3A) and zeta potential (FIG. 3B) characterizations of pHEMA (red) and pHEMA/PMN mixture (green) in water, as measured by DLS after dialysis, as described herein.

FIGS. 4A-C present photographs (FIGS. 4A-B, acquired as described herein) of rhodamine labeled PMN-pHEMA hydrogel (prepared as described herein) at T₁=0 hour (FIG. 4A) and T₂=442 hours (FIG. 4B); and a plot showing the kinetic profile of PMN-rhodamine-conjugate released from the rhodamine labeled PMN-pHEMA hydrogel (0.5 cm or 0.8 cm thickness, diameter: 1.6 cm) (FIG. 4C) upon incubation at physiological conditions (PBS, 37° C.) as described herein in the Method section (see, In-vitro PMN release studies).

FIGS. 5A-D are images of Brightfield (FIGS. 5A and 5C) and Syto9 (FIGS. 5B and 5D) fluorescence confocal microscopy images of neat (FIGS. 5A and 5B) and PMN-incorporating (FIGS. 5C and 5D) pHEMA hydrogels. Scale bar is 20 μm.

FIG. 6 presents comparative plots showing the storage and loss moduli (G′ and G″, respectively) of PMN-free and PMN-incorporating (2%) pHEMA hydrogels.

FIGS. 7A-E are bar graphs showing sliding friction coefficients (μ): of 0.1%, 0.3% and 1.0% PMN-incorporating pHEMA hydrogels relative to the PMN-free HEMA hydrogel at different loads, between a pHEMA hydrogel and a sliding stainless steel sphere (hydrophilic) (FIG. 7A); of different PMN-free (denoted as “neat”) hydrogels (HEMA-co-HEAA, HEMA-co-DMAA, PEGMA) and a stainless-steel sphere, relative to the respective 1% PMN-incorporating hydrogel (FIG. 7B); of neat pHEMA and different polymer-incorporating (1% PMN, 1% pNIPAM, 0.5% pMPC, or a combination of 1% pNIPAM and 0.5% pMPC) pHEMA hydrogels at different loads (10.0 N and 30.0 N) between a hydrogel and a sliding steel sphere (FIG. 7C); of neat and different lubricant-incorporating (1% DMPC-incorporating or 1% PMN-incorporating) pHEMA hydrogels at different loads (10.0 N and 30.0 N) between a pHEMA hydrogel and a polyethylene sphere (hydrophobic) (FIG. 7D); and of 1% PMN-incorporating pHEMA hydrogel before and after dehydration and rehydration, as measured against stainless steel sphere (FIG. 7E).

FIG. 7F presents comparative plots showing sliding friction coefficients (μ) of PMN-free and PMN-incorporating pHEMA hydrogels at different sliding velocity (Vs), as measured against stainless steel sphere. Error bars indicate standard deviation (SD) from at least three measurements.

FIGS. 8A-C are photographs (acquired as described herein) of PMN-free pHEMA hydrogel (FIG. 8A) and pHEMA hydrogels incorporating DMPC (lipid) (FIG. 8B) and PMN copolymer (FIG. 8C) samples on a polyethylene (PE) sphere after 1 hour of sliding back-and-forth under a 10 N load. Hydrogels are immersed in 3 mL of water.

FIGS. 9A-D present fluorescence signals recorded by Typhoon FLA 9500 fluorescence scanner (GE Healthcare Bio-Sciences AB, Sweden) on polyethylene (PE) (FIGS. 9A and 9C) and stainless-steel (SS) (FIGS. 9B and 9D) spherical ball upon 5 minutes sliding at 1 mm/s velocity on rhodamine-labeled PMN (FIGS. 9A and 9B) and DiI-labelled DMPC liposomes (FIGS. 9C and 9D) incorporated in pHEMA hydrogels. Photomultiplier tube set up at 500′ gain and pixel size 50 μm. Scale bar is 0.2 cm.

FIGS. 10A-B present comparative plots showing the fluorescence signal of PMN-rhodamine (FIG. 10A) and DMPC-DiI (FIG. 10B) obtained from stainless steel (SS) and polyethylene (PE) heads upon 5 minutes sliding. The insets present the calibration curve of rhodamine-PMN in water (FIG. 10A) and DMPC-DiI in chloroform (FIG. 10B) solutions.

FIGS. 11A-B are bar graphs showing sliding friction coefficients (μ) at 100 grams load of a commercial urethral PE catheter (lubricated with glycerol, or modified without lubricant, with neat pHEMA hydrogel, or with PMN-incorporating pHEMA hydrogel) against a sliding polyethylene (hydrophobic) (FIG. 11A), or a sliding pHEMA sphere (FIG. 11B). PMN concentration in the hydrogel was 2%, and the pHEMA hydrogel was 2% cross-linked.

FIGS. 12A-C present a schematic illustration of a Vero cells culture setup for evaluating the cytotoxic effect of PMN-incorporating pHEMA hydrogel (1%. PMN) (FIG. 12A); photographs of neat and PMN-incorporating pHEMA hydrogels placed in Transwell® (FIG. 12B); and a bar graph showing cellular viability of the cells following 48 hours incubation with and without the pHEMA hydrogels (FIG. 12C). Data represents two biological repeats (black and white data point) with a minimum of two technical repeats each.

FIG. 13A presents representative Typhoon-scanned fluorescence images of neat pHEMA hydrogels and of pHEMA hydrogels incorporating DMPC or PMN, incubated for 24 hours with Vero E6 cells labeled with Vybrant DiD (denoted “DiD”) or incubated for 12 hours with GFP-expressing E. coli bacteria (denoted “GFP”). Scale bar is 1.5 mm.

FIGS. 13B-C present quantification of the DiD fluorescence intensity (FIG. 13B) and GFP fluorescence intensity (FIG. 13C) of pHEMA hydrogels obtained from plate reader measurements. Fluorescence intensity measurements were conducted on 4 biological samples. Boxes represent the 25-75 percentiles of the sample distribution, with black vertical lines representing 1.5×IQR (interquartile range). Black horizontal line represents the median.

FIGS. 14A-B present confocal microscopy images of neat, DMPC-incorporating and PMN-incorporating pHEMA hydrogels after 24 hours incubation with Vero E6 cells labeled with Vybrant DiD (FIG. 14A), and after 12 hours incubation with GFP-expressing E. coli bacteria (FIG. 14B). Images are presented as respective fluorescence signals (yellow-DiD, and green-GFP) and overlays of brightfield and fluorescence staining. Scale bars are 20 μm.

FIG. 15A presents a chemical structure and the relevant portion of its ¹H-NMR spectrum in methanol-d₄ of the co-polymeric compound poly(2-methacryloyloxyethyl phosphorylcholine-co-N-propylacrylamide) (PMP).

FIG. 15B presents a comparative bar plot showing variation of the coefficient of sliding friction (COF (μ)) between a smooth PE sphere and pHEMA hydrogels incorporating 1% by weight of either PMN or PMP co-polymers under a load of Fn=10 N. COF was measured by calculating Fs/Fn. The inset presents traces of the shear force Fs transmitted between the PE surface and the pHEMA hydrogels incorporating the PMN or PMP co-polymers; the upper (zig-zag) trace is the applied lateral motion, and the two bottom traces are the transmitted shear forces, Fs. PMP co-polymer data is based on 6 polyethylene/hydrogel contact points in two independent experiments, i.e., separately-prepared PMP-incorporating hydrogels.

FIG. 15C presents comparative plots showing the storage and loss moduli (G′ and G″, respectively) of PMP- and PMN-incorporating (1% by weight) pHEMA hydrogels.

### DESCRIPTION OF SPECIFIC EMBODIMENTS OF THE INVENTION

The present invention, in some embodiments thereof, relates to material science, and more particularly, but not exclusively, to novel co-polymeric compounds usable, inter alia, for lubrication applications and/or for reducing biofilm formation on a surface of various substrates.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

In a search for polymeric materials that can be beneficially utilized as lubricants in a wide-spectrum of applications, the present inventors have turned to design such polymeric materials that feature both a plurality of positively-charged groups and a plurality of hydrophobic groups.

The present inventors have designed and successfully prepared and practiced an exemplary such co-polymeric compound, a poly(2-methacryloyloxyethyl phosphorylcholine-co-N-isopropylacrylamide) (pMPC-co-pNIPAM, PMN) random copolymer, which features highly-hydrated lubricous 2-methacryloyloxyethyl phosphorylcholine moieties and less hydrated N-isopropylacrylamide moieties.

As shown in the Examples section that follows, the incorporation of exemplary PMN copolymers into various hydrogels significantly reduced the hydrogels' sliding surface friction, resulting in low friction coefficients against different counter surfaces, including stainless steel (hard metal surface), polyethylene (hydrophobic surface), and polyHEMA (pHEMA; soft hydrogel surface). Additionally, PMN-incorporating hydrogels were shown to be biocompatible and have excellent antifouling properties, making them an attractive component in, e.g., coating of commercially available stents.

As further shown in the Examples section that follows, the exemplary PMN co-polymer, which features a branched alkyl, exhibits a superior lubricating performance compared to a similar co-polymer that features a linear alkyl having the same number of carbon atoms.

Altogether, PMN-incorporating hydrogels represent new materials which are beneficial for numerous possible applications.

### Co-Polymeric Compound:

According to an aspect of some embodiments of the invention, there is provided a co-polymeric compound represented by Formula I:

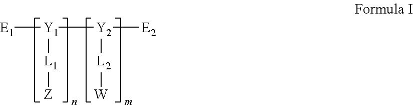

wherein:
m and n are each independently a positive integer;
Y₁ and Y₂ are each independently a backbone unit which forms a polymeric backbone;
L₁ and L₂ are each independently absent or is a linking moiety;
E₁ and E₂ each independently represents a terminal group, as described herein;
W is a hydrophobic group; and
Z is represented by Formula II:

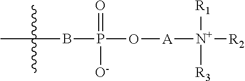

wherein:
A is a substituted or unsubstituted hydrocarbon;
B is an oxygen atom or is absent; and

R₁-R₃ are each independently selected from the group consisting of hydrogen, alkyl, cycloalkyl, heteroalicyclic, aryl and heteroaryl,
wherein when n+m is greater than 2, the —[Y₁-L₁-Z]— units and the —[Y₂-L₂-W]— units are arranged in any order so as to form a random co-polymeric compound,
as described in more detail herein below.

Formula I may also be described herein simply as:

E₁-[Y₁(-L₁-Z)]n[Y₂(-L₂-W)]m-E₂
which is to be regarded as interchangeable with the schematic depiction hereinabove.

As used herein, the term “backbone unit” refers to a repeating unit, wherein linkage between a plurality of repeating units (e.g., sequential linkage) forms a polymeric backbone. The plurality of repeating units per se (regardless of the pendant groups) when linked to one another, is also referred to herein as a “polymeric backbone”. A plurality

Herein, the term “polymeric compound” refers to a compound having at least 2 repeating units (and more preferably at least 3 repeating units, and up to 50,000 repeating units). When all the repeating units are the same, a polymeric compound is considered a homopolymeric compound. When the polymeric compound comprises two or more types of repeating units, which can differ from one another by the type of the backbone units that form the polymeric backbone and/or by the type of the pendant groups, the polymeric compound is a co-polymeric compound.

Herein, the term “co-polymeric compound” refers to a polymeric compound comprising at least two different types of monomeric units. It is to be appreciated that the compound represented by Formula I is by definition co-polymeric when n and m are each a positive integer, as it comprises at least two backbone units represented by Y₁ and Y₂, respectively. In exemplary embodiments, n is 200 and m is 1400.

In any of the embodiments described herein, the co-polymeric compound comprises two or more different types of monomeric unit which may be distributed randomly or non-randomly throughout the polymeric moiety. When different types of monomeric units are distributed non-randomly, the copolymer may be characterized by any non-random distribution, and can be, for example, an alternating copolymer, a periodic copolymer, and/or a block copolymer.

As used herein, the phrase “alternating copolymer” describes a copolymer in which two adjacent backbone units alternate in a regular sequence along the polymer chain, such that the polymer has a repeating pattern of the two distinct backbone units. Typically, in an alternating copolymer, each two adjacent backbone units are different from one another (e.g., Y1-Y2-Y1-Y2 or Y1-Y2-Y3-Y1-Y2-Y3)

As used herein, the phrase “periodic copolymer” describes a copolymer in which the arrangement of monomeric units follows a repeating sequence with a defined periodicity, which may involve more than two types of monomers, creating a regular and predictable sequence along the polymer chain (e.g., Y1-Y2-Y2-Y1-Y2-Y2-Y1-Y2-Y2-).

As used herein, the phrase “block copolymer” describes a copolymer in which distinct blocks of repeating backbone units are linked together in segments, such that the polymer consists of large sequences (“blocks”) composed of a plurality (e.g., 3 or more) of one type of backbone unit followed by a block of a plurality of another type of backbone unit (e.g., Y1-Y1-Y1-Y2-Y2-Y2).

In some of any of the embodiments described herein, the co-polymeric compound is a random co-polymeric compound in which the different types of the repeating backbone units, are distributed randomly, in any order, throughout the co-polymeric moiety.

Herein, the phrase “co-polymeric moiety” refers to the portion of the co-polymeric compound (according to any of the embodiments described herein relating to Formula I) which is represented by Formula Ia:

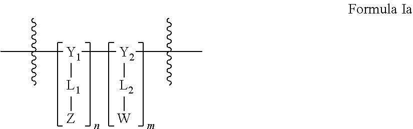

wherein m, n, Y₁, Y₂, L₁, L₂, Z and W are as defined herein for Formula I.

Formula Ia may also be described herein simply as:

—[Y₁(-L₁-Z)]n[Y₂(-L₂-W)]m-
which is to be regarded as interchangeable with the schematic depiction hereinabove.

As shown in Formulae I and Ia, L₁ and Z together and L₂ and W together each form a pendant group of at least a portion of the backbone units (i.e., Y₁ and Y₂, respectively). Each of these groups is referred to herein for brevity simply as the “pendant group”.

Each backbone unit Y₁ with its respective pendant group (i.e., a unit represented by Y₁(-L₁-Z), the number of which is represented by the variable n) and each backbone unit Y₂ with its respective pendant group (a unit represented by Y₂(-L₂-W), the number of which is represented by the variable m) is also referred to herein as a “monomeric unit”.

A backbone unit may optionally be a residue of a polymerizable monomer or polymerizable moiety of a monomer. A wide variety of polymerizable monomers and moieties are known to the skilled person, and the structure of the residues of such monomers which result upon polymerization (e.g., monomeric units) are also known to the skilled person.

A “residue of a polymerizable monomer” refers to a modified form of a polymerizable monomer and/or a portion of a polymerizable monomer that remains after polymerization.

A portion of a polymerizable monomer may be formed, for example, by a condensation reaction, e.g., wherein at least one atom or group (e.g., a hydrogen atom or hydroxyl group) in the monomer, and optionally at least two atoms or groups (e.g., a hydrogen atom and a hydroxyl group) in the monomer, is replaced with a covalent bond with another polymerizable monomer.

A modified form of a polymerizable monomer may be formed, for example, by ring-opening (wherein a covalent bond between two atoms in a ring is broken, and the two atoms optionally each become linked to another polymerizable monomer); and/or by adding to an unsaturated bond, wherein an unsaturated bond between two adjacent atoms is broken (e.g., conversion of an unsaturated double bond to a saturated bond, or conversion of an unsaturated triple bond to an unsaturated double bond) and the two atoms optionally each become linked to another polymerizable monomer.

A modified form of a polymerizable monomer may consist essentially of the same atoms as the original monomer, for example, different merely in the rearrangement of covalent bonds, or alternatively, may have a different atomic composition, for example, wherein polymerization includes a condensation reaction (e.g., as described herein).

Examples of backbone units include, without limitation, substituted or unsubstituted hydrocarbons (which may form a substituted or unsubstituted hydrocarbon backbone), such as alkylene units; hydroxycarboxylic acid units (which may form a polyester backbone), e.g., glycolate, lactate, hydroxybutyrate, hydroxyvalerate, hydroxycaproate and hydroxybenzoate units; dicarboxylic acid units (which may form a polyester backbone in combination with a diol and/or a polyamide in combination with a diamine), e.g., adipate, succinate, terephthalate and naphthalene dicarboxylic acid units; diol units (which may form a polyether backbone, or form a polyester backbone in combination with a dicarboxylic acid), e.g., ethylene glycol, 1,2-propanediol, 1,3-propanediol, 1,4-butanediol, and bisphenol A units; diamine units (which may form a polyamide backbone in combination with a dicarboxylic acid), e.g., para-phenylene diamine and alkylene diamines such hexylene diamine; carbamate units (which may form a polyurethane backbone); amino acid residues (which may form a polypeptide backbone); and saccharide moieties (which may form a polysaccharide backbone).

In some embodiments of any of the embodiments described herein, Y₁ and Y₂ are each independently a substituted or unsubstituted alkylene unit.

In some embodiments of any of the embodiments described herein, Y₁ and Y₂ are each independently a substituted or unsubstituted ethylene unit, that is, an alkylene unit 2 atoms in length.

Polymeric backbone units wherein Y₁ and Y₂ are each independently a substituted or unsubstituted ethylene unit may optionally be a polymeric backbone such as formed by polymerizing ethylene (CH₂═CH₂) and/or substituted derivatives thereof (also referred to herein as “vinyl monomers”). Such polymerization is a very well-studied procedure, and one of ordinary skill in the art will be aware of numerous techniques for effecting such polymerization.

It is to be understood that any embodiments described herein relating to a polymeric backbone formed by a polymerization encompass any polymeric backbone having a structure which can be formed by such polymerization, regardless of whether the polymeric backbone was formed in practice by such polymerization (or any other type of polymerization).

As is well known in the art, the unsaturated bond of ethylene and substituted ethylene derivatives becomes saturated upon polymerization, such that the backbone units in a polymeric backbone are saturated, although they may be referred to as units of an unsaturated compound (e.g., a “vinyl monomer” or “olefin monomer”) to which they are analogous.

Polymers which can be formed from unsaturated monomers such as vinyl monomers and olefin monomers are also referred to by the terms “polyvinyl” and “polyolefin”.

Herein, an “unsubstituted” alkylene unit (e.g., ethylene unit) refers to an alkylene unit which does not have any substituent other than the pendant groups discussed herein (represented as (-L₁-Z) and (-L₂-W)). That is, an alkylene unit attached to the aforementioned pendant groups is considered unsubstituted if there are no substituents at any other positions on the alkylene unit.

In some embodiments of any of the embodiments described herein, each of Y₁ and Y₂ independently has the formula —CR₄R₅—CR₆D-, wherein:
when Y₁ is a backbone unit which is not attached to the L₁ or the Z, D is R₇; and when Y₁ is a backbone unit which is attached to the L₁ or the Z, D is absent or a linking group attaching Y₁ to the L₁ or the Z,
and when Y₂ is a backbone unit which is not attached to the L₂ or the W, D is R₈; and when Y₂ is a backbone unit which is attached to the L₂ or the W, D is absent or a linking group attaching Y₂ to the L₂ or the W,
wherein when D is a linking group, the linking group being selected from the group consisting of —O—, —S—, alkylene, arylene, sulfinyl, sulfonyl, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, and amino; and
R₄-R₈ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azide, azo, phosphate, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, and amino.

Herein, the phrase “linking group” describes a group (e.g., a substituent) that is attached to two or more moieties in the compound.

Herein, the phrase “end group” describes a group (e.g., a substituent) that is attached to a single moiety in the compound via one atom thereof.

In some embodiments of any of the embodiments described herein, R₄ and R₅ are each hydrogen. Such embodiments include polymeric backbones formed from many widely used vinyl monomers (including ethylene), including, for example, olefins (e.g., ethylene, propylene, 1-butylene, isobutylene, 4-methyl-1-pentene), vinyl chloride, styrene, vinyl acetate, acrylonitrile, acrylate and derivatives thereof (e.g., acrylate esters, acrylamides), and methacrylate and derivatives thereof (e.g., methacrylate esters, methacrylamides).

In some embodiments of any of the embodiments described herein, R₆ is hydrogen. In some such embodiments, R₄ and R₅ are each hydrogen. When each of R₄-R₆ is hydrogen, and D is a linking group attaching Y₁ to the L₁ or the Z, or attaching Y₂ to the L₂ or the W, Y₁ and Y₂ each independently is an unsubstituted ethylene group attached (via D) to a pendant group as described herein.

In some embodiments of any of the embodiments described herein, R₆ is methyl. In some such embodiments, R₄ and R₅ are each hydrogen. In some such embodiments, the backbone unit is a unit of methacrylate or a derivative thereof (e.g., methacrylate ester, methacrylamide).

In some embodiments of any of the embodiments described herein, the linking group represented by the variable D is —O—, —C(═O)O—, —C(═O)NH— or phenylene. In exemplary embodiments, D is —C(═O)O— or —C(═O)NH—.

For example, the backbone unit may optionally be a vinyl alcohol derivative (e.g., an ester or ether of a vinyl alcohol unit) when Dis-O—; an acrylate or methacrylate derivative (e.g., an ester of an acrylate or methacrylate unit) when D is —C(═O)O—; an acrylamide or methacrylamide unit when D is —C(═O)NH—; and/or a styrene derivative (e.g., a substituted styrene unit) when D is phenylene.

In some embodiments of any of the embodiments described herein, L₁ is a substituted or unsubstituted hydrocarbon from 1 to 10 carbon atoms in length. In some embodiments, the hydrocarbon is unsubstituted. In some embodiments, the hydrocarbon is a linear, unsubstituted hydrocarbon, that is, —(CH₂)ᵢ— wherein i is an integer from 1 to 10, or from 1 to 8, or from 1 to 6, preferably from 1 to 4, or from 1 to 3, or is 1 or 2.

In some embodiments of any of the embodiments described herein, L₁ is a substituted or unsubstituted ethylene group. In some embodiments, L₁ is an unsubstituted ethylene group (—CH₂CH₂—).

In some embodiments of any of the embodiments described herein, B is an oxygen atom. In some such embodiments, L₁ is a hydrocarbon according to any of the respective embodiments described herein (i.e., L₁ is not absent), and Z is a phosphate group attached to L₁.

In some embodiments of any of the embodiments described herein, A is a substituted or unsubstituted hydrocarbon from 1 to 4 carbon atoms in length.

In some embodiments of any of the embodiments described herein, A is an unsubstituted hydrocarbon. In some such embodiments, the unsubstituted hydrocarbon is from 1 to 4 carbon atoms in length. In some embodiments, the hydrocarbon is a linear, unsubstituted hydrocarbon, that is, —(CH₂)ⱼ— wherein j is an integer from 1 to 4.

In some embodiments of any of the embodiments described herein, A is a substituted or unsubstituted ethylene group.

In some embodiments of any of the embodiments described herein, A is an unsubstituted ethylene group (—CH₂CH₂—). In such embodiments, the moiety represented by Formula II (represented by the variable Z) is similar or identical to a phosphoethanolamine or phosphocholine moiety. Phosphoethanolamine and phosphocholine moieties are present in many naturally occurring compounds (e.g., phosphatidylcholines, phosphatidylethanolamines).

In some embodiments of any of the embodiments described herein, A is an ethylene group substituted by a C-carboxy group. In some embodiments, the C-carboxy is attached to the carbon atom adjacent to the nitrogen atom depicted in formula II (rather than the carbon atom attached to the depicted oxygen atom). In such embodiments, the moiety having general formula II (represented by the variable Z) is similar or identical to a phosphoserine moiety. Phosphoserine is present in many naturally occurring compounds (e.g., phosphatidylserines).

Without being bound by any particular theory, it is believed that moieties similar or identical to naturally occurring moieties such as phosphocholine, phosphoethanolamine and/or phosphoserine may be particularly biocompatible.

In some embodiments of any of the embodiments described herein, R₁-R₃ (the substituents of the nitrogen atom depicted in Formula II) are each independently hydrogen or C₁₋₄-alkyl. In some embodiments, R₁-R₃ are each independently hydrogen or methyl. In some embodiments, R₁-R₃ are each methyl.

In some embodiments of any of the embodiments described herein, L₂ is a substituted or unsubstituted hydrocarbon from 1 to 10 carbon atoms in length. In some embodiments, the hydrocarbon is unsubstituted. In some embodiments, the hydrocarbon is a linear, unsubstituted hydrocarbon, that is, —(CH₂)ᵢ— wherein i is an integer from 1 to 10, or from 1 to 8, or from 1 to 6, preferably from 1 to 4, or from 1 to 3, or is 1 or 2.

In some embodiments of any of the embodiments described herein, L₂ is a substituted or unsubstituted ethylene group. In some embodiments, L₂ is an unsubstituted ethylene group (—CH₂CH₂—).

According to the present embodiments, W is a hydrophobic group, such that the polymer features, in addition to the phosphatidyl choline-type moiety Z, a hydrophobic moiety that improves at least the contribution of the co-polymeric compound in reducing friction coefficient of hydrophobic surfaces, as is discussed in further detail hereinbelow.

As used herein, the term “hydrophobic” describes a physical property of a material or a portion of a material (e.g., a chemical group in a compound) which does not form bond(s) with water and tend to associate with non-polar materials. Hydrophobic materials dissolve more readily in oil than in water.

Herein, the phrase “hydrophobic group” describes a chemical moiety that can interact via hydrophobic interactions with a compound (e.g., a surface as defined herein), depending on its chemical nature. It encompasses functional moieties that repel or fail to interact with water and/or other polar substances, and tend to associate with non-polar substances or solvents.

Hydrophobicity is commonly measured by its distribution behavior in a biphasic system: either liquid-liquid (e.g., partition coefficient in 1-octanol/water, as described herein) or solid-liquid (retention on reversed-phase high-performance liquid chromatography (RP-HPLC) or thin-layer chromatography (TLC) system).

Hydrophilic, amphiphilic, and hydrophobic materials can be determined by the partition coefficient thereof. A partition coefficient is the ratio of concentrations of a compound in the two phases of a mixture of two immiscible liquids at equilibrium, typically at room temperature. Normally, one of the solvents chosen is water while the second is hydrophobic such as n-octanol. The logarithm of the ratio of the concentrations of the un-ionized solute in the solvents is called Log P.

Hydrophobic materials are characterized by Log P higher than 1; hydrophilic materials are characterized by Log P lower than 1; and amphiphilic materials are characterized by Log P of about 1 (e.g., 0.8-1.2).

Hydrophobicity can alternatively, or in addition, be determined by a hydrophilic-lipophilic balance (HLB), according to the Davies method, lower than 3.

While Log P and HLB values are typically measured for an entire molecule or substance, the hydrophobicity of substituents or pendant groups in a polymeric or co-polymeric compound can be determined, for example, by the Hansch-Fujita Scale.

The Hansch-Fujita Scale measures the relative hydrophobicity of substituents by using a parameter n, which indicates how the hydrophobicity of a molecule changes when a substituent is introduced or modified.

The π parameter is determined according to the following equation:

π=Log P(substituted compound)−Log P(parent compound)

Positive π values indicate that the substituent increases hydrophobicity (i.e., the substituted compound has a higher log P than the parent compound), and negative values indicate decrease in hydrophobicity.

In some embodiments of any of the embodiments described herein, W is selected capable of providing a co-polymeric compound characterized by Log P of at least 1, or at least 1.5, or at least 2, or at least 2.5, or at least 3, or at least 3.5, or at least 4, or at least 5, or more.

In some embodiments of any of the embodiments described herein, W is an alkyl. In some of any one of the embodiments described herein, W is C₁₋₄-alkyl, or C₁₋₃-alkyl (an alkyl of 1 to 4 or 1 to 3 carbon atoms in length). In some of any one of the embodiments described herein, W is an alkyl of at least 3 carbon atoms, for example, of from 3 to 10, or from 3 to 8, or from 3 to 6, or from 3 to 5, or of 3 or 4 carbon atoms.

In some of any one of the embodiments described herein, W is a branched alkyl. In some of any one of the embodiments described herein, W is a branched alkyl of at least 3 carbon atoms, for example, for example, of from 3 to 10, or from 3 to 8, or from 3 to 6, or from 3 to 5, or of 3 or 4 carbon atoms.

Exemplary branched alkyls include, but are not limited to, isopropyl, isobutyl, isopentyl, amyl, neopentyl, sec-butyl, 2-methylbutyl, 3-methylbutyl, 2-ethylhexyl, isooctyl, 3,3-dimethylbutyl, 3-ethylpentyl, 4-methylhexyl, 3,3-dimethylpentyl, 2,2-dimethylhexyl, and 2-ethyl-3-methylpentyl.

According to some of any of the embodiments described herein, W is a hydrophobic moiety that is characterized by a value of the n parameter, according to the Hansch-Fujita Scale described hereinabove, which is in a range of from 0.5 to 1.2, or from 0.5 to 1.0, including any intermediate values and subranges therebetween. According to some of any of the embodiments described herein, W is a hydrophobic moiety that is characterized by a value of the π parameter, according to the Hansch-Fujita Scale described hereinabove, which is lower than 1.30, or is 1.20 or lower. According to some of any of the embodiments described herein, W is a hydrophobic moiety that is characterized by a value of the π parameter, according to the Hansch-Fujita Scale described hereinabove, which is lower than 1.10, or is lower than 1.00. According to some of any of the embodiments described herein, W is a hydrophobic moiety that is characterized by a value of the π parameter, according to the Hansch-Fujita Scale described hereinabove, within a range of from about 0.5 to about 1.2, or from about 0.5 to about 1.1, or from about 0.5 to about 1., or from about 0.6 to about 1.0, or from about 0.7 to about 1.0, or from about 0.8 to about 1.0 or from about 0.9 to about 1.0. According to some of any of the embodiments described herein, W is a hydrophobic moiety that is characterized by a value of the π parameter, according to the Hansch-Fujita Scale described hereinabove, within a range of from about 0.8 to about 1.20, or from about 0.9 to about 1.20, or from about 0.9 to about 1.10, including any intermediate values and subranges therebetween.

Without being bound by any particular theory, it is assumed that at higher hydrophobicity values of the π parameter hydrophobic interactions with surfaces may adversely affect the capability of the co-polymeric compound to reduce the friction coefficient described hereinbelow.

In some of any one of the embodiments described herein, W is or comprises a branched alkyl of 3 or 4 carbon atoms in length. In some of any one of the embodiments described herein, W is isopropyl. Alternatively, W can be iso-butyl.

In some of any one of the embodiments described herein, E₁ and E₂ each independently represents a terminal group.

The terminal groups can be an intrinsic terminal group, derived from the monomers used to form the co-polymeric compound. For example, E₁ and E₂ can each independently be hydrogen, alkyl, cycloalkyl, hydroxy, carboxy, amine, and the like. In some of any of the embodiments described herein, E₁ is hydrogen or alkyl (e.g., methyl). In some of any of the embodiments described herein E₂ is hydrogen or alkyl (e.g., methyl).

In some of any one of the embodiments described herein, E₁ is or comprises a lipid moiety. In some such embodiments, the lipid moiety is derived from a natural lipid (e.g., phosphatidylcholine, phosphatidylethanolamine, phosphatidylserine, phosphatidylinositol). In some such embodiments, the lipid comprises a moiety derived from a bilayer-forming lipid (e.g., a glycerophospholipid). Additional, non-limiting examples of moieties of a bilayer-forming lipid include sphingolipids (e.g., sphingomyelin), glycolipids (e.g., gangliosides), sterols (e.g., cholesterol), and fatty acids (e.g., oleic acid, palmitic acid).

As used in the art, the term “lipid” describes a class of naturally occurring molecules that are characterized by their hydrophobic or amphiphilic properties, which allow them to form structures such as membranes, vesicles, or droplets in aqueous environments. Suitable examples of lipids include, without limitation, fatty acids, glycerides, phospholipids, sphingolipids, glycolipids, sterols, and other related compounds.

In some embodiments of any of the embodiments described herein, the lipid moiety is a moiety of a lipid which is a fatty acid, a monoglyceride, a diglyceride, a triglyceride, a glycerophospholipid, a sphingolipid, or a sterol. In some embodiments, the lipid is a glycerophospholipid.

In some embodiments of any of the embodiments described herein, the lipid moiety comprises at least one fatty acid moiety (e.g., an acyl group derived from a fatty acid). The fatty acid moiety may be derived from a saturated or unsaturated fatty acid. For example, the lipid moiety may consist of a fatty acid moiety, or be a monoglyceride moiety comprising one fatty acid moiety, a diglyceride moiety comprising two fatty acid moieties, or a triglyceride moiety comprising three fatty acid moieties.

Examples of fatty acid moieties which may optionally be comprised by the lipid moiety include, without limitation, lauroyl, myristoyl, palmitoyl, stearoyl, palmitoleoyl, oleoyl, and linoleoyl.

As used herein and in the art, the term “glycerophospholipids” describes a class of lipids that consist of a glycerol backbone esterified to two fatty acid chains and a phosphate group. The phosphate group is typically further linked to additional polar head groups, such as choline, ethanolamine, serine, or inositol, making glycerophospholipids amphiphilic molecules. Suitable examples of glycerophospholipids include, without limitation, a phosphatidyl ethanolamine, a phosphatidyl serine, a phosphatidyl glycerol and a phosphatidyl inositol.

The variable n may be regarded as representing a number of backbone units (represented by the variable Y₁) which are substituted by the pendant group represented by (-L₁-Z), and the variable m may be regarded as representing a number of backbone units (represented by the variable Y₂) which are substituted by the pendant group represented by (-L₂-W).

In some embodiments of any of the embodiments described herein, n is in a range of from 1 to 10,000, or from 10 to 1,000, or from 50 to 750, or from 100 to 500, or from 100 to 300, including any intermediate values and subranges therebetween.

In some embodiments of any of the embodiments described herein, m is in a range of from 1 to 10,000, or from 10 to 7,500, or from 100 to 5,000, or from 100 to 2,500, or from 1,000 to 2,000, including any intermediate values and subranges therebetween.

A sum of n+m (i.e., the sum of variable n and variable m) may be regarded as representing the total number of backbone units in the polymeric backbone. In some of any one of the embodiments described herein, the sum of n+m is in a range of from 3 to 15,000, or from 3 to 5,000, or from 100 to 5,000, or from 300 to 5,000, or from 500 to 3,000, or from 1,000 to 3,000, or from 1,000 to 2,000, including any intermediate values and subranges therebetween. Higher values are also contemplated.

In some embodiments of any of the embodiments described herein, the number-average molecular weight (Mn) of the co-polymeric compound or the co-polymeric moiety as described herein, as determined by a gel-permeation chromatography (GPC) analysis, is at least 500 or at least 1,000 or at least 5,000 or at least 10,000 or at least 20,000 grams/mol, and can be in a range of, for example, 1,000 to 50,000, or 1,000 to 30,000 or 1,000 to 25,000, or 5,000 to 50,000, or 5,000 to 30,000 or 5,000 to 25,000 or 10,000 to 50,000 or 10,000 to 30,000, or 1,000 to 20,000 or 1,000 to 10,000, or 1,000 to 5,000, grams/mol, including any intermediate values and subranges therebetween.

In some embodiments of any of the embodiments described herein, the molecular weight distribution (Mw/Mn), as determined by a gel-permeation chromatography (GPC) analysis, of the co-polymeric compound or the co-polymeric moiety as described herein is at least 500 or at least 1,000 or at least 5,000 or at least 10,000 or at least 15,000 grams/mol, and can be in a range of, for example, 1,000 to 50,000, or 1,000 to 30,000 or 1,000 to 25,000, or 1,000 to 20,000, or 5,000 to 50,000, or 5,000 to 30,000 or 5,000 to 25,000 or 5,000 to 20,000, or 10,000 to 50,000 or 10,000 to 30,000, or 1,000 to 20,000 or 1,000 to 10,000, or 1,000 to 5,000, grams/mol, including any intermediate values and subranges therebetween.

The ratio of n/m (i.e., the ratio between variable n and variable m) may be regarded as representing a mol ratio between backbone units which are substituted by the pendant group represented by (-L₁-Z) to the backbone units which are substituted by the pendant group represented by (-L₂-W). In some of any one of the embodiments described herein, the ratio of n/m is in a range of from 1:10 to 1:50, or from 1:2 to 1:50, or from 1:3 to 1:10 (n:m), including any intermediate values and subranges therebetween. In exemplary embodiments, the ratio of n/m is 1:7. In some embodiments of any of the embodiments described herein, the percentage of backbone units represented by the variable Y₁ which are substituted by the pendant group represented by (-L₁-Z) (as represented by the formula 100%*n/(n+m), the percentage of variable n from the sum of n+m) is in a range of from 0.01% to 50%, or from 1% to 25%, or from 5% to 25%, or from 7% to 20%, or from 10% to 20%, or from 10% to 15%, including any intermediate values and subranges therebetween.

In some embodiments of any of the embodiments described herein, the percentage of backbone units represented by the variable Y₂ which are substituted by the pendant group represented by (-L₂-W) (as represented by the formula 100%*m/(n+m), the percentage of variable m from the sum of n+m) is in a range of from 50% to 99.99%, or from 60% to 99%, or from 70% to 99%, or from 70% to 95%, or from 80% to 95%, or from 85% to 90%, including any intermediate values and subranges therebetween.

In some embodiments of any of the embodiments described herein, the co-polymeric compound comprises more than two different backbone units. In some such embodiments, the different types of backbone units may differ in whether they comprise the pendant group (-L₁-Z) according to any of the respective embodiments described herein (e.g., when n is at least 2), differ in the type of backbone unit Y₁, and/or differ in the type of pendant group (-L₁-Z).

Herein throughout, the phrases “backbone unit” and “monomeric unit” are used interchangeably to describe the repeating units that are covalently bound to another and form the polymeric backbone chain.

The plurality (indicated by the variable n) of backbone units Y₁ substituted by the pendant group (-L₁-Z) may be the same or different from one another.

In addition, the plurality (indicated by the variable n) of pendant groups (-L₁-Z) attached to a plurality of backbone units Y₁, may be the same or different from one another (e.g., may differ in the identity of any one or more of A, B, R₁, R₂, R₃ and L₁).

For example, in some embodiments of any of the embodiments described herein, the backbone unit Y₁ in each of the Y₁(-L₁-Z) units may optionally be the same or different, while the L₁ and Z moieties are the same among the Y₁(-L₁-Z) units. In some such embodiments, backbone units not substituted by the pendant group (if any such units are present) may optionally be the same as backbone unit Y₁ in each of the Y₁(-L₁-Z) units. Alternatively, backbone units not substituted by the pendant group (if any such units are present) may optionally be different than backbone unit Y₁ in each of the Y₁(-L₁-Z) units (while optionally being the same among all backbone units not substituted by the pendant group).

In some embodiments of any of the embodiments described herein, the L₁ moiety in each of the Y₁(-L₁-Z) units may optionally be the same or different, while the backbone units Y₁ and the Z moieties are the same among the Y₁(-L₁-Z) units. In some such embodiments, backbone units not substituted by the pendant group (if any such units are present) may optionally be the same as backbone unit Y₁ in each of the Y₁(-L₁-Z) units. Alternatively, backbone units not substituted by the pendant group (if any such units are present) may optionally be different than backbone unit Y₁ in each of the Y₁(-L₁-Z) units (while optionally being the same among all backbone units not substituted by the pendant group).

According to some of any of the embodiments described herein, the backbone units represented by the variable Y₁ which are substituted by the pendant group represented by (-L₁-Z), and the backbone units represented by the variable Y₂ which are substituted by the pendant group represented by (-L₂-W), are distributed in the co-polymer randomly, in any order.

In some embodiments of any of the embodiments described herein the Z moiety in each of the Y₁(-L₁-Z) units may optionally be the same or different, while the backbone units Y₁ and the Z moieties are the same among the Y₁(-L₁-Z) units. In some such embodiments, backbone units not substituted by the pendant group (if any such units are present) may optionally be the same as backbone unit Y₁ in each of the Y₁(-L₁-Z) units. Alternatively, backbone units not substituted by the pendant group (if any such units are present) may optionally be different than backbone unit Y₁ in each of the Y₁(-L₁-Z) units (while optionally being the same among all backbone units not substituted by the pendant group).

In some embodiments of any of the embodiments described herein, the co-polymeric compound comprises more than two different monomeric units. In some such embodiments, the different types of monomeric unit may differ in whether they comprise the pendant group (-L₂-W) according to any of the respective embodiments described herein (e.g., when m is at least 2), differ in the type of backbone unit Y₂, and/or differ in the type of pendant group (-L₂-W).

The plurality (indicated by the variable m) of backbone units Y₂ substituted by the pendant group (-L₂-W) may be the same as each other or different from each other.

In addition, the plurality (indicated by the variable m) of pendant groups (-L₂-W) attached to a plurality of backbone units Y₂, may be the same as each other or different from each other (e.g., may differ in the identity of any one or more of W and L₂).

For example, in some embodiments of any of the embodiments described herein the backbone unit Y₂ in each of the Y₂(-L₂-W) units may optionally be the same or different, while the L₂ and W moieties are the same among the Y₂(-L₂-W) units. In some such embodiments, backbone units not substituted by the pendant group (if any such units are present) may optionally be the same as backbone unit Y₂ in each of the Y₂(-L₂-W) units. Alternatively, backbone units not substituted by the pendant group (if any such units are present) may optionally be different than backbone unit Y₂ in each of the Y₂(-L₂-W) units (while optionally being the same among all backbone units not substituted by the pendant group).

In some embodiments of any of the embodiments described herein the L₂ moiety in each of the Y₂(-L₂-W) units may optionally be the same or different, while the backbone units Y₂ and the W moieties are the same among the Y₂(-L₂-W) units. In some such embodiments, backbone units not substituted by the pendant group (if any such units are present) may optionally be the same as backbone unit Y₂ in each of the Y₂(-L₂-W) units. Alternatively, backbone units not substituted by the pendant group (if any such units are present) may optionally be different than backbone unit Y₂ in each of the Y₂(-L₂-W) units (while optionally being the same among all backbone units not substituted by the pendant group).

In some embodiments of any of the embodiments described herein the W moiety in each of the Y₂(-L₂-W) units may optionally be the same or different, while the backbone units Y₂ and the W moieties are the same among the Y₂(-L₂-W) units. In some such embodiments, backbone units not substituted by the pendant group (if any such units are present) may optionally be the same as backbone unit Y₂ in each of the Y₂(-L₂-W) units. Alternatively, backbone units not substituted by the pendant group (if any such units are present) may optionally be different than backbone unit Y₂ in each of the Y₂(-L₂-W) units (while optionally being the same among all backbone units not substituted by the pendant group).

In some embodiments of any of the embodiments described herein, the co-polymeric compound further comprises a targeting moiety. The targeting moiety can be attached to at least a portion of the monomeric units of the co-polymeric compound.

Herein, a “targeting moiety” refers to a moiety which is capable of bringing a compound (e.g., a compound according to some embodiments of the invention) into proximity with a selected substance and/or material (which is referred to herein as a “target”). The target is optionally a cell (e.g., a proliferating cell associated with the proliferative disease or disorder), wherein the proximity is such that the targeting moiety facilitates attachment and/or internalization of the compound into a target cell, and such that the compound may exert a therapeutic effect.

In some embodiments of any of the embodiments described herein, the targeting moiety can be attached (e.g., covalently) to one or more of the Y₁ backbone units, the Y₂ backbone units, the E₁ terminal group, the E₂ terminal group, the L₁ linking group, the L₂ linking group, the Z moiety and/or the W moiety.

A targeting moiety may optionally be comprised by a backbone unit Y₁ according to any of the respective embodiments described herein, a backbone unit Y₂ according to any of the respective embodiments described herein, a linking moiety L₁ according to any of the respective embodiments described herein, a linking moiety L₂ according to any of the respective embodiments described herein, moiety Z according to any of the respective embodiments described herein, moiety W according to any of the respective embodiments described herein, a terminal group E₁ according to any of the respective embodiments described herein, and/or a terminal group E₂ according to any of the respective embodiments described herein, for example, wherein a substituent according to any of the respective embodiments described herein comprises (and optionally consists of) the targeting moiety.

For example, in some embodiments wherein at least a portion of backbone units Y₁ have the formula —CR₄R₅—CR₆D- (as described herein in any of the respective embodiments), any one or more of R₄-R₆ and D comprises a targeting moiety according to any of the respective embodiments described herein (e.g., wherein any one or more of R₄-R₆ and D is a substituted group, comprising a substituent which is a targeting moiety), and optionally any one or more R₄-R₆ and D is a targeting moiety. However, many other structures of monomeric units comprising a substituent which comprises (and optionally consist of) a targeting moiety are also encompassed by embodiments of the invention.

In some of any of the embodiments of the co-polymeric moiety, at least one type of monomeric unit comprises a targeting moiety (according to any of the respective embodiments described herein) and at least one type of monomeric unit does not comprise such a targeting moiety. The distribution of a monomeric unit comprising a targeting moiety may be in accordance with any distribution described herein of a monomeric unit in a co-polymer moiety (e.g., random, alternating, periodic copolymer, and/or block copolymer).

A monomeric unit comprising a targeting moiety according to any of the respective embodiments described herein may optionally be prepared by preparing a monomer comprising a targeting moiety, and using the monomer to prepare a co-polymeric moiety as described herein (e.g., by co-polymerization of monomeric units according to any of the respective embodiments described herein) and/or by modifying a monomeric unit in a co-polymeric moiety subsequently to preparation of a co-polymeric moiety (e.g., by polymerization of monomers according to any of the respective embodiments described herein), using any suitable technique known in the art, including, but not limited to, techniques for conjugation.

In some embodiments of any of the embodiments described herein relating to a targeting moiety, the targeting moiety does not comprise a moiety represented by Formula II (according to any of the respective embodiments described herein). For example, even if a moiety represented by Formula II is capable of forming a bond with a target as described herein, the phrase “targeting moiety”, in some embodiments, is to be understood as relating to a moiety distinct from a moiety represented by variable Z (represented by Formula II).

In some embodiments of any one of the embodiments described herein, the pendant group represented by (-L₁-Z) is selected so as not to form a bond with the target and/or so as not to include a structure and/or property of a targeting moiety as described herein in any one of the respective embodiments. For example, in embodiments wherein a targeting moiety comprising a nucleophilic group (according to any of the respective embodiments described herein)—for example, an amine group—is capable of forming a bond (e.g., covalent bond) with a target, the variable Z (represented by Formula II) is optionally selected such that the depicted amine/ammonium group is a tertiary amine/ammonium (i.e., no more than one of R₁-R₃ is hydrogen) or quaternary ammonium (i.e., none of R₁-R₃ is hydrogen), preferably a quaternary ammonium (e.g., comprising a trimethylamino group, such as in phosphocholine). Tertiary amine groups, and especially quaternary ammonium groups, may be significantly less reactive nucleophilic groups than primary and secondary amine groups.

In some embodiments of any of the embodiments described herein relating to a targeting moiety, the targeting moiety comprises (and optionally consists of) at least one functional group capable of forming a covalent bond or non-covalent bond (preferably a selective non-covalent bond) with a substance and/or material (which is referred to herein as a “target”), e.g., at a surface of the target (e.g., a surface of a cell and/or tissue).

Herein, the phrase “functional group” encompasses chemical groups and moieties of any size and any functionality described herein (for example, any functionality capable of forming a covalent bond or non-covalent bond with a target).

A non-covalent bond according to any of the respective embodiments described herein may optionally be effected by non-covalent interactions such as, without limitation, electrostatic attraction, hydrophobic bonds, hydrogen bonds, and aromatic interactions.

In some embodiments, the targeting moiety comprises a functional group capable of forming a non-covalent bond which is selective for the target, e.g., an affinity (e.g., as determined based on a dissociation constant) of the targeting moiety and/or functional group to the target is greater than an affinity of the of the targeting moiety and/or functional group to most (or all) other compounds capable of forming a non-covalent bond with the targeting moiety.

In some embodiments of any one of the embodiments described herein, the functional group(s) are capable of forming a covalent bond with one or more specific functional groups (e.g., hydroxy, amine, thiohydroxy and/or oxo groups) which are present on the target (e.g., a target according to any of the respective embodiments described herein).

Examples of functional groups (in a targeting moiety) capable of forming a covalent bond with a target (according to any of the respective embodiments described herein) and the type of covalent bonds they are capable of forming, include, without limitation:
nucleophilic groups such as thiohydroxy, amine (e.g., primary or secondary amine) and hydroxy, which may form covalent bonds with, e.g., a nucleophilic leaving group (e.g., any nucleophilic group described herein), Michael acceptor (e.g., any Michael acceptor described herein), acyl halide, isocyanate and/or isothiocyanate (e.g., as described herein) in a target;
nucleophilic leaving groups such as halo, azide (—N₃), sulfate, phosphate, sulfonyl (e.g. mesyl, tosyl), N-hydroxysuccinimide (NHS) (e.g. NHS esters), sulfo-N-hydroxysuccinimide, and anhydride, which may form covalent bonds with, e.g., a nucleophilic group (e.g., as described herein) in a target;
Michael acceptors such as enones (e.g., maleimide, acrylate, methacrylate, acrylamide, methacrylamide), nitro groups and vinyl sulfone, which may form covalent bonds with, e.g., a nucleophilic group (e.g., as described herein) in a target, optionally thiohydroxy;
dihydroxyphenyl groups (according to any of the respective embodiments described herein), which may form covalent bonds with, e.g., a nucleophilic group (e.g., as described herein) and/or a substituted or unsubstituted phenyl group (e.g., another dihydroxyphenyl group) in a target, as described herein;
an acyl halide (—C(═O)-halogen), isocyanate (—NCO) and isothiocyanate (—N═C═S) group, which may form covalent bonds with, e.g., a nucleophilic group (e.g., as described herein) in a target;
a carboxylate (—C(═O)OH) group, which may form a covalent bond with, e.g., a hydroxyl group in a target to form an ester bond and/or an amine group (e.g., primary amine) in a target to form an amide bond (optionally by reaction with a coupling reagent such as a carbodiimide); and/or a carboxylate group is in a target and may form an amide or ester bond with an amine or hydroxyl group, respectively, in the targeting moiety;
an oxo group (optionally in an aldehyde group (—C(═O)H)), which may form a covalent imine bond with an amine group (e.g., a primary amine) in a target; and/or an oxo group (optionally in an aldehyde group) is in a target and may form a covalent imine bond with an amine groups in the targeting moiety; and/or
thiohydroxy groups, which may form covalent disulfide (—S—S—) bonds with a thiohydroxy group in a target.

Modification of a monomer (e.g., prior to polymerization or co-polymerization) or a monomeric unit of a co-polymeric moiety (e.g., subsequent to polymerization or co-polymerization) to comprise any of the functional groups described herein may optionally be performed using any suitable technique for conjugation known in the art. The skilled person will be readily capable of selecting a suitable technique for any given molecule to be modified.

Herein, the term “dihydroxyphenyl” refers to an aryl group (as defined herein) which is a phenyl substituted by two hydroxyl groups at any positions thereof. The phenyl may optionally be substituted by additional substituents (which may optionally comprise additional hydroxyl groups), to thereby form a substituted dihydroxyphenyl group; or alternatively, the phenyl comprises no substituents other than the two hydroxyl groups, such that the dihydroxyphenyl group is an unsubstituted dihydroxyphenyl group.

In some embodiments of any one of the embodiments described herein, the dihydroxyphenyl group is an ortho-dihydroxyphenyl (wherein the hydroxyl groups are attached to the phenyl at adjacent positions) or a para-dihydroxyphenyl (wherein the hydroxyl groups are attached to opposite sides of the phenyl ring), each being a substituted or unsubstituted dihydroxyphenyl. In some such embodiments, the ortho-dihydroxyphenyl or para-dihydroxyphenyl is an unsubstituted dihydroxyphenyl.

A dihydroxyphenyl group according to any of the respective embodiments described herein may optionally bond covalently and/or non-covalently to a target according to any one or more attachment mechanism described for dihydroxyphenyl (catechol) groups in Lee et al. [PNAS 2006, 103:12999-13003], Brodie et al. [Biomedical Materials 2011, 6, 015014] and/or International Patent Application PCT/IL2015/050606, the contents of each of which are incorporated in their entirety, and especially contents regarding bonds formed by dihydroxyphenyl (catechol) groups to surfaces.

In some embodiments of any one of the embodiments described herein, the functional group capable of forming a bond to a target is a functional group capable of forming a covalent bond with an amine group, optionally a primary amine group. In some such embodiments, the target comprises on or more amino acids or amino acid residues, for example, a peptide or polypeptide of any length (e.g., at least two amino acid residues, for example, proteins), and the amine groups may optionally be lysine side chain amine groups and/or N-terminal amine groups. In some embodiments, the target comprises an extracellular matrix protein, for example, collagen. In some embodiments, the target comprises cartilage (e.g., articular cartilage).

In some embodiments of any one of the embodiments described herein, the targeting moiety comprises (and optionally consists of) at least one functional group capable of forming a non-covalent bond with the target (e.g., as described herein in any one of the respective embodiments).

In some embodiments of any one of the embodiments described herein, a functional group capable of forming a non-covalent bond with the target comprises (and optionally consists of) a polysaccharide and/or polypeptide (e.g., a protein and/or fragment thereof), wherein the target optionally comprises a ligand of the polysaccharide and/or polypeptide; and/or the target comprises a polysaccharide and/or polypeptide (e.g., a protein and/or fragment thereof) and the functional group capable of forming a non-covalent bond with the target is a ligand of the polysaccharide and/or polypeptide.

Examples of suitable polysaccharides and/or polypeptides, and ligands thereof, include, without limitation: avidin or streptavidin as a polypeptide described herein, and biotin as a ligand thereof; a polysaccharide-binding polypeptide as a polypeptide described therein, and a complementary polysaccharide as a ligand thereof (or a complementary polysaccharide-binding polypeptide as a ligand of a polysaccharide described herein); a collagen-binding polypeptide as a polypeptide described therein, and a complementary collagen as a ligand thereof (or a collagen as a polypeptide described herein and a complementary collagen-binding polypeptide as a ligand thereof); a cell receptor expressed by a cell, and a ligand selectively bound by the receptor; an antibody towards any antigen (e.g., wherein the target described herein optionally comprises the antigen) or a fragment of such an antibody as a polypeptide described herein, and the respective antigen as a ligand thereof; and an antibody mimetic towards any antigen (e.g., wherein the target described herein optionally comprises the antigen).

Examples of cell receptors expressed by a cell include, without limitation, receptors characteristic of a particular type of cell and/or tissue, and receptors overexpressed by a cancer cell. The cell receptor or the cell is optionally a target described herein, and the targeting moiety optionally comprises any ligand of the receptor. Examples of such ligands include, without limitation, transferrin, a ligand of transferrin receptor which may optionally target transferrin receptor overexpressed by some cancer cells; keratinocyte growth factor (KGF or FGF7) which is specific for cells of epithelial origin, and may optionally target KGF receptor such as that overexpressed by an endometrial carcinoma or pancreatic carcinoma [Visco et al., Int J Oncol 1999, 15:431-435; Siegfried et al., Cancer 1997, 79:1166-1171]; and epidermal growth factor (EGF) which may optionally target an EGF receptor, optionally an erbB, such as that overexpressed by gliomas and endometrial carcinomas [Normanno et al., Curr Drug Targets 2005, 6:243-257]).

As used herein, the term “antibody” encompasses any type of immunoglobin.

As used herein, the phrase “antibody mimetic” encompasses any type of molecule, optionally a polypeptide, referred as such in the art capable of selectively binding an antigen (e.g., non-covalently). Non-limiting examples of antibody mimetics include affibodies, affilins, affimers, affitins, alphabodies, anticalins, avimers, DARPins, Fynomers, Kunitz domain peptides, and monobodies, e.g., as described in Nygren [FEBS J 2008, 275:2668-2676], Ebersbach et al. [J Mol Biol 2007, 372:172-185], Johnson et al. [Anal Chem 2012, 84:6553-6560], Krehenbrink et al. [J Mol Biol 2008, 383:1058-1068], Desmet et al. [Nature Comm 2014, 5:5237], Skerra [FEBS J 2008, 275:2677-2683], Silverman et al. [Nature Biotechnol 2005, 23:1556-1561], Stumpp et al. [Drug Discov Today 2008, 13:695-701], Grabulovski et al. [J Biol Chem 2007, 282:3196-3204], Nixon & Wood [Curr Opin Drug Discov Devel 2006, 9:261-268], Koide & Koide [Methods Mol Biol 2007, 325:95-109], and Gebauer & Skerra [Curr Opin Chem Biol 2009, 13:245-255], the contents of each of which are incorporated in their entirety, and especially contents regarding particular types of antibody mimetics.

As used herein, the phrase “polysaccharide-binding polypeptide” encompasses any polypeptide or oligopeptide (peptide chains of at least 2, and preferably at least 4 amino acid residues in length) capable of selectively binding (e.g., non-covalently) to a polysaccharide. A wide variety of polysaccharide-binding polypeptides and their binding specificities will be known to the skilled person, and include short peptide sequences (e.g., from 4 to 50, optionally 4 to 20 amino acid residues in length), and longer polypeptides such as proteins or fragments (e.g., carbohydrate-binding modules and/or domains) thereof. In addition, the phrase “polysaccharide-binding polypeptide” encompasses antibodies capable of specifically binding to a polysaccharide. Such antibodies will be available to the skilled person and/or the skilled person will know how to prepare such antibodies, using immunological techniques known in the art.

Examples of polysaccharide-binding polypeptides which may be used in some of any one of the embodiments of the invention include, without limitation, carbohydrate-binding modules (CBMs); and hyaluronic acid-binding peptides, polypeptides and/or modules (e.g., having a sequence as described in any of International Patent Application publication WO 2013/110056; International Patent Application publication WO 2014/071132; Barta et al. [Biochem J 1993, 292:947-949], Kohda et al. [Cell 1996, 86:767-775], Brisset & Perkins [FEBS Lett 1996, 388:211-216], Peach et al. [J Cell Biol 1993, 122:257-264], Singh et al. [Nature Materials 2014, 13:988-995], and Zaleski et al. [Antimicrob Agents Chemother 2006, 50:3856-3860], the contents of each of which are incorporated in their entirety, and especially contents regarding particular polysaccharide-binding polypeptides), for example, GAHWQFNALTVR (a hyaluronic acid-binding peptide sequence).

Examples of CBMs which may be used in some of any one of the embodiments of the invention, include, without limitation, CBMs belonging to the families CBM3, CBM4, CBM9, CBM10, CBM17 and/or CBM28 (which may optionally be used to bind cellulose, e.g., in a cellulose-containing target); CBM5, CBM12, CBM14, CBM18, CBM19 and/or CBM33 (which may optionally be used to bind chitin and/or other polysaccharides comprising N-acetylglucosamine, e.g., in a chitin-containing target); CBM15 (which may optionally be used to bind hemicellulose, e.g., in a hemicellulose-containing target); and/or CBM20, CBM21 and/or CBM48 (which may optionally be used to bind starch and/or glycogen, e.g., in a starch-containing and/or glycogen-containing target).

As used herein, the phrase “collagen-binding polypeptide” encompasses any polypeptide or oligopeptide (peptide chains of at least 2, and preferably at least 4 amino acid residues in length) capable of selectively binding (e.g., non-covalently) to a collagen (e.g., one type of collagen, some types of collagen, all types of collagen), including glycosylated polypeptides and oligopeptides such as peptidoglycans and proteoglycans. A wide variety of collagen-binding polypeptides and their binding specificities will be known to the skilled person, and include short peptide sequences (e.g., from 4 to 50, optionally 4 to 20 amino acid residues in length), and longer polypeptides such as proteins or fragments (e.g., collagen-binding domains) thereof. In addition, the phrase “collagen-binding polypeptide” encompasses antibodies capable of specifically binding to a collagen. Such antibodies will be available to the skilled person and/or the skilled person will know how to prepare such antibodies, using immunological techniques known in the art.

Examples of collagen-binding polypeptides which may be used in embodiments of the invention include, without limitation, collagen-binding proteins (e.g., decorin), fragments thereof and/or other polypeptides as described in U.S. Pat. No. 8,440,618, Abd-Elgaliel & Tung [Biopolymers 2013, 100:167-173], Paderi et al. [Tissue Eng Part A 2009, 15:2991-2999], Rothenfluh et al. [Nat Mater 2008, 7:248-254] and Helms et al. [J Am Chem Soc 2009, 131:11683-11685] (the contents of each of which are incorporated in their entirety, and especially contents regarding particular collagen-binding polypeptides), for example, the sequence WYRGRL.

In alternative embodiments, the polymeric moiety does not comprise a targeting moiety described herein according to any of the respective embodiments.

An exemplary general synthetic pathway for co-polymeric compounds represented by Formula I is described herein:

A monomeric unit comprising phospholipid headgroups (e.g., highly hydrated phosphatidylcholine (PC), 2-methacryloyloxyethylphosphorylcholine (MPC)), an N-substituted acrylamide monomeric unit (e.g., NIPAM), and an oxidizing agent (e.g., APS) are dissolved in a solvent (e.g., water). The solution is deoxygenated (e.g., by nitrogen for 30 minutes). After injecting with a polymerization accelerator (e.g., TMEDA), polymerization is carried out (e.g., at room temperature for 16 hours) under a deoxygenated atmosphere (e.g., nitrogen atmosphere). The reaction mixture is dialyzed (e.g., against pure water for 48 hours), and dehydrated (e.g., using a freeze-drying technique), to thereby obtain the random co-polymeric compound. The ratio between the monomers in the random co-polymeric compound is confirmed by comparing the respective signal from each monomer (e.g., the characteristic peak from each monomer) in a ¹H NMR spectrum of the co-polymeric compound.

Alternatively, the random co-polymeric compound represented by Formula I may be synthesized in accordance with other known methods for co-polymerization.

### Compositions and Composite Materials:

According to an aspect of some embodiments of the invention, there is provided a composition comprising a hydrogel or a composite material containing a hydrogel. According to some of any of the embodiments described herein, the hydrogel has incorporated therein the co-polymeric compound as described herein in any of the embodiments.

Herein and in the art, the term “hydrogel” describes a three-dimensional fibrous network containing at least 20%, typically at least 50%, or at least 80%, and up to about 99.99% (by mass) water. A hydrogel can be regarded as a material, which is mostly water, yet behaves like a solid or semi-solid due to a three-dimensional crosslinked solid-like network, made of natural and/or synthetic polymeric chains, within the liquid dispersing medium. According to some embodiments of the present invention, a hydrogel may contain polymeric chains of various lengths and chemical compositions, depending on the precursors used for preparing it. The polymeric chains can be made of monomers, oligomers, block-polymeric units, which are inter-connected (crosslinked) by chemical bonds (covalent, hydrogen and ionic/complex/metallic bonds, typically covalent bonds). The network-forming material comprises either small aggregating molecules, particles, or polymers that form extended elongated structures with interconnections (the crosslinks) between the segments. The crosslinks can be in the form of covalent bonds, coordinative, electrostatic, hydrophobic, or dipole-dipole interactions or chain entanglements between the network segments.

Herein and in the art, the phrase “crosslinking degree” describes the percentage of crosslinks or interconnections between polymeric chains or other network segments within the hydrogel (i.e., the hydrogel-forming agents). The degree of crosslinking affects the mechanical properties of the hydrogel, for example, a higher degree of crosslinking typically results in a stiffer hydrogel, while a lower degree of crosslinking typically allows for increased flexibility and water absorption.

According to some of any of the embodiments described herein, the co-polymeric compound as described herein in any of the respective embodiments is incorporated in the hydrogel in a form of liposomes as described herein in any of the respective embodiments.

According to some of any of the embodiments described herein, an amount of the co-polymeric compound is in a range of from 0.1% to 50%, or from 0.1% to 25%, or from 0.1% to 10%, or from 0.1% to 5%, by weight, of the total weight of the composition or the composite material, or the hydrogel, including any intermediate values and subranges therebetween.

According to some of any of the embodiments described herein, a degree of crosslinking of the hydrogel is in a range of from 0.1 to 15, or from 1 to 15, or from 1 to 10, or from 1 to 5, or is 2, %, including any intermediate values and subranges therebetween.

According to some of any of the embodiments described herein, the hydrogel is formed of at least one hydrogel-forming agent.

As used herein, the phrase “hydrogel-forming agent” describes an agent that is capable of forming a hydrogel as defined herein. Non-limiting examples of hydrogel-forming agents include hydroxyethyl methacrylate (HEMA), hydroxyethyl acrylate (HEA), acrylamide (AAm), methacrylamide (MAAm), acrylic acid (AAc), methacrylic acid (MAAc), hexyl methacrylate, N-isopropylacrylamide (NIPAM)), N-isopropylmethacrylamide, polylactic acid, polyamide, polyethylene-terephthalate (PET), polyvinyl alcohol, polyurethane, polycaprolactone, polyethylene-glycol (PEG), polyethylene-glycol methacrylate (PEGMA), polyethyleneoxide dimethacrylate (PEOdMA), N,N-dimethacrylamide (nnDMAA), hyaluronic acid (HA), HA methacrylate, peptides, saccharides, gelatin, gelatin methacrylate, chitosan, chitosan methacrylate, glycol chitosan, glycol chitosan methacrylate, alginate, alginate methacrylate, cellulose, siloxanes, polysiloxanes, and any oligomer, polymer and/or copolymer thereof, in any combination thereof.

In some of any of the embodiments described herein, the hydrogel comprises hydroxyethyl methacrylate (HEMA) and/or poly hydroxyethyl methacrylate (pHEMA) as hydrogel-forming agents.

According to some of any of the embodiments described herein, the composition or composite material as described in any one of the embodiments herein, and any combination thereof, is in a dry form. By “dry form”, it is meant that the composition or composite material comprises a minimal amount of moisture and/or solvents, for example, up to 10%, or up to 5%, or up to 4%, or up to 3%, or up to 2%, or up to 1%, water and/or other solvents by weight of the total weight of the composition or composite material. Non-limiting examples for dry forms include solids, powders, and granulates. It is to be appreciated that in a dry form, the material is free from significant water content, making it easier to store and suitable for reconstitution or use in further formulations when water and/or other solvents are added.

In some of any of the embodiments described herein, the dry form of the composition or the composite material can be rehydrated.

According to some of any of the embodiments described herein, the composition or composite material is characterized by a storage modulus G′ (at 1 Hz) of at least 10 MPa, and up to 1000 MPa, or of from 10 MPa to 500 MPa, or from 10 MPa to 100 MPa, or from 10 MPa to 50 MPa, or from 100 MPa to 1000 MPa, or from 100 MPa to 500 MPa, or from 500 MPa to 1000 MPa, including any intermediate values and subranges therebetween.

According to some of any of the embodiments described herein, the composition or composite material is characterized by a loss modulus G″ (at 1 Hz) of at least 1,000 Pa, and up to 50,000 Pa, or from 1,000 Pa to 30,000 Pa, or from 1,000 Pa to 10,000 Pa, or from 1,000 Pa to 5,000 Pa, or from 5,000 Pa to 30,000 Pa, or from 5,000 Pa to 50,000 Pa, or from 5,000 Pa to 10,000 Pa, or from 10,000 Pa to 30,000 Pa, or from 10,000 Pa to 50,000 Pa, or from 30,000 Pa to 50,000 Pa, including any intermediate values and subranges therebetween.

According to some of any of the embodiments described herein, the composition or composite material is characterized by a sliding friction coefficient μ lower than 0.05, or lower than 0.01, or lower than 0.001, or even lower, (e.g., lower than 0.0001), including any intermediate values and subranges therebetween. According to some of any of the embodiments described herein, the composition or composite material is characterized by such a sliding friction coefficient μ on a polyethylene (hydrophobic) sphere. In some of any of the embodiments described herein, the composition or composite material is characterized by a sliding friction coefficient μ in a range of from 0.0001 to 0.01, or from 0.005 to 0.01, or from 0.001 to 0.01, or from 0.05 to 0.01, or from 0.0001 to 0.05, or from 0.005 to 0.05, or from 0.001 to 0.05, or from 0.005 to 0.001, or from 0.0001 to 0.005, including any intermediate values and subranges therebetween. According to some of any of the embodiments described herein, the composition or composite material is characterized by such a sliding friction coefficient μ on a polyethylene (hydrophobic) sphere.

According to some of any of the embodiments described herein, the composition or composite material is characterized by a storage modulus G′ (at 1 Hz) of at least 10 MPa, and by a loss modulus G″ (at 1 Hz) of at least 1,000 Pa.

According to some of any of the embodiments described herein, the composition or composite material is characterized by a storage modulus G′ (at 1 Hz) of from 10 MPa to 1000 MPa, or of from 10 MPa to 500 MPa, or from 10 MPa to 100 MPa, or from 10 MPa to 50 MPa, or from 100 MPa to 1000 MPa, or from 100 MPa to 500 MPa, or from 500 MPa to 1000 MPa, and by a loss modulus G″ (at 1 Hz) of from 1,000 Pa to 50,000 Pa, or from 1,000 Pa to 30,000 Pa, or from 1,000 Pa to 10,000 Pa, or from 1,000 Pa to 5,000 Pa, or from 5,000 Pa to 30,000 Pa, or from 5,000 Pa to 50,000 Pa, or from 5,000 Pa to 10,000 Pa, or from 10,000 Pa to 30,000 Pa, or from 10,000 Pa to 50,000 Pa, or from 30,000 Pa to 50,000 Pa.

According to some of any of the embodiments described herein, the composition or composite material is characterized by a storage modulus G′ (at 1 Hz) of at least 10 MPa, and by a sliding friction coefficient μ lower than 0.05 on a polyethylene (hydrophobic) sphere.

According to some of any of the embodiments described herein, the composition or composite material is characterized by a storage modulus G′ (at 1 Hz) of from 10 MPa to 1000 MPa, or of from 10 MPa to 500 MPa, or from 10 MPa to 100 MPa, or from 10 MPa to 50 MPa, or from 100 MPa to 1000 MPa, or from 100 MPa to 500 MPa, or from 500 MPa to 1000 MPa, and by a sliding friction coefficient μ of from 0.0001 to 0.01, or from 0.005 to 0.01, or from 0.001 to 0.01, or from 0.05 to 0.01, or from 0.0001 to 0.05, or from 0.005 to 0.05, or from 0.001 to 0.05, or from 0.005 to 0.001, or from 0.0001 to 0.005, on a polyethylene (hydrophobic) sphere, including any intermediate values and subranges therebetween.

According to some of any of the embodiments described herein, the composition or composite material is characterized by a loss modulus G″ (at 1 Hz) of at least 1,000 Pa, and by a sliding friction coefficient μ lower than 0.05 on a polyethylene (hydrophobic) sphere.

According to some of any of the embodiments described herein, the composition or composite material is characterized by a loss modulus G″ (at 1 Hz) of from 1,000 Pa to 50,000 Pa, or from 1,000 Pa to 30,000 Pa, or from 1,000 Pa to 10,000 Pa, or from 1,000 Pa to 5,000 Pa, or from 5,000 Pa to 30,000 Pa, or from 5,000 Pa to 50,000 Pa, or from 5,000 Pa to 10,000 Pa, or from 10,000 Pa to 30,000 Pa, or from 10,000 Pa to 50,000 Pa, or from 30,000 Pa to 50,000 Pa, and by a sliding friction coefficient μ of from 0.0001 to 0.01, or from 0.005 to 0.01, or from 0.001 to 0.01, or from 0.05 to 0.01, or from 0.0001 to 0.05, or from 0.005 to 0.05, or from 0.001 to 0.05, or from 0.005 to 0.001, or from 0.0001 to 0.005, on a polyethylene (hydrophobic) sphere, including any intermediate values and subranges therebetween.

According to some of any of the embodiments described herein, the composition or composite material is characterized by a storage modulus G′ (at 1 Hz) of at least 10 MPa, by a loss modulus G″ (at 1 Hz) of at least 1,000 Pa, and by a sliding friction coefficient μ lower than 0.05 on a polyethylene (hydrophobic) sphere.

According to some of any of the embodiments described herein, the composition or composite material is characterized by a storage modulus G′ (at 1 Hz) of from 10 MPa to 1000 MPa, or of from 10 MPa to 500 MPa, or from 10 MPa to 100 MPa, or from 10 MPa to 50 MPa, or from 100 MPa to 1000 MPa, or from 100 MPa to 500 MPa, or from 500 MPa to 1000 MPa, and by a loss modulus G″ (at 1 Hz) of from 1,000 Pa to 50,000 Pa, or from 1,000 Pa to 30,000 Pa, or from 1,000 Pa to 10,000 Pa, or from 1,000 Pa to 5,000 Pa, or from 5,000 Pa to 30,000 Pa, or from 5,000 Pa to 50,000 Pa, or from 5,000 Pa to 10,000 Pa, or from 10,000 Pa to 30,000 Pa, or from 10,000 Pa to 50,000 Pa, or from 30,000 Pa to 50,000 Pa, and by a sliding friction coefficient μ of from 0.0001 to 0.01, or from 0.005 to 0.01, or from 0.001 to 0.01, or from 0.05 to 0.01, or from 0.0001 to 0.05, or from 0.005 to 0.05, or from 0.001 to 0.05, or from 0.005 to 0.001, or from 0.0001 to 0.005, on a polyethylene (hydrophobic) sphere, including any intermediate values and subranges therebetween.

According to some of any of the embodiments described herein, the composition or composite material further comprises at least one additional agent, such as, but not limited to, a polymeric or co-polymeric compound, a hydrogel-forming polymer, a lipid, a sterol (e.g., cholesterol), a liposome-stabilizing agent, a labeling agent, a bioactive agent and/or a therapeutically active agent, as described in further detail herein.

As used herein, the term “hydrogel-forming polymer” refers to a polymeric compound, as described herein, which also serves as a hydrogel-forming agent, as described herein.

As used herein, the term “sterol” encompasses all sterols derived from any source and includes synthetic sterols, animal-derived sterols, and plant-derived sterols (hereinafter termed “phytosterols”), as well as the saturated forms of sterols thereof (i.e., stanols). Thus, the term “sterols” as used herein encompasses both sterols and stanols. Sterols are steroids with a hydroxyl group at C₃ (steroid alcohol) and most of the skeleton of cholestane (IUPAC Steroid Nomenclature, 1987). Additional carbon atoms may be present in the side chain, usually in the C₁₇ position. In nature, sterols are found as C₂₆-C₃₀ steroid alcohols. The cylcopentanoperhydrophenanthrene ring structure is common to all sterols, while the side chains may vary in structure. In nature, sterols may be found as conjugates (e.g., glycoconjugates, lipo-conjugates, etc.). Thus, the term “sterol,” as used herein, is further intended to encompass conjugated sterols, including, but not limited to, phytosteryl fatty-acid esters and phytostanyl fatty-acid esters. An exemplary sterol is cholesterol.

According to some of any of the embodiments, the liposome-stabilizing agent is a polymer. According to some embodiments, the polymer can be poly(2-hydroxyethyl methacrylate) (pHEMA), alginate and/or hyaluronic acid (HA). In some of any of the embodiments, the concentration of the polymer in the liposome preparation solution ranges from 0.1 mg polymer per 1 ml of liposome suspension solution to 5 mg/ml, including any intermediate values and subranges therebetween.

As used herein, the phrase “biologically active agent”, or “bioactive agent”, describes a chemical substance, which exhibits a biological or physiological activity in an organism. In some embodiments, a bioactive agent is a therapeutically active agent, such as described herein.

According to an aspect of some embodiments of the present invention, there is provided a process of preparing the composition or composite material as described herein, in any one of the embodiments thereof, and any combination thereof. According to some embodiments, the process is effected by contacting an aqueous solution or suspension comprising a hydrogel-forming agent as described in any of the respective embodiments herein, and a crosslinking agent, with the co-polymeric compound as described in any of the respective embodiments herein, thereby obtaining the composition or composite material.

According to some of any of the embodiments described herein, the aqueous solution or suspension further comprises an oxidizing agent. In exemplary embodiments, the oxidizing agent is or comprises ammonium persulfate (APS). Any other suitable oxidizing agents are contemplated. According to some of any of the embodiments described herein, the aqueous solution or suspension further comprises a catalyst. In exemplary embodiments, the catalyst is or comprises tetramethylethylenediamine (TMEDA). Without being bound to any particular theory, it is assumed that TMEDA acts as co-catalyst by accelerating radical formation of the oxidizing agent. Any other suitable catalysts are contemplated.

Exemplary crosslinking agents that are usable in the context of these embodiments include, but are not limited to, poly(ethylene glycol) n dimethacrylate (EGDMA), N,N′-methylenebis(acrylamide) (MBAm), N,N′-methylenebis(2-methylacrylamide), methylene diacrylate, methylene bis(2-methylacrylate), diethylene glycol diacrylate, hexamethylene diacrylate, oxybis(methylene)bis(2-methylacrylate), oxybis(ethane-2,1-diyl) bis(2-methylacrylate). Additional examples of crosslinking agents include, but are not limited to, glutaraldehyde, genipin, bisphenol A diglycidyl ether (DGEBA), toluene diisocyanate (TDI), polyethylene glycol diisocyanate (PEGDI), methylenebis(phenyl isocyanate) (MDI), sulfur dichloride, epichlorohydrin, tetraethyl orthosilicate (TEOS), and tris(hydroxymethyl) aminomethane (Tris). Any other crosslinking agent is contemplated.

According to some of any of the embodiments described herein, the process further comprises dehydrating the composition or composite material, to thereby obtain the composition or composite material in a dry form as described herein. In some such embodiments, the process may further comprise, subsequent to the dehydrating, rehydrating the composition or composite material, to thereby obtain a rehydrated form of the composition or the composite material as described herein. As demonstrated in the Examples section that follows, dehydration and rehydration of the composition or composite material according to some of the present embodiments can be effected repeatedly as desired, substantially without affecting the friction coefficient of the composition or composite material.

In some such embodiments, the friction coefficient of the composition or the composite material is substantially the same as the friction coefficient of the rehydrated form of the composition or the composite material, for example, the friction coefficient of the composition or the composite material is up to 30% lower or higher than the friction coefficient of the rehydrated form of the composition or the composite material. In some such embodiments, the friction coefficient of the composition or the composite material is up to 20% lower or higher than the friction coefficient of the rehydrated form of the composition or the composite material. In some such embodiments, the friction coefficient of the composition or the composite material is up to 10% lower or higher than the friction coefficient of the rehydrated form of the composition or the composite material. In some such embodiments, the friction coefficient of the composition or the composite material is from 1% to 30%, or from 1% to 20%, or from 1% to 10%, or from 2% to 30%, or from 2% to 20%, or from 2% to 10%, or from 3% to 30%, or from 3% to 20%, or from 3% to 10%, or from 4% to 30%, or from 4% to 20%, or from 4% to 10%, or from 5% to 30%, or from 5% to 20%, or from 5% to 10%, lower or higher than the friction coefficient of the rehydrated form of the composition or the composite material, including any intermediate values and subranges therebetween.

In some of any of the embodiments described herein, the method of incorporating a co-polymeric compound as described herein in any of the respective embodiments and any combination thereof in a hydrogel may be effected such that the tensile strength of the resulting composition or composite material is augmented thereby rendering the composition or composite material useful in applications in which the neat hydrogel was not.

Since a hydrogel can be characterized by its degree of crosslinking, which is also correlated to its tensile strength and its ability to disperse and stabilize the co-polymeric compound, the method of lowering the friction coefficient of a hydrogel is effected, according to some of the embodiments presented herein, for hydrogels which are characterized by a molar percentage of the crosslinking agent that ranges from 0.00001% to 50% per hydrogel-forming agent total molar content. In some embodiments, a mol % of the crosslinking agent as defined herein to the hydrogel-forming agent as defined herein is in a range of from 0.1% to 50%, from 0.5% to 25%, from 1% to 10% or from 1% to 5%, including any intermediate values and subranges therebetween.

According to an aspect of some embodiments of the invention, there is provided a composition or composite material prepared by the process as described herein in any of the respective embodiments.

A hydrogel, as defined herein, may contain macromolecular polymeric and/or fibrous elements which are not chemically connected to the main crosslinked network but are rather mechanically intertwined therewith and/or immersed therein. Such macromolecular fibrous elements can be woven (as in, for example, a mesh structure), or non-woven, and can, in some embodiments, serve as reinforcing materials of the hydrogel's fibrous network. Non-limiting examples of such macromolecules include polycaprolactone, gelatin, gelatin methacrylate, alginate, alginate methacrylate, chitosan, chitosan methacrylate, glycol chitosan, glycol chitosan methacrylate, hyaluronic acid (HA), HA methacrylate, and other non-crosslinked natural or synthetic polymeric chains and the likes. According to some of any of the embodiment of the present invention, the amount of such non-crosslinked additives is small and typically does not exceed 100 mg in 1 ml of the hydrogel-forming precursor solution.

In the context of embodiments of the present invention, when a hydrogel is used in combination with such macromolecular structures, it is referred to interchangeably as “a composite material comprising a hydrogel”, “a composite structure comprising a hydrogel”, “hydrogel-containing composite material or structure”, “hydrogel-containing composite” or simply as “a hydrogel” or as a “composite”. Thus, the term “hydrogel” is meant to encompass hydrogel/fiber-network composites wherein a network of intertwined and/or woven fibers is engulfed in a mass of a hydrogel. In general, a composite material comprising a hydrogel may further comprise a woven mesh of fibers, non-woven fibers, a plurality of rods, a net etc. Exemplary hydrogel/fiber-network composites are described, for example, in Moutos et al. Nat. Mater., 2007, 6 (2), p. 162-7.

Exemplary materials which the composition or composite material comprise include, but are not limited to, a woven mesh of fibers, non-woven fibers, a plurality of rods, and a net.

### Lubrication:

According to an aspect of some embodiments of the invention, there is provided a method of reducing a friction coefficient of a hydrogel or of a composition or composite material comprising a hydrogel as described herein in any of the respective embodiments. According to some of any of the embodiments described herein, lowering a friction coefficient of a hydrogel or of a composition or composite material containing a hydrogel is effected by forming the hydrogel as described herein in any of the respective embodiments and in any combination thereof, in the presence of the co-polymeric compound as described herein in any of the respective embodiments and in any combination thereof. In some such embodiments, forming the hydrogel in the presence of the co-polymeric compound is such that the co-polymeric compound is dispersed throughout the bulk of the hydrogel, thereby lowering the friction coefficient of the resulting composition compared to the friction coefficient of the neat hydrogel (not having co-polymeric compound dispersed therein).

It should be noted that the capacity to lower the friction coefficient of any given hydrogel is based on the premise that the neat hydrogel is such that the notion of having a friction coefficient is relevant thereto, namely that the neat hydrogel is hard enough to allow its friction coefficient to be measured or assessed by conventional means.

According to some embodiments of the present invention, the method is applied to hydrogels or composite materials containing a hydrogel as described herein in any one of the respective embodiments.

As is further demonstrated in the Examples section below, the ability to lower the friction coefficient of a hydrogel depends to some extent on the amount of co-polymeric compound dispersed therein.

According to some embodiments of the present invention, the method is effected by forming the hydrogel in the presence of the co-polymeric compound such that the amount of co-polymeric compound dispersed in the hydrogel is at least 0.00001%, or at least 0.0001%, or at least 0.001%, or at least 0.01%, or at least 0.1%, or at least 1%, as defined herein, including any intermediate values and subranges therebetween.

As demonstrated in the Examples section below, incorporation of a co-polymeric compound as described herein in any of the respective embodiments into the bulk of hydrogels affords hydrogels which are characterized by a dynamic friction coefficient in aqueous media that ranges from 0.0001 to 0.2 under a pressure of at least 1.5 N. These values are not only low in comparison to neat hydrogels, but are notably low in absolute terms even in comparison to lubricated systems in aqueous media.

Unless stated otherwise, the friction coefficient values presented in the Examples section that follows below and referred to hereinthroughout, refer to dynamic friction coefficient, however, the general propensity to lower the friction coefficient of hydrogels is evident in both static and dynamic friction coefficients.

According to some embodiments of the present invention, applying the method for lowering the friction coefficient of a hydrogel or a composite material containing a hydrogel, namely incorporating co-polymeric compound as described herein in any of the respective embodiments in a hydrogel as described herein in any of the respective embodiments, reduces the friction coefficient of the neat hydrogel by a factor of at least 1.5, or at least 2, or at least 3, or at least 5, or at least 10, or at least 20, or at least 50, or at least 100, or at least 500, or at least 1000, or more, including any intermediate values and subranges therebetween.

According to some embodiments of the present invention, the dynamic friction coefficient in aqueous medium of the hydrogel or composite material containing a hydrogel having the co-polymeric compound incorporated therein is reduced by a factor of at least 1.5, or at least 2, or at least 3, or at least 5, or at least 10, or at least 20, or at least 50, or at least 100, or at least 500, or at least 1000, or more, relative to the friction coefficient of the hydrogel not having the co-polymeric compound incorporated therein, including any intermediate values and subranges therebetween.

In some embodiments, the low friction coefficient of the composition or composite material as described in herein in any of the embodiments is maintained under a wide range of loads and in various surface-to-surface combinations, including a gel-to-gel configuration, a gel-to-metal configuration, and even a gel-to-polyethylene, as these are described in detail herein and in the Examples section hereinbelow.

In some embodiments, the low friction coefficient of the composition or composite material as described in herein in any of the embodiments is maintained under a wide range of loads and in gel-to-hydrophobic surface, as described herein in any of the respective embodiments.

The reduction in friction coefficient and the durability of these compositions under continuous sliding motion under loads is maintained for extended duration of time.

According to some embodiments of the present invention, the method further comprises dehydrating the hydrogel or composite material containing a hydrogel and rehydrating the hydrogel, as described herein.

The reduction in friction coefficient is maintained even after the hydrogel/co-polymeric compound composition or composite material has been dehydrated.

According to some embodiments of the present invention, the low friction coefficient of the composition or composite material presented herein, is substantially maintained after at least one dehydration-rehydration cycle, as described herein in any of the respective embodiments.

A co-polymeric compound, a composition or composite material as described herein in any of the embodiments and any combination thereof, and optionally a lipid bi-layer or liposome comprising the co-polymeric compound, as described herein, is useful for lubricating a surface, for example, a surface coated by a co-polymeric compound, a composition or composite material as described herein, and/or a lipid bi-layer or liposome comprising the co-polymeric compound, and/or contacted with a co-polymeric compound, or a composition or composite material comprising the co-polymeric compounds as described herein in any of the embodiments and any combination thereof, or a lipid bi-layer or liposome comprising the co-polymeric compound, as described herein.

According to some of any of the embodiments described herein, the co-polymeric compound, or the composition or composite material as described herein in any of the embodiments and any combination thereof, and optionally a lipid bi-layer or liposome comprising the co-polymeric compound, is for use in lubricating objects (e.g., inanimate objects such as stents, catheters, condoms).

In some of any of the embodiments described herein, at least a portion (e.g., at least 10%, or at least 25%, or at least 50%, or at least 75%, or at least 90%, or at least 95%, or essentially all) of a surface of the object is made of a hard metal surface, a hydrophobic surface, and/or a soft hydrogel surface, as those are described herein in any of the respective embodiments.

In preferred embodiments, the object comprises in at least a portion thereof a hydrophobic surface as described herein in any of the respective embodiments. Non-limiting examples of objects that comprise a hydrophobic surface include medical devices (e.g., stents, catheters, implants, condoms), laboratory equipment (e.g., pipettes, beakers, flasks), cookware (e.g., non-stick pans, baking sheets), automotive parts (e.g., hydrophobic coatings on windshields, metal parts for rust prevention), electronics (e.g., waterproof casings, circuit boards), construction materials (e.g., hydrophobic tiles, insulation materials, tents), textiles (e.g., water-resistant fabrics, outdoor gear, jackets, tents), packaging materials (e.g., food packaging, containers), industrial machinery components (e.g., gears, bearings, seals), aerospace components (e.g., hydrophobic coatings on aircraft surfaces, landing gear), recreational gear (e.g., tents, sleeping bags, backpacks), storage containers (e.g., inner walls of a box, a can for organic products susceptible to spoilage, such as food and drink), conduits (e.g., tubes, pipes, water pipes for organic products or to prevent biofouling), agricultural and food processing machinery (e.g., surfaces of machinery, kitchen surfaces, water-purification equipment), surfaces exposed to moisture (e.g., bathroom walls, water system components, outer surfaces of housing exposed to rain, surfaces in the vicinity of water leakage), and furniture (e.g., chairs, tables). Additional objects which are used in moisture-prone environments are also contemplated.

According to an aspect of some embodiments of the invention, there is provided a lubricant composition comprising the co-polymeric compound, or the composition or composite material according to any of the embodiments described herein, or, optionally a lipid bi-layer or liposome comprising the co-polymeric compound as described herein.

Herein, a “lubricant composition” refers to a composition intended for reducing a friction coefficient of a surface (e.g., according to a method described herein).

In some embodiments, the lubricant composition comprises a carrier. The carrier may optionally be a liquid carrier. In some embodiments, the carrier comprises an aqueous liquid.

In some embodiments, the lubricant composition (or any other composition described herein comprising the co-polymeric compound, or the composition or composite material as described herein, or optionally a lipid bi-layer or liposome comprising the co-polymeric compound) further comprises a water-soluble polymer, optionally as part of the carrier.

As used herein, the phrase “water-soluble polymer” encompasses polymers having a solubility of at least 1 gram per liter in an aqueous (e.g., water) environment at pH 7 (at 25° C.).

In some embodiments of any of the embodiments described herein, the water-soluble polymer has a solubility of at least 2 grams per liter (under the abovementioned conditions). In some embodiments, the solubility is at least 5 grams per liter. In some embodiments, the solubility is at least 10 grams per liter. In some embodiments, the solubility is at least 20 grams per liter. In some embodiments, the solubility is at least 50 grams per liter. In some embodiments, the solubility is at least 100 grams per liter.

The water-soluble polymer(s) according to any of the embodiments described herein may comprise at least one ionic polymer and/or at least one non-ionic polymer which are water-soluble as defined herein.

As used herein, the phrase “non-ionic polymer” refers to a polymer which does not have a charged group.

Examples of suitable non-ionic water-soluble polymers include, without limitation, polyvinylpyrrolidone (also referred to herein interchangeably as povidone and/or PVP) and polyethylene oxide (also referred to herein interchangeably as PEO, PEG and/or polyethylene glycol).

As used herein, the phrase “ionic polymer” refers to polymers having at least one charged group, and encompasses polymers having a net negative charge (also referred to herein as “anionic polymers”), polymers having a net positive charge (also referred to herein as “cationic polymers”), and polymers having no net charge (also referred to herein as “zwitterionic polymers”), in an aqueous (e.g., water) environment at pH 7.

Herein throughout, the phrase “charged group” refers to any functional group (e.g., a functional group described herein) which is ionic (as defined herein), including, for example, amine, carboxylic acid, sulfate, sulfonate, phosphate and phosphonate. Thus, each electric charge in a moiety or molecule is associated with one charged group, although a single charged group (e.g., non-substituted phosphate) may be associated with more than one electric charge of the same sign (e.g., a dianion, a dication).

Herein throughout, the term “ionic” refers to the presence of an electric charge on at least one atom in a moiety and/or molecule (in at least 50% of moieties and/or molecules in a population) in an aqueous medium (e.g., water) at pH 7. The electric charge may be negative (anionic) or positive (cationic). If more than one electric charge is present, the electric charges may be negative (anionic) and/or positive (cationic), for example, both a negative and a positive charge may be present (zwitterionic).

Examples of ionic polymers include, without limitation, ionic polysaccharides, such as hyaluronic acid, chondroitin sulfate, alginic acid, xanthan gum, chitosan and N-alkyl chitosan derivatives.

According to another aspect of embodiments described herein, there is provided a method of lowering a friction coefficient of a surface of a substrate, the method comprising applying to the surface the co-polymeric compound, or the composition or composite material according to any of the respective embodiments described herein, or optionally a lipid bi-layer or liposome comprising the co-polymeric compound, as described herein. In some embodiments, the method is effected by contacting the surface with the co-polymeric compound, or the composition or composite material according to any of the respective embodiments described herein, or optionally a lipid bi-layer or liposome comprising the co-polymeric compound as described herein, or contacting the surface with a lubricant composition according to any of the respective embodiments described herein, which may further comprise a carrier.

In some of any one of the embodiments described herein, lowering the friction coefficient is under aqueous or non-aqueous conditions. In some such embodiments, the substrate is a physiological substrate as defined herein. In some such embodiments, the substrate is a medical device (e.g. an implantable medical device, e.g., as described herein).

In some of any one of the embodiments described herein which relate to a lubrication, according to any one of the aspects described herein, the lubrication is optionally effected according to any of the embodiments described in WO 2015/193887 and/or WO 2015/193888, which are incorporated herein by reference (especially in respect to methods and compositions for lubricating a surface), with the proviso that at least a portion of the co-polymeric compound, or the composition or composite material, or optionally a lipid bi-layer or liposome comprising the co-polymeric compound, is in accordance with any of the embodiments described herein.

In some embodiments, the method further comprises contacting the surface with a water-soluble polymer (e.g., according to any of the respective embodiments described herein), optionally prior to and/or concomitantly with contacting the surface with the co-polymeric compound, or the composition or composite material as described herein, or optionally with a lipid bi-layer or liposome comprising the co-polymeric compound as described herein. In some embodiments, the method is effected by contacting the surface with a co-polymeric compound, or a composition or composite material as described herein in any of the respective embodiments, or optionally with a lipid bi-layer or liposome comprising the co-polymeric compound, as described herein, and the water-soluble polymer (optionally a lubricant composition comprising the water-soluble polymer according to any of the respective embodiments described herein), optionally in combination with an aqueous liquid.

In some of any one of the embodiments described herein which relate to a method and/or lubrication composition for reducing a friction coefficient of a surface, the surface is a hard metal surface (e.g., stainless steel surface). In alternative embodiments, the surface is a hydrophobic surface (e.g., a polyethylene surface). In alternative embodiments, the surface is a soft hydrogel surface (e.g., a pHEMA surface). In alternative embodiments, the surface is a hydrophilic surface.

Example of hydrophobic hydrogel surface is PDMS, and of hydrophilic hydrogel surface is polyvinyl alcohol (PVA).

Herein, the phrase “hard metal surface” encompasses a surface that comprises, in at least a portion thereof (e.g., at least 10%, or at least 25%, or at least 50%, or at least 75%, or at least 90%, or more) a metal specie. Non-limiting examples of metal species include metal particles, metal oxides (e.g., anodized surfaces), metal carbides, metal nitrides, metal sulfides, metal phosphides, metal films, metal nanostructures (e.g., metal NPs, metal nanowires), metal coatings, and alloys (e.g., stainless steel surface).

Herein, the phrase “soft hydrogel surface” encompasses a surface which is or comprises a hydrogel as described herein. Non-limiting examples of soft hydrogel surfaces include hydrogels made from at least hydrogel-forming agent as described herein, such as pHEA, pAAm, pMAAm, pAAc, pNIPAM, and PEG-based hydrogels, and any oligomer, polymer and/or copolymer thereof.

Exemplary hydrophilic surfaces include surfaces made of hydrophilic or amphiphilic polymers or co-polymeric, including, for example, polyvinyl alcohol (PVA), polyethylene Glycol (PEG), polyacrylamides (PAM), polyacrylic Acid (PAA), poly(N-vinylpyrrolidone) (PVP), cellulose and cellulose derivatives (e.g., hydroxyethyl Cellulose), and any co-polymers and combinations thereof.

Herein, the phrase “hydrophobic surface” encompasses a surface that is hydrophobic and/or is or comprises a hydrophobic material, as those are described herein.

Non-limiting examples of hydrophobic materials include hydrophobic polymers and non-polymeric hydrophobic materials.

Exemplary non-polymeric hydrophobic materials which are usable as surfaces include, but are not limited to, waxes, fatty acids, graphene, ceramic coatings (e.g., silicon carbide, alumina), alkylated surfaces (e.g., silanized glass, alkylated cellulose), and metals coated with hydrophobic agents (e.g., silane-coated aluminum).

Exemplary hydrophobic polymers which are usable as surfaces include, but are not limited to, silicones (e.g., PDMS), fluoropolymers such as polytetrafluoroethylene (PTFE; Teflon®) or polyvinylidene fluoride (PVDF), polyethylenes, polypropylenes, polystyrenes, polyvinyl chloride (PVC), poly(methyl methacrylate) (PMMA), polycarbonates (PC), polyurethanes, polyimides, and non-polymeric materials coated with hydrophobic polymers (e.g., fluoropolymer-coated stainless steel).

In some of any one of the embodiments described herein which relate to a method and/or lubrication composition for reducing a friction coefficient of a surface, the surface is a contact lens surface.

In some of any one of the embodiments described herein which relate to a contact lens, according to any one of the aspects described herein, the contact lens comprises a hydrogel surface. In some embodiments, the contact lens comprises a hydrogel surface and a rigid center. In some embodiments, the contact lens consists essentially of a hydrogel. In some such embodiments, the hydrogel comprise any material known in the art for use in contact lens hydrogels. Examples of such hydrogel materials include, without limitation, alphafilcon A, asmofilcon A, balafilcon A, bufilcon A, comfilcon A, crofilcon, deltafilcon A, dimefilcon, droxifilcon A, enfilcon A, etafilcon A, galyfilcon A, hefilcon A, hefilcon B, hilafilcon A, hilafilcon B, hioxifilcon A, hioxifilcon D, isofilcon, lidofilcon A, lidofilcon B, lotrafilcon B, mafilcon, methafilcon A, methafilcon B, narafilcon A, narafilcon B, ocufilcon A, ocufilcon B, ofilcon A, omafilcon A, perfilcon, phemfilcon A, polymacon, scafilcon A, senofilcon A, surfilcon, tefilcon, tetrafilcon A, tetrafilcon B, vifilcon A, and xylofilcon A.

In some embodiments of any one of the embodiments described herein, the hydrogel comprises a polymer consisting of poly(2-hydroxyethyl methacrylate) and/or a silicone. In some embodiments, the polymer comprises a silicone. Such polymers may optionally comprise small amounts of additional monomers (e.g., crosslinking monomers) copolymerized with the 2-hydroxyethyl methacrylate or silicone monomer. For example, 2-hydroxyethyl methacrylate may optionally be copolymerized with vinyl pyrrolidone, methyl methacrylate, methacrylic acid (an anionic monomer), ethylene glycol dimethacrylate (a crosslinking monomer) and/or 3-(ethyldimethyl-ammonium) propyl methacrylamide (a cationic monomer) in a contact lens hydrogel.

According to some of any of the embodiments described herein, the co-polymeric compound, the liposome, or the composition or composite material as described herein in any of the embodiments and any combination thereof, is for use in lubricating a biological tissue (e.g., when applied to an external surface of the tissue via, for example, adsorption from solution).

In some of any of the embodiments described herein relating to a method and/or lubrication composition for reducing a friction coefficient of a surface of a substrate, the surface is a physiological surface, and a carrier used with the co-polymeric compound, or the composition or composite material as described herein (e.g., in a lubricant composition according to any of the respective embodiments described herein) is a physiologically acceptable carrier, as described herein.

In some embodiments, a surface for which a friction coefficient is reduced according to any of the respective embodiments described herein, is an articular surface of a synovial joint.

In some embodiments, the method of reducing a friction coefficient of a surface is for use in the treatment of a synovial joint disorder associated with an increased friction coefficient of an articular surface in the synovial joint.

In some embodiments of any of the embodiments described herein, the co-polymeric compound, or the composition or composite material as described herein in any of the respective embodiments and any combination thereof, or of a lipid bi-layer or liposome made of the co-polymeric compound as described herein, is for use in the treatment of a synovial joint disorder associated with an increased friction coefficient of an articular surface in the synovial joint.

According to another aspect of embodiments described herein, there is provided a use of a co-polymeric compounds, or a composition or composite material according to any of the respective embodiments described herein, or optionally of a lipid bi-layer or liposome made of the co-polymeric compound as described herein, in the manufacture of a medicament for treating a synovial joint disorder associated with an increased friction coefficient of an articular surface in the synovial joint.

Examples of synovial joint disorders associated with an increased friction coefficient of an articular surface, and treatable according to some of any of the embodiments described herein, include, without limitation, arthritis, traumatic joint injury, locked joint (also known in the art as joint locking), and joint injury associated with surgery.

In some embodiments, the arthritis is osteoarthritis, rheumatoid arthritis and/or psoriatic arthritis.

In some embodiments, the locked joint is associated with osteochondritis dissecans and/or synovial osteochondromatosis.

The joint injury associated with surgery described herein may optionally be associated with surgery which directly inflicts damage on an articular surface (e.g., by incision), and/or surgery which damages an articular surface only indirectly. For example, surgery which repairs or otherwise affects tissue in the vicinity of the joint (e.g., ligaments and/or menisci) may be associated with joint injury due to altered mechanics in the joint.

The traumatic joint injury described herein may optionally be injury caused directly by trauma (e.g., inflicted at the time of the trauma) and/or injury caused by previous trauma (e.g., a post-traumatic injury which develops sometime after the trauma).

According to some of any of the embodiments described herein, the co-polymeric compound, or the composition or composite material as described herein in any of the embodiments and any combination thereof, or optionally or of a lipid bi-layer or liposome made of the co-polymeric compound as described herein, is for use in the treatment and/or alleviation of osteoarthritis (OA) (e.g., as biolubricants for intra-articular administration).

### Pharmaceutical Compositions:

The co-polymeric compound, or the composition or composite material as described herein in any of the respective embodiment, or a lipid bi-layer or liposome made of the co-polymeric compound as described herein, may optionally be administered as part of a composition (e.g., a solution) that comprises a physiologically acceptable carrier, for example an aqueous carrier which is a physiologically acceptable carrier.

Herein throughout, the term “physiologically acceptable carrier” refers to a carrier or a diluent that does not cause significant irritation to a subject upon administration in the intended manner, and does not abrogate the activity and properties of the co-polymeric compound in the composition (e.g., their ability to reduce a friction coefficient of a surface, as described herein in any one of the respective embodiments). Examples, without limitations, of carriers are: propylene glycol, saline, emulsions and mixtures of organic solvents with water, as well as solid (e.g., powdered) and gaseous carriers.

Techniques for formulation and administration of compounds may be found in “Remington's Pharmaceutical Sciences” Mack Publishing Co., Easton, PA, latest edition, which is incorporated herein by reference.

Solutions according to any one of the embodiments of the present invention may be manufactured by processes well known in the art, e.g., by means of conventional mixing or dissolving processes.

Solutions for use in accordance with the present invention thus may be formulated in conventional manner using one or more physiologically acceptable carriers, which facilitate processing of the co-polymeric compounds or the composition or composite material into preparations which can be used pharmaceutically. Proper formulation is dependent upon the route of administration chosen.

For injection, the co-polymeric compound, the liposome, or the composition or composite material as described herein may be formulated in aqueous solutions, preferably in physiologically compatible buffers such as Hank's solution, Ringer's solution, histidine buffer, or physiological saline buffer with or without organic solvents such as propylene glycol, polyethylene glycol.

The co-polymeric compound, the liposome, or the composition or composite material as described herein may be formulated for parenteral administration, e.g., by bolus injection or continuous infusion. Formulations for injection may be presented in unit dosage form, e.g., in ampoules or in multi-dose containers with optionally, an added preservative. The compositions may be suspensions, solutions or emulsions in oily or aqueous vehicles, and may contain formulatory agents such as suspending, stabilizing and/or dispersing agents.

The co-polymeric compound, or the composition or composite material as described herein, or a lipid bi-layer or liposome made of the co-polymeric compound as described herein, may be formulated as an aqueous solution per se. Additionally, the solution may be in the form of a suspension and/or emulsions (e.g., the aqueous phase of a suspension or water-in-oil, oil-in-water or water-in-oil-in-oil emulsion), for example, in order to increase the viscosity of the formulation. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the co-polymeric compound, the liposome, or the composition or composite material as described herein, for example, to allow for the preparation of highly concentrated solutions.

In some embodiments, the co-polymeric compound, the liposome, or the composition or composite material as described herein may be in powder form for constitution with a suitable vehicle, e.g., sterile, pyrogen-free water, before use.

The solutions may be formulated wherein the co-polymeric compound, the liposome, or the composition or composite material as described herein in any of the respective embodiments are contained in an amount effective to achieve the intended purpose, for example, an amount effective to prevent, alleviate or ameliorate symptoms of a disorder in the subject being treated.

The dosage may vary depending upon the dosage form employed, the route of administration utilized, and the location of administration (e.g., the volume and/or surface of the region contacted with the co-polymeric compound, the liposome, or the composition or composite material as described herein).

The amount of a composition to be administered will, of course, be dependent on the subject being treated, the severity of the affliction, the manner of administration, the judgment of the prescribing physician, etc.

Compositions (e.g., solutions) according to some of any of the embodiments described herein may, if desired, be presented in a pack or dispenser device, such as an FDA (the U.S. Food and Drug Administration) approved kit, which may contain one or more unit dosage forms containing the active ingredient(s) (e.g., the co-polymeric compounds or the composition or composite material as described herein). The pack may, for example, comprise metal or plastic foil, such as, but not limited to a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising the co-polymeric compound, the liposome, or the composition or composite material as described herein (optionally with a water-soluble polymer described herein), as described herein in any one of the respective embodiments, formulated in a physiologically acceptable carrier may also be prepared, placed in an appropriate container, and labeled for treatment of an indicated condition or diagnosis, as is detailed herein.

### Inhibition of Biofilm Formation and Biofouling:

A co-polymeric compound, or a composition or composite material as described herein, or optionally a lipid bi-layer or liposome made of the co-polymeric compound as described herein, may optionally or in addition be useful for inhibiting adhesion, biofouling and/or biofilm formation on a surface, for example, a surface coated by a composition or composite material as described herein, and/or contacted with a composition or composite material as described herein.

According to an aspect of some embodiments of the invention, there is provided a method of inhibiting adsorption of a biofouling-promoting agent on a surface of a substrate. The method, according to some embodiments of the present invention, is effected by contacting the substrate with a co-polymeric compound, or a composition or composite material according to any of the respective embodiments, or optionally with a lipid bi-layer or liposome made of the co-polymeric compound as described herein.

The term “biofouling-promoting agent”, as used herein throughout, refers to an agent whose presence facilitates formation and/or participates in formation of a biofilm (as defined herein) on a substrate surface. An agent is considered to facilitate formation of a biofilm on a substrate surface when a presence of the agent enhances formation of a biofilm on a substrate surface as compared to formation of a biofilm on the same substrate surface in an absence of the agent. An agent is considered to participate in formation of a biofilm on a substrate surface when the biofilm formed on the surface comprises the agent as a portion of the biofilm.

In some embodiments of any of the embodiments described herein, an agent is identified as a biofouling-promoting agent by comparing growth (e.g., over the course of 1, 2, 3, 4, 5, 6 or 7 days) of a biofilm (e.g., P. aeruginosa) on the surface in the presence of an aqueous liquid (e.g., water or broth, optionally at 37° C.) and the agent, to growth of a biofilm (under the same conditions) on the surface in the presence of the same aqueous liquid (e.g., water or broth) without the agent. The agent is optionally mixed within the aqueous liquid, or alternatively, adsorbed onto the surface prior to exposure of the surface to the aqueous liquid. The growth of the biofilm is considered as the biofilm load at the end of the growth period (e.g., 1, 2, 3, 4, 5, 6 or 7 days) minus the initial biofilm load. Optionally, the measurement is performed such that the initial biofilm is substantially zero (e.g., absent or at least undetectable), for example, the microorganism is in a planktonic form, such that growth of the biofilm is considered as the biofilm load at the end of the growth period. In some embodiments of any of the embodiments described herein, biofilm load is defined as an area of the biofilm.

In some embodiments of any of the embodiments described herein, biofilm load is defined as a mass and/or volume of the biofilm.

In some embodiments of any of the embodiments described herein, biofilm load is defined as a number of cells in the biofilm.

The biofilm load may optionally be determined using any technique known in the art for detecting and quantifying an amount of cells and/or microorganisms in a biofilm.

In some of these embodiments, an agent is considered a biofouling-promoting agent if the growth of a biofilm in its presence is at least 10% higher, or at least 20% higher, or at least 50% higher, or at least 100% higher (i.e., two-fold), or more, than the growth of a biofilm in the absence of the agent, including any intermediate values and subranges therebetween.

Examples of biofouling-promoting agents include, without limitation, a biofouling-promoting protein and a biofouling-promoting polysaccharide, that is, any protein or polysaccharide which is a biofouling-promoting agent as defined herein.

In some embodiments of any of the embodiments described herein, the method is considered as being capable of inhibiting adsorption of a biofouling-promoting agent when the method is capable of inhibiting adsorption of a selected biofouling-promoting agent (e.g., the selected agent is considered representative of biofouling-promoting agents in general). In some embodiments, the selected biofouling-promoting agent is a protein. In some embodiments, the selected protein is an antibody which does not exhibit any specific affinity to the substrate (e.g., an anti-IgG antibody).

The term “biofilm”, as used herein throughout, refers to an aggregate of living cells which are stuck to each other and/or immobilized onto a surface as colonies. The cells are frequently embedded within a self-secreted matrix of extracellular polymeric substance (EPS), also referred to as “slime”, which is a polymeric sticky mixture of nucleic acids, proteins and polysaccharides.

In the context of the present embodiments, the living cells forming a biofilm can be cells of a unicellular microorganism, including prokaryotes (e.g., bacteria, archaeal microorganisms) and eukaryotes such as fungi and protists (e.g., algae, euglena, protozoa, dinoflagellates, apicomplexa, trypanosomes, amoebae) and the like; or cells of multicellular organisms, in which case the biofilm can be regarded as a colony of cells (as in the case of the unicellular organisms) or as a lower form of a tissue.

According to some embodiments of any of the embodiments of the present invention, the cells are of microorganism origins, and the biofilm is a biofilm of microorganisms, such as bacteria, archaeal microorganisms, protists and fungi. The cells of a microorganism growing in a biofilm are typically physiologically distinct from cells in the “planktonic” form of the same organism, which by contrast, are single cells that may float or swim in a liquid medium.

The substrate may be any substrate described herein, and encompasses any surface, structure, product or material which can support, harbor or promote the growth of a microorganism. The substrate is optionally a portion of an object (e.g., an article-of-manufacture) which can support, harbor or promote the growth of a microorganism. Such a portion of an object may span only a portion of an area of the object, such that a surface of the substrate represents only a portion of a surface of the object (e.g., a portion most likely to support, harbor or promote the growth of a microorganism); and/or span only a portion of the thickness of the object (e.g., along an axis perpendicular to a surface of the substrate and object), such that the substrate does not include the entire volume of the object lying underneath a surface of the substrate (which may represent the entire surface of the object or only a portion of the surface of the object). Non-limiting examples include the inner walls of a storage container (e.g., a box, a can) and/or conduit (e.g., a tubes, a pipe) for an organic product susceptible to spoilage associated with biofouling, for example, food and/or drink (e.g., a food container, a water pipe), surfaces intended to come into contact with such an organic product (e.g., agricultural and/or food processing machinery, a kitchen surface, water-purification equipment), and surfaces exposed to moisture (e.g., bathroom walls, water system components, outer surfaces of housing exposed to rain, surfaces in the vicinity of water leakage).

In some embodiments, the substrate is a medical device or any other device which is intended for contacting a living tissue, as defined herein.

In some embodiments of any of the embodiments described herein, the inhibition of adsorption described herein is for reducing adhesion of pathogenic microorganisms (e.g., any biofilm-forming microorganism described herein which is potentially pathogenic) to a medical device (e.g., any medical device described herein).

In some embodiments of any of the embodiments described herein, adsorption of the biofouling-promoting agent (any biofouling-promoting agent described herein) on the surface of the substrate subjected to a method described herein (according to any of the respective embodiments) is reduced by at least 10% relative to adsorption on the surface of the substrate in the absence of the co-polymeric compound, the liposome, or the composition or composite material as described herein in any of the respective embodiments and any combination thereof.

In some embodiments, adsorption is reduced by at least 20%, or by at least 30%, or by at least 40%, or by at least 50%, or by at least 60%, or by at least 70%, or by at least 80%, or by at least 90%, relative to adsorption on the surface of the substrate in the absence of the co-polymeric compound, the liposome, or the composition or composite material as described herein in any of the respective embodiments and any combination thereof.

Reduction of an amount of adsorbed biofouling-promoting agent may optionally be determined using any technique known in the art for detecting and quantifying an amount of agent, including, without limitation, using a labeled biofouling-promoting agent (e.g., as exemplified herein in the Examples section). The reduction is optionally measured by contacting each of the aforementioned surfaces (e.g., for 2 hours) with an aqueous solution (optionally comprising phosphate buffer, e.g., 0.1 M phosphate) of the biofouling-promoting agent (e.g., at 37° C. and/or pH 7), followed by repeated rinses to remove non-adsorbed agent (e.g., as exemplified in the examples section herein). The concentration of the biofouling-promoting agent in the aqueous solution is optionally 1 μg/ml or the concentration of a saturated solution of the agent, whichever concentration is lower.

Herein throughout, the term “biofilm-promoting conditions” refers to conditions suitable for formation and growth of a biofilm of a cell (e.g., P. aeruginosa), for example, wherein a surface is in contact (e.g., over the course of 1, 2, 3, 4, 5, 6 or 7 days) with an aqueous liquid (e.g., water or broth, optionally at 37° C.) containing such cells.

In some embodiments of any of the embodiments described herein, biofilm load is defined as an area of the biofilm.

In some embodiments of any of the embodiments described herein, biofilm load is defined as a mass and/or volume of the biofilm.

In some embodiments of any of the embodiments described herein, biofilm load is defined as a number of cells in the biofilm.

The biofilm load may optionally be determined using any technique known in the art for detecting and quantifying an amount of cells and/or microorganisms in a biofilm.

In some embodiments of any of the embodiments described herein, the time period of biofilm formation, after which biofilm load is determined, is in determined in accordance with the biofilm load, for example, the time period being a time period after which a biofilm covers 100%, 50%, or any other pre-determined percentage of the area of the substrate in the absence of inhibition of biofilm formation by contact with the co-polymeric compound, the liposome, or the composition or composite material as described herein. For example, if a biofilm grows to cover 50% of a surface in the absence of biofilm formation inhibition, and during the same a time period, a biofilm grows to cover 30% of a surface in the presence of biofilm formation inhibition, then inhibition of biofilm formation may be considered to result in a reduction of 40% (i.e., (50%-30%)/50%) in biofilm formation.

Herein, the phrase “upon contact with the agent” means that in addition to the biofilm-promoting conditions, the agent is also present (e.g., in the aqueous liquid containing the cells).

According to an aspect of some embodiments of the invention, there is provided a method of inhibiting biofilm formation on a surface of a substrate as defined herein in any embodiment and any combination of embodiments, the method comprising contacting the substrate with a co-polymeric compound, a liposome, or a composition or composite material as described herein in any of the respective embodiments and any combination thereof.

In some embodiments, “inhibiting biofilm formation” refers to the prevention of formation of a biofilm; and/or to a reduction in the rate of buildup of a biofilm; and/or to a reduction in the mass of a biofilm, the area or the volume of the biofilm, or in the number of cells forming the biofilm.

In some embodiments of any of the embodiments described herein, the inhibition of adsorption described herein is for reducing adhesion of pathogenic microorganisms (e.g., any biofilm-forming microorganism described herein which is potentially pathogenic) to a medical device. Such a reduction may result in inhibiting biofilm formation, as defined in some embodiments herein.

In some embodiments of any of the embodiments described herein, biofilm formation on the surface of the substrate subjected to a method described herein (according to any of the respective embodiments) is reduced by at least 10% relative to biofilm formation on the surface of the substrate in the absence of the co-polymeric compound, the liposome, or the composition or composite material as described herein. In some embodiments, biofilm formation is reduced by at least 20%. In some embodiments, biofilm formation is reduced by at least 30%. In some embodiments, biofilm formation is reduced by at least 40%. In some embodiments, biofilm formation is reduced by at least 50%. In some embodiments, biofilm formation is reduced by at least 60%. In some embodiments, biofilm formation is reduced by at least 70%. In some embodiments, biofilm formation is reduced by at least 80%. In some embodiments, biofilm formation is reduced by at least 90%.

The reduction in biofilm formation is optionally determined by measuring a biofilm load (in accordance with any of the respective embodiments described herein) for a biofilm of a cell (e.g., P. aeruginosa) on each surface after being subjected to biofouling-promoting conditions, as defined herein (e.g., over the course of 1, 2, 3, 4, 5, 6 or 7 days, or any other time period as described herein).

Any of the embodiments described herein relating to inhibition of biofilm formation and/or biofouling may optionally be effected by a composition which is essentially the same as a lubricant composition according to any of the respective embodiments described herein (although optionally identified for inhibition of biofilm formation and/or biofouling rather than for lubrication).

In some of any one of the embodiments described herein which relate to a inhibition of adhesion, biofilm formation and/or biofouling, according to any one of the aspects described herein, the inhibition is optionally effected according to any of the embodiments described in Israel Patent Application No. 234929 and/or WO 2016/051413, which are incorporated herein by reference (especially in respect to methods and compositions for inhibiting adhesion, biofilm formation and/or biofouling on a surface), with the proviso that at least a portion of the co-polymeric compounds, the liposome, or the composition or composite material used are in accordance with any of the embodiments described herein.

According to some of any of the embodiments of the invention, inhibition of the adsorption of the biofouling-promoting agent is substantially unchanged after at least one dehydration-rehydration cycle of the hydrogel.

### Articles-of-Manufacturing:

According to an aspect of some embodiments of the invention, there is provided an article-of-manufacturing comprising the co-polymeric composition, or the composition or composite material as described herein in any of the respective embodiments, or optionally a lipid bi-layer or liposome made of the co-polymeric compound as described herein.

Non-limiting examples of article-of-manufacturing include, but are not limited to, an implantable medical device, a drug-delivery system, a solid body, a disc, a fiber, a fabric, a tube, a film, a rod, a ring, a tubular mesh, and any combination thereof.

According to some of any of the embodiments described herein, the co-polymeric composition, or the composition or composite as described herein in any of the respective embodiments, or optionally a lipid bi-layer or liposome made of the co-polymeric compound as described herein, is for use in replacing missing or damaged cartilage in a living organism suffering from a medical condition associated with loss of or damaged cartilage. Non-limiting examples of the medical conditions include skeletal joint replacement or reconstruction, vertebrate replacement or reconstruction, tendon replacement, tissue regeneration, and reduction of tissue irritation by an implantable device.

In any of the embodiments described herein, the substrate may comprise any type of material or combination of different types of material, including inorganic material and/or organic material, in crystalline, amorphous and/or gel (e.g., hydrogel) forms, for example, metal, mineral, ceramic, glass, polymer (e.g., synthetic polymer, biopolymer), plant and/or animal biomass, and combinations thereof.

In some embodiments, the substrate comprises a physiological surface (e.g., a physiological tissue) and/or a surface in contact with and/or intended to come into contact with a physiological surface (e.g., as described herein in any one of the respective embodiments).

In some embodiments of any of the embodiments described herein, the article is a medical device (the substrate being a medical device or portion of a medical device, as described herein). In some embodiments, the medical device is a device designed to come into contact with a part of the body susceptible to infection, such as an internal portion of the body, a mucous membrane and/or a surface of the eye. Examples of such medical devices include, without limitation, catheters, surgical tools and implants (which are for coming into contact with an internal portion of the body) and contact lenses (which are for contacting a surface of the eye).

As used herein throughout, the phrase “medical device” includes any material or device that is used on, in, or through a subject's body, for example, in the course of medical treatment (e.g., for a disease or injury). The subject may be human or a non-human animal, such that the phrase “medical device” encompasses veterinary devices. Medical devices include, but are not limited to, such items as medical implants (including permanent implants and transient implants), wound care devices, medical devices for drug delivery, contact lenses and body cavity and personal protection devices. The medical implants include, but are not limited to, catheters (e.g., urinary catheters, intravascular catheters), injection ports, intubation equipment, dialysis shunts, wound drain tubes, skin sutures, vascular grafts, implantable meshes, intraocular devices, heart valves, and the like. Wound care devices include, but are not limited to, general wound dressings, biologic graft materials, tape closures and dressings, and surgical incise drapes. Medical devices for drug delivery include, but are not limited to, needles, drug delivery skin patches, drug delivery mucosal patches and medical sponges. Body cavity and personal protection devices, include, but are not limited to, tampons, sponges, surgical and examination gloves, and toothbrushes. Birth control devices include, but are not limited to, intrauterine devices (IUDs), diaphragms and condoms.

Exemplary articles include the following:
Medical devices such as, but not limited to, pacemakers, heart valves, replacement joints, catheters, catheter access ports, dialysis tubing, gastric bands, shunts, screw plates, artificial spinal disc replacements, internal implantable defibrillators, cardiac resynchronization therapy devices, implantable cardiac monitors, mitral valve ring repair devices, left ventricular assist devices (LVADs), artificial hearts, implantable infusion pumps, implantable insulin pumps, stents, implantable neurostimulators, maxillofacial implants, dental implants, and the like;
Packages or containers, for example, food packages and containers, beverage packages and containers, medical device packages, agricultural packages and containers (of agrochemicals), blood sample or other biological sample packages and containers, and any other packages or containers of various articles;
Food packages such as packages of dairy products and/or containers for storage or transportation of dairy products;
Milk storage and processing devices such as, but not limited to, containers, storage tanks, raw milk holding equipment, dairy processing operations conveyer belts, tube walls, gaskets, rubber seals, stainless steel coupons, piping systems, filling machine, silo tanks, heat exchangers, post-pasteurization equipment, pumps, valves, separators, and spray devices;
Energy harvesting device, for example, a microelectronic device, a microelectromechanical device, a photovoltaic device and the like;
Microfluidic devices, for example, micro-pumps or micro valves and the like;
Sealing parts, for example, O rings, and the like;
Articles having a corrodible surface;
Agricultural devices, as, for example, described herein;
Textiles, for example, tough cottons;
Fuel transportation devices;
Construction elements, such as, but not limited to, paints, walls, windows, door handles, and the like;
Elements of water treatment systems (such as for containing and/or transporting and/or treating aqueous media or water), devices, containers, filters, tubes, solutions and gases and the like; and
Elements of organic waste treatment systems (such as for containing and/or disposing and/or transporting and/or treating organic waste), devices, containers, filters, tubes, solutions and gases and the like.

According to some of any of the embodiments described herein, the article-of-manufacturing comprises a catheter having applied on at least a portion thereof a co-polymeric compound or a composition or composite material, as described herein in any of the respective embodiments and any combination thereof.

According to some of any of the embodiments described herein, the co-polymeric compound, or the composition or composite material, or optionally a lipid bi-layer or liposome made of the co-polymeric compound as described herein, reduces wear and surface damage.

### Lipid Bilayers and Liposomes:

According to an aspect of some embodiments of the invention, there is provided a lipid bilayer (referred to herein interchangeably as simply a “bilayer”) comprising a polymeric compound according to any of the respective embodiments described herein. In some such embodiments, the bilayer further comprises at least one bilayer-forming lipid in addition to the co-polymeric compound.

According to an aspect of some embodiments of the invention, there is provided a lipid bilayer that comprises a co-polymeric compound as described herein in any of the respective embodiments and any combination thereof and one or more bilayer-forming lipid(s), as described herein in any of the respective embodiments.

According to these embodiments, a lipid bilayer or a liposome can be formed when one of E1 and E2 terminal groups is a lipid moiety as described herein in any of the respective embodiments and any combination thereof. According to these embodiments, a total sum of n+m is lower than 1,000 or lower than 500, or lower than 300, or lower than 200, or lower than 100, and can range, for example, from 2 to 1,000 or from 5 to 1,000, or from 10 to 1,000, or from, 50 to 1,000, or from 2 to 100, or from 5 to 100, or from 10 to 100, including any intermediate values and subranges therebetween.

Herein, the term phrase “bilayer-forming lipid” encompasses any compound in which a bilayer may form from a pure aqueous solution of the compound, the bilayer comprising two parallel layers of molecules of the compound (referred to as a “lipid”). It is to be appreciated that the co-polymeric compound described herein in any of the embodiments may optionally comprise a bilayer-forming lipid which can form a bilayer per se or in combination with one or more additional bilayer-forming lipids.

Typically, the bilayer comprises relatively polar moieties of the lipid at the two surfaces of the bilayer, which may optionally comprise an interface with the aqueous solution and/or an interface with a solid surface; and relatively hydrophobic moieties of the lipid at the interior of the bilayer, at an interface between the two layers of lipid molecules which form the bilayer.

Examples of bilayer-forming lipids include glycerophospholipids (e.g., a glycerophospholipid according to any of the respective embodiments described herein). It is to be appreciated that the polymeric compound described herein may optionally be a bilayer-forming lipid which can form a bilayer per se or in combination with one or more additional bilayer-forming lipids. Exemplary glycerophospholipids include phosphatidyl ethanolamine, phosphatidyl serine, phosphatidyl glycerol, and phosphatidyl inositol.

In some embodiments of any one of the embodiments described herein, the bilayer-forming lipid comprises at least one charged group (e.g., one or more negatively charged groups and/or one or more positively charged groups).

In some embodiments, the bilayer-forming lipid is zwitterionic; comprising both (e.g., an equal number of) negatively charged and positively charged groups (e.g., one of each).

In some embodiments of any of the embodiments described herein relating to a bilayer, a molar ratio of the bilayer-forming lipid (comprised in addition to the co-polymeric compound of the present embodiments) and the co-polymeric compound in the bilayer is in a range of from 5:1 to 5,000:1 (bilayer-forming lipid: polymeric compound), optionally in a range of from 10:1 to 2,500:1, optionally in a range of from 25:1 to 1,000:1, and optionally in a range of from 50:1 to 500:1.

In some embodiments of any of the embodiments described herein relating to a bilayer, a polymeric moiety in the bilayer comprises a lipid moiety represented by the variables E₁ or E₂ in formula I (according to any of the respective embodiments described herein) which comprises a moiety of a bilayer-forming lipid (e.g., a glycerophospholipid) which is comprised by the bilayer in addition to the polymeric moiety or which is closely related to a bilayer-forming lipid comprises by the bilayer, for example, wherein the lipid moiety comprises a glycerophospholipid moiety and the bilayer comprises another glycerophospholipid as a bilayer-forming lipid (e.g., optionally wherein fatty acid moieties in the glycerophospholipid moiety have about the same length as fatty acid moieties in the bilayer-forming lipid, and optionally wherein the fatty acid moieties in the glycerophospholipid moiety are substantially the same as the fatty acid moiety in the bilayer-forming lipid).

Without being bound by any particular theory, it is believed that similarity between a lipid moiety of a polymeric moiety and a bilayer-forming lipid facilitates anchorage of the lipid moiety of the polymeric moiety in a bilayer comprising the bilayer-forming lipid.

The bilayer according to some of any of the embodiments described herein may optionally be closed upon itself (e.g., such that the bilayer has no edges), thereby forming an inner volume separated by the bilayer from the surrounding environment, which is referred to herein and in the art as a “liposome”. Alternatively or additionally, the bilayer may be open-faced and/or with edges.

According to an aspect of some embodiments of the invention, there is provided a liposome comprising at least one lipid bilayer according to any of the respective embodiments described herein.

A liposome may optionally comprise a single bilayer or a plurality of bilayers (each bilayer optionally independently forming a closed vesicle) comprising, for example, concentric bilayer vesicles and/or a plurality of separate bilayer vesicles encompassed by the same bilayer vesicle.

According to an aspect of some embodiments of the invention, there is provided a liposome comprising a co-polymeric compound as described herein in any of the respective embodiments and any combination thereof and a bilayer-forming lipid according to any of the respective embodiments described herein.

As used herein and in the art, the term “liposome” refers to an artificially prepared vesicle comprising a bilayer composed of molecules of an amphiphilic lipid. In an aqueous medium, the bilayer is typically configured such that hydrophilic moieties of the amphiphilic lipid are exposed to the medium at both surfaces of the bilayer, whereas lipophilic moieties of the lipid are located in the internal portion of the bilayer, and therefore less exposed to the medium. Examples of liposomes which may be used in any one of the embodiments described herein include, without limitation, small unilamellar vesicles, large unilamellar vesicles, and multilamellar vesicles.

According to some of any of the embodiments described herein, the liposomes are characterized by a diameter (e.g., an average diameter) in a range of from 1 to 1000 nm, or from 10 to 750 nm, or from 100 to 750 nm, including any intermediate values and subranges therebetween.

A liposome may optionally comprise a single bilayer or a plurality of bilayers (each bilayer optionally independently forming a closed vesicle) comprising, for example, concentric bilayer vesicles and/or a plurality of separate bilayer vesicles encompassed by the same bilayer vesicle.

A liposome according to any of the respective embodiments described herein may be approximately spherical in shape or may have any alternative shape, such as an elongated tube and/or a flattened (e.g., sheet-like) shape. In some embodiments of any of the embodiments described herein relating to a liposome, the liposome further comprises at least one functional moiety or agent bound to, or associated with, a surface of the liposome and/or within a lipid bilayer and/or core of the liposome (e.g., within the liposome bilayer and/or enveloped by the liposome bilayer). In some embodiments, a functional moiety is covalently attached to a liposome. Such attachment may be obtained in some embodiments by using techniques known in the art (e.g., amide bond formation).

Examples of functional moieties and agents suitable for inclusion in a liposome include, without limitation, a therapeutically active agent or a moiety of a therapeutically active agent (e.g., wherein the active agent is releasable upon cleavage of the moiety), a labeling moiety or agent, and/or a targeting moiety or targeting agent (e.g., a targeting moiety or agent on a surface of the liposome).

As used herein, the phrase “therapeutically active agent” describes a chemical substance, which exhibit a therapeutic activity when administered to a subject. Non-limiting examples of therapeutically active agents that can be beneficially used in embodiments of the present invention include, without limitation, one or more of agonist agents (e.g., dopamine agonists like pramipexole), amino acid agents (e.g., glutamine), analgesic agents (e.g., acetaminophen), antagonist agents (e.g., beta-blockers such as propranolol), antibiotic agents (e.g., penicillin), antibody agents (e.g., rituximab), antidepressant agents (e.g., fluoxetine), antigen agents (e.g., hepatitis B antigen), anti-histamine agents (e.g., diphenhydramine), anti-hypertensive agents (e.g., losartan), anti-inflammatory drugs (e.g., ibuprofen), anti-metabolic agents (e.g., methotrexate), antimicrobial agents (e.g., amoxicillin), antioxidant agents (e.g., vitamin E), anti-proliferative drugs (e.g., sirolimus), antisense agents (e.g., mipomersen), chemotherapeutic drugs (e.g., doxorubicin), co-factors (e.g., coenzyme Q10), cytokines (e.g., interleukin-2), drugs (e.g., aspirin), enzymes (e.g., trypsin), growth factors (e.g., epidermal growth factor), heparins (e.g., enoxaparin), hormones (e.g., insulin), immunoglobulins (e.g., IgG), inhibitors (e.g., protease inhibitors such as ritonavir), ligands (e.g., biotin), nucleic acids (e.g., RNA), oligonucleotides (e.g., antisense oligonucleotides), peptides (e.g., oxytocin), phospholipids (e.g., phosphatidylcholine), prostaglandins (e.g., prostaglandin E2), proteins (e.g., albumin), toxins (e.g., botulinum toxin), vitamins (e.g., vitamin D) and any combination thereof.

Exemplary therapeutically active agents suitable for inclusion in a liposome (e.g., as a therapeutically active agent or as a moiety capable of generating the therapeutically active agent) include, without limitation, amphotericin B, cisplatin, cytarabine, daunorubicin, doxorubicin, estradiol, influenza virosome, morphine, surfactant protein B, surfactant protein C, verteporfin and vincristine.

As used herein, the phrase “labeling agent” refers to a detectable moiety or a probe and includes, for example, chromophores, fluorescent compounds, phosphorescent compounds, heavy metal clusters, and radioactive labeling compounds, as well as any other known detectable moieties.

Examples of a labeling moiety or agent include moieties and compounds which are chromophoric (e.g., absorb visible light), fluorescent, phosphorescent, and/or radioactive. Many such compounds and moieties (and techniques for preparing such moieties) will be known to a skilled person.

A targeting moiety in a liposome according to any of the respective embodiments described herein may optionally be a targeting moiety according to any of the respective embodiments described herein. A targeting moiety in a liposome may be comprised by a co-polymeric compound according to some embodiments of the invention (according to any of the respective embodiments described herein), the liposome comprising the co-polymeric compound. Alternatively or additionally, a targeting moiety in a liposome may optionally be comprised by another compound in the liposome, optionally a bilayer-forming lipid (according to any of the respective embodiments described herein) conjugated to a targeting moiety according to any of the respective embodiments described herein.

In some of any of the embodiments described herein, the liposome may further comprise an additional agent that is bound to or associated with the lipid bilayer. Such agents can be, for example, lipidic agents, including, but not limited to, fatty acids, fatty amines, fatty acyls, fatty alcohols, monoglycerides, diglycerides, triglycerides, glycerophospholipids, sphingolipids, and sterols (e.g., cholesterol).

When a liposome as described herein comprises a therapeutically active agent, bound to, or associated with, a surface of the liposome and/or within a lipid bilayer and/or core of the liposome (e.g., within the liposome bilayer and/or enveloped by the liposome bilayer), the liposome can be used, for example, in drug delivery applications.

According to an aspect of some embodiments of the invention, there is provided a liposome comprising at least one bilayer-forming lipid; a co-polymeric compound; optionally a sterol; and a therapeutically active agent, as described herein in any of the respective embodiments and any combination thereof, which is for use in delivering the therapeutically active agent to a subject in need thereof. The liposome can be used per se or be formulated in a pharmaceutical composition as described herein.

According to an aspect of some embodiments of the present invention, there is provided a method of delivering a therapeutically active agent to a subject in need thereof, the method comprising administering to the subject a liposome comprising at least one bilayer-forming lipid; a co-polymeric compound; optionally a sterol; and a therapeutically active agent, as described herein in any of the respective embodiments and any combination thereof, or a composition comprising the liposome, as described herein, thereby delivering the therapeutically active agent.

According to an aspect of some embodiments of the present invention, there is provided a use of a liposome comprising at least one bilayer-forming lipid; a co-polymeric compound; optionally a sterol; and a therapeutically active agent, as described herein in any of the respective embodiments and any combination thereof, in the manufacture of a medicament for delivering the therapeutically active agent to a subject in need thereof.

As used herein, “delivery” or “delivering” of a therapeutically active agent or drug (which terms are used herein interchangeably) refers to administration of the therapeutically active agent to a subject while controlling duration and/or proportion of the agent at a desired bodily site, depending on a subject's condition (e.g., a bodily site at which the agent desirably exerts a therapeutic effect). Thus, the terms “delivery” and “delivering” (and grammatical variations thereof) encompass targeting of a therapeutically active agent to a specific bodily site, such that a higher proportion of the agent reaches the bodily site (e.g., using a suitable targeting moiety); and/or control over duration of a presence of such an agent in the body (e.g., in the blood)—for example, by sustained release—which may be associated with a duration of such an agent at a desired bodily site (even if in the absence of specific targeting to the bodily site).

As used herein, the phrase “bodily site” includes any organ, tissue, membrane, cavity, blood vessel, tract, biological surface or muscle, which contacting therewith (e.g., delivering thereto or applying thereon) the liposome or the therapeutically active agent disclosed herein is beneficial. Exemplary bodily sites include, but are not limited to, the skin, a dermal layer, the scalp, an eye, an ear, a mouth, a throat, a stomach, a small intestines tissue, a large intestines tissue, a kidney, a pancreas, a liver, the digestive system, the respiratory tract, a bone marrow tissue, a mucosal membrane, a nasal membrane, the blood system, a blood vessel, a muscle, a pulmonary cavity, an artery, a vein, a capillary, a heart, a heart cavity, a male or female reproductive organ and any visceral organ or cavity. Any organ or tissue onto which microorganism can exist in contemplated.

As used herein, “sustained release” refers to a formulation of an agent which provides a gradual and/or delayed (“sustained”) release of the agent (e.g., from a reservoir such a liposome according to any of the respective embodiments described herein), which results in the agent being present in a bodily site (e.g., in the blood upon systemic administration, or in a bodily site to which the agent is locally administered) for a longer duration and/or at a later time (relative to administration) than if the agent is administered per se (via the same administration route).

In some embodiments, the sustained release is characterized by a concentration of therapeutically active agent (e.g., in the blood upon systemic administration, or in a bodily site to which the agent is locally administered) which is at least half of the maximal concentration (Cmax) for a time period which is at least 50% more than a corresponding time period (i.e., during which a concentration of an agent is at least half of the maximal concentration) an agent upon administration of the therapeutically effective agent per se (e.g., as defined herein) in an amount which results in the same maximal concentration. In some such embodiments, the time period (for sustained release) is at least 100% more than (i.e., twice) a corresponding time period (for the agent per se). In some embodiments, the time period (for sustained release) is at least 200% more than (i.e., 3-fold) a corresponding time period (for the agent per se). In some embodiments, the time period (for sustained release) is at least 400% more than (i.e., 5-fold) a corresponding time period (for the agent per se).

Sustained release (according to any of the respective embodiments described herein), may allow, for example, for a regimen characterized by less frequent administration and/or by greater therapeutic efficacy of any given administration. The skilled person will be readily capable of determining a suitable frequency of administration for a given therapeutically active agent based on the duration of the sustained release (e.g., a time period during which the concentration of the agent is at least half of the maximal concentration, according to any of the respective embodiments described herein, and/or at least a minimal effective concentration), and the ratio between a desirable maximal concentration and a minimal effective concentration for the given agent (e.g., a “therapeutic window” of the agent).

### Additional Definitions:

Herein, the term “hydrocarbon” describes an organic moiety that includes, as its basic skeleton, a chain of carbon atoms, substituted mainly by hydrogen atoms. The hydrocarbon can be saturated or non-saturated, be comprised of aliphatic, alicyclic or aromatic moieties, and can optionally be substituted by one or more substituents (other than hydrogen). A substituted hydrocarbon may have one or more substituents, whereby each substituent group can independently be, for example, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, oxo, cyano, nitro, azo, azide, sulfonamide, carboxy, thiocarbamate, urea, thiourea, carbamate, amide, and hydrazine. The hydrocarbon can be an end group or a linking group, as these terms are defined herein. The hydrocarbon moiety is optionally interrupted by one or more heteroatoms, including, without limitation, one or more oxygen, nitrogen and/or sulfur atoms. In some embodiments of any of the embodiments described herein relating to a hydrocarbon, the hydrocarbon is not interrupted by any heteroatoms.

Preferably, the hydrocarbon moiety has 1 to 20 carbon atoms. Whenever a numerical range; e.g., “1 to 20”, is stated herein, it implies that the group, in this case the alkyl group, may contain 1 carbon atom, 2 carbon atoms, 3 carbon atoms, etc., up to and including 20 carbon atoms.

Herein, the term “alkyl” describes a saturated aliphatic hydrocarbon end group, as defined herein, including straight chain and branched chain groups. Preferably, the alkyl group has 1 to 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted. Substituted alkyl may have one or more substituents, whereby each substituent group can independently be, for example, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine.

The term “alkylene” describes a saturated aliphatic hydrocarbon linking group, as this term is defined herein, which differs from an alkyl group, as defined herein, only in that alkylene is a linking group rather than an end group.

Herein, the term “alkenyl” describes an unsaturated aliphatic hydrocarbon end group which comprises at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted. Substituted alkenyl may have one or more substituents, whereby each substituent group can independently be, for example, cycloalkyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine.

Herein, the term “alkynyl” describes an unsaturated aliphatic hydrocarbon end group which comprises at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Substituted alkynyl may have one or more substituents, whereby each substituent group can independently be, for example, cycloalkyl, alkenyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or non-substituted. Substituted cycloalkyl may have one or more substituents, whereby each substituent group can independently be, for example, alkyl, alkenyl, alkynyl, cycloalkyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine. The cycloalkyl group can be an end group, as this phrase is defined herein, wherein it is attached to a single adjacent atom, or a linking group, as this phrase is defined herein, connecting two or more moieties.

The term “aryl” describes an all-carbon monocyclic or fused-ring polycyclic (i.e., rings which share adjacent pairs of carbon atoms) end group (as this term is defined herein) having a completely conjugated pi-electron system. The aryl group may be substituted or non-substituted. Substituted aryl may have one or more substituents, whereby each substituent group can independently be, for example, alkyl, alkenyl, alkynyl, cycloalkyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine. Phenyl and naphthyl are representative aryl end groups.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. Substituted heteroaryl may have one or more substituents, whereby each substituent group can independently be, for example, alkyl, alkenyl, alkynyl, cycloalkyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine. The heteroaryl group can be an end group, as this phrase is defined herein, where it is attached to a single adjacent atom, or a linking group, as this phrase is defined herein, connecting two or more moieties. Representative examples are pyridine, pyrrole, oxazole, indole, purine and the like.

The term “arylene” describes a monocyclic or fused-ring polycyclic linking group, as this term is defined herein, and encompasses linking groups which differ from an aryl or heteroaryl group, as these groups are defined herein, only in that arylene is a linking group rather than an end group.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. Substituted heteroalicyclic may have one or more substituents, whereby each substituent group can independently be, for example, alkyl, alkenyl, alkynyl, cycloalkyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine. The heteroalicyclic group can be an end group, as this phrase is defined herein, where it is attached to a single adjacent atom, or a linking group, as this phrase is defined herein, connecting two or more moieties. Representative examples are piperidine, piperazine, tetrahydrofuran, tetrahydropyran, morpholine and the like.

As used herein, the terms “amine” and “amino” describe both a —NRxRy end group and a —NRx- linking group, wherein Rx and Ry are each independently hydrogen, alkyl, cycloalkyl, aryl, heteroaryl or heteroalicyclic, as these terms are defined herein. When Rx or Ry is heteroaryl or heteroalicyclic, the amine nitrogen atom is bound to a carbon atom of the heteroaryl or heteroalicyclic ring. A carbon atom attached to the nitrogen atom of an amine is not substituted by ═O or ═S, and in some embodiments, is not substituted by any heteroatom.

The amine group can therefore be a primary amine, where both Rx and Ry are hydrogen, a secondary amine, where Rx is hydrogen and Ry is alkyl, cycloalkyl, aryl, heteroaryl or heteroalicyclic, or a tertiary amine, where each of Rx and Ry is independently alkyl, cycloalkyl, aryl, heteroaryl or heteroalicyclic.

The terms “hydroxy” and “hydroxyl” describe a —OH group.

The term “alkoxy” describes both an —O-alkyl and an —O-cycloalkyl end group, or —O-alkylene or —O-cycloalkyl linking group, as defined herein.

The term “aryloxy” describes both an —O-aryl and an —O-heteroaryl end group, or an —O-arylene-linking group, as defined herein.

The term “thiohydroxy” describes a —SH group.

The term “thioalkoxy” describes both an —S-alkyl and an —S-cycloalkyl end group, or —S-alkylene or —S-cycloalkyl linking group, as defined herein.

The term “thioaryloxy” describes both an —S-aryl and an —S-heteroaryl end group, or an —S-arylene-linking group, as defined herein.

The terms “cyano” and “nitrile” describe a —C≡N group.

The term “nitro” describes an —NO₂ group.

The term “oxo” describes a ═O group.

The term “azide” describes an —N═N+=N group.

The term “azo” describes an —N═N-Rx end group or —N═N=linking group, with Rx as defined herein.

The terms “halide” and “halo” refer to fluorine, chlorine, bromine or iodine.

The term “phosphate” refers to a —O—P(═O)(ORx)-OR_{Y} end group, or to a —O—P(═O)(ORx)-O— linking group, where Rx and R_{Y} are as defined herein.

The terms “phosphonyl” and “phosphonate” refer to an —P(═O)(ORx)-OR_{Y} end group, or to a —P(═O)(ORx)-O— linking group, where Rx and R_{Y} are as defined herein. The term “phosphinyl” refers to a —PRxR_{Y} group, where Rx and R_{Y} are as defined hereinabove.

The term “sulfoxide” or “sulfinyl” describes a —S(═O)—Rx end group or —S(═O)— linking group, where Rx is as defined herein.

The terms “sulfonate” and “sulfonyl” describe a —S(═O)₂—Rx end group or —S(═O)₂— linking group, where Rx is as defined herein.

The terms “sulfonamide” and “sulfonamido”, as used herein, encompass both S-sulfonamide and N-sulfonamide end groups, and a —S(═O)₂—NRx- linking group.

The term “S-sulfonamide” describes a —S(═O)₂—NRxR_{Y} end group, with Rx and R_{Y} as defined herein.

The term “N-sulfonamide” describes an RxS(═O)₂—NR_{Y}— end group, where Rx and R_{Y} are as defined herein.

The term “carbonyl” as used herein, describes a —C(═O)—Rx end group or —C(═O)— linking group, with Rx as defined herein.

The term “acyl” as used herein, describes a —C(═O)—Rx end group, with Rx as defined herein.

The term “thiocarbonyl” as used herein, describes a —C(═S)—Rx end group or —C(═S)— linking group, with Rx as defined herein.

The terms “carboxy” and “carboxyl”, as used herein, encompasses both C-carboxy and O-carboxy end groups, and a —C(═O)—O— linking group.

The term “C-carboxy” describes a —C(═O)—ORx end group, where Rx is as defined herein.

The term “O-carboxy” describes a —OC(═O)—Rx end group, where Rx is as defined herein.

The term “urea” describes a —NRxC(═O)—NRyRw end group or —NRxC(═O)—NRy- linking group, where Rx and Ry are as defined herein and Rw is as defined herein for Rx and Ry.

The term “thiourea” describes a —NRx-C(═S)—NRyRw end group or a —NRx-C(═S)—NRy-linking group, with Rx, Ry and Ry as defined herein.

The terms “amide” and “amido”, as used herein, encompasses both C-amide and N-amide end groups, and a —C(═O)—NRx- linking group.

The term “C-amide” describes a —C(═O)—NRxRy end group, where Rx and Ry are as defined herein.

The term “N-amide” describes a RxC(═O)—NRy- end group, where Rx and Ry are as defined herein.

The term “carbamyl” or “carbamate”, as used herein, encompasses N-carbamate and O-carbamate end groups, and a —OC(═O)—NRx- linking group.

The term “N-carbamate” describes a RyOC(═O)—NRx- end group, with Rx and Ry as defined herein.

The term “O-carbamate” describes an —OC(═O)—NRxRy end group, with Rx and Ry as defined herein.

The term “thiocarbamyl” or “thiocarbamate”, as used herein, encompasses O-thiocarbamate, S-thiocarbamate and N-thiocarbamate end groups, and a —OC(═S)—NRx- or —SC(═O)—NRx- linking group.

The terms “O-thiocarbamate” and “O-thiocarbamyl” describe a —OC(═S)—NRxRy end group, with Rx and Ry as defined herein.

The terms “S-thiocarbamate” and “S-thiocarbamyl” describe a —SC(═O)—NRxRy end group, with Rx and Ry as defined herein.

The terms “N-thiocarbamate” and “N-thiocarbamyl” describe a RyOC(═S)NRx- or RySC(═O)NRx- end group, with Rx and Ry as defined herein.

The term “guanidine” describes a —RxNC(═N)—NRyRw end group or —RxNC(═N)—NRy-linking group, where Rx, Ry and Rw are as defined herein.

The term “hydrazine”, as used herein, describes a —NRx-NRyRw end group or —NRx-NRy-linking group, with Rx, Ry, and Rw as defined herein.

As used herein the term “about” refers to +10% or +5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

Materials: 2-Methacryloyloxyethyl phosphorylcholine (MPC, 97%), N-isopropylacrylamide (NIPAM, ≥99%), ammonium persulfate (APS, 98%), N,N,N′,N′-tetramethylethylenediamine (TMEDA, 99%), 2-hydroxyethyl methacrylate (HEMA, 99%), ethylene glycol dimethacrylate (EGDMA, 98%), deuterium oxide (99.9 atom % D), Transwell® cell culture inserts (12 mm inserts, 0.4 μm pore size), XTT cell proliferation assay, Arabinose were obtained from Sigma-Aldrich™ (Israel). Ampicillin was obtained from Tivan Biotech (Israel). RPMI medium (Gibco), fetal bovine serum (FBS; Gibco), penicillin-streptomycin, Vybrant DiD cell-labeling solution, pBAD Kit (V43001) were obtained from Thermo Fisher Scientific Inc. (Israel). Water used was purified using a Barnstead™ Nanopure™ system (Thermo Fisher Scientific, Waltham, MA, USA) to MΩ·cm (at 25° C.) and total organic content <1 ppb. N-propylacrylamide (nPAM, >98%) was obtained from Tokyo Chemical Industry Co., Ltd.

### Hydrogel Preparation:

PMN-free pHEMA hydrogels were prepared as follows: HEMA (3 mL), EGDMA (0.1 mL or 0.3 mL), APS aqueous solution (0.2 mL, 24 mg APS), and purified water (2 mL) were added together and vigorously stirred for 15 minutes at room temperature. The molar ratio of the EGDMA crosslinker to the HEMA monomer was kept at 2%. To accelerate the reaction, TMEDA (50 μL) was added to the mixture, which was then stirred for 20 seconds and poured into a 6 centimeter (cm) diameter petri dish. The mixture was allowed to gel at room temperature for 4 hours, followed by immersion in an excess of purified water for 3 days to remove unreacted materials. This resulted in very slight swelling of the pHEMA hydrogels, whose final water content was measured at 38.3±0.3% of the hydrogel weight (determined by weight-changes upon freeze-drying). Obtained hydrogels were cut into circular disks of 20 mm diameter and thickness of about 2 mm, and the surface exposed to air during the gelation was used for measurements.

PMN-incorporating hydrogels were prepared similarly, while replacing the 2 mL purified water by 2 mL of a respective PMN-containing solution at a concentration of 25 mg/mL, to provide a 1% PMN-incorporating pHEMA hydrogel (1% by weight).

PMP-incorporating hydrogels (1% by weight) were prepared in a similar manner, with the exception that the PMN solution (2 mL) was replaced by the respective PMP solution (2 mL). An exemplary preparation process is as follows:

HEMA (3 mL), EGDMA (0.1 mL or 0.3 mL), APS aqueous solution (0.2 mL, 24 mg APS), and PMP copolymer solution (25 mg/mL, 2 mL) were added together and vigorously stirred for 15 minutes at room temperature. TMEDA (50 μL) was added to the mixture, which was then stirred for 20 seconds and poured into a 6 cm diameter petri dish. The mixture was allowed to gel at room temperature for 4 hours, and unreacted materials were removed by immersion in an excess of purified water for 3 days, to provide a PMP-incorporating hydrogel.

PMN-rhodamine conjugate-incorporating hydrogels were similarly prepared, while replacing the 2 mL purified water by 2 mL aqueous solution of PMN and 0.5 weight % of acryloxyethyl thiocarbamoyl rhodamine B.

DMPC-incorporating hydrogels were prepared by the following procedures:

HEMA (3 mL, 24.6 mmol), EGDMA (generally 0.095 mL, 0.5 mmol), APS aqueous solution (0.2 mL containing 0.105 mmol APS), and 45 mM DMPC aqueous suspension (2 mL) were added together and vigorously stirred for 0.5 hour. The molar ratio of the EGDMA cross-linker to the HEMA monomer was thus generally 2%. TMEDA (50 μL) was added to the mixture, stirring took place for 20 seconds and the mixture was poured into a 6-cm diameter petri dish. The mixture was allowed to gel at room temperature for 4 hours, followed by immersion in a large excess of purified water for 3 days to remove unreacted materials.

pNIPAM-incorporating, pMPC-incorporating and pNIPAM+pMPC-incorporating hydrogels were prepared by the following procedures:

pNIPAM-incorporating hydrogels: HEMA (3 mL=24.6 mmol), EGDMA (generally 0.095 mL, 0.5 mmol), APS aqueous solution (0.2 mL containing 0.105 mmol APS), and 25 mg/mL pNIPAM aqueous solution (2 mL) were added together and vigorously stirred for 0.5 hour. The molar ratio of the EGDMA cross-linker to the HEMA monomer was thus generally 2%. TMEDA (50 μL) was added to the mixture, stirring took place for 20 seconds and the mixture was poured into a 6-cm diameter petri dish. The mixture was allowed to gel at room temperature for 4 hours, followed by immersion in a large excess of purified water for 3 days to remove unreacted materials.

pMPC-incorporating hydrogel: HEMA (3 mL, 24.6 mmol), EGDMA (generally 0.095 mL, 0.5 mmol), APS aqueous solution (0.2 mL containing 0.105 mmol APS), and 12.5 mg/mL pMPC aqueous solution (2 mL) were added together and vigorously stirred for 0.5 hour. The molar ratio of the EGDMA cross-linker to the HEMA monomer was thus generally 2%. TMEDA (50 μL) was added to the mixture, stirring took place for 20 seconds and the mixture was poured into a 6-cm diameter petri dish. The mixture was allowed to gel at room temperature for 4 hours, followed by immersion in a large excess of purified water for 3 days to remove unreacted materials.

pNIPAM+pMPC-incorporating hydrogels: HEMA (3 mL, 24.6 mmol), EGDMA (generally 0.095 mL, 0.5 mmol), APS aqueous solution (0.2 mL containing 0.105 mmol APS), and 25 mg/mL pNIPAM+12.5 mg/mL pMPC aqueous solution (2 mL) were added together and vigorously stirred for 0.5 hour. The molar ratio of the EGDMA cross-linker to the HEMA monomer was thus generally 2%. TMEDA (50 μL) was added to the mixture, stirring took place for 20 seconds and the mixture was poured into a 6-cm diameter petri dish. The mixture was allowed to gel at room temperature for 4 hours, followed by immersion in a large excess of purified water for 3 days to remove unreacted materials.

HEMA-co-HEAA hydrogel: HEMA (1.5 mL, 12.3 mmol), HEAA (1.5 mL, 14.5 mL), EGDMA (generally 0.095 mL, 0.5 mmol), APS aqueous solution (0.2 mL containing 0.105 mmol APS), and 2 mL pure water were added together and vigorously stirred for 0.5 hour. The molar ratio of the EGDMA cross-linker to the HEMA monomer was thus generally 2%. TMEDA (50 μL) was added to the mixture, stirring took place for 20 seconds and the mixture was poured into a 6-cm diameter petri dish. The mixture was allowed to gel at room temperature for 4 hours, followed by immersion in a large excess of purified water for 3 days to remove unreacted materials.

HEMA-co-DMAA hydrogel: HEMA (1.5 mL, 12.3 mmol), DMAA (1.5 mL, 16.1 mL), EGDMA (generally 0.095 mL, 0.5 mmol), APS aqueous solution (0.2 mL containing 0.105 mmol APS), and 2 mL pure water were added together and vigorously stirred for 0.5 hour. The molar ratio of the EGDMA cross-linker to the HEMA monomer was thus generally 2%. TMEDA (50 μL) was added to the mixture, stirring took place for 20 seconds and the mixture was poured into a 6-cm diameter petri dish. The mixture was allowed to gel at room temperature for 4 hours, followed by immersion in a large excess of purified water for 3 days to remove unreacted materials.

PEGMA hydrogel: PEGMA (3 mL, 8.3 mmol), EGDMA (generally 0.095 mL, 0.5 mmol), APS aqueous solution (0.2 mL containing 0.105 mmol APS), and 2 mL pure water were added together and vigorously stirred for 0.5 hour. The molar ratio of the EGDMA cross-linker to the HEMA monomer was thus generally 2%. TMEDA (50 μL) was added to the mixture, stirring took place for 20 seconds and the mixture was poured into a 6-cm diameter petri dish. The mixture was allowed to gel at room temperature for 4 hours, followed by immersion in a large excess of purified water for 3 days to remove unreacted materials.

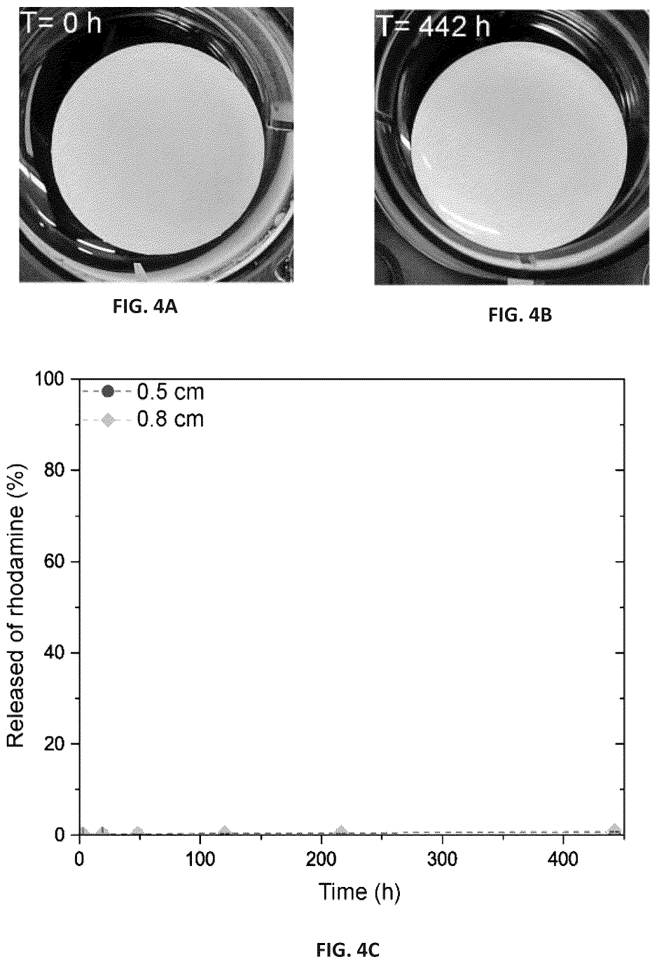

The photographs presented in FIGS. 4A-B and FIGS. 8A-C were acquired using a 8 MP Sony IMX586 1/2.0″ sensor, f/1.75-aperture lens with 26 mm-equivalent focal length, equipped on a OnePlus Nord phone (OnePlus, China).

¹H NMR analyses were performed on a Bruker Avance-III 300 Hz NMR.

Cryo-SEM freeze fracture imaging: Hydrogel samples (20 mm disks) were sectioned to slices of thickness 100 μm using a vibratome (Electron Microscopy Sciences, USA). The slices were sandwiched between an aluminum disc with a depression of depth 150 μm and a flat disc (M. Wohlwend GmbH, Switzerland) and were then cryo-immobilized by HPM010 High Pressure Freezing Machine (BalTech, Liechtenstein). The frozen sample was removed from the disc under liquid nitrogen, mounted perpendicularly in a holder, transferred to a BAF060 Freeze-fracturing device (Leica Microsystems, Austria) using a Leica EM VCT100 cryo transfer shuttle (Leica Microsystems, Austria) and was fractured perpendicularly to the plane of the frozen slice at −120° C. under a pressure of about 5.10-7 mbar. The cold fractured surface was sometimes “etched” by increasing the temperature to about-105° C. for several minutes to let some frozen water sublime. The fractured sample was then transferred to an Ultra 55 cryo-scanning electron microscope (Zeiss, Germany) and observed using an InLens Secondary electrons detector at an acceleration voltage ranging between 1-2.5 kV.

Rheometry: Viscoelastic measurements were performed using a strain/stress-controlled rheometer (Thermo-Haake, Mars III, Karlsruhe, Germany). Samples were prepared in the form of disks with a diameter of 20 mm and thickness of about 2 mm. Samples were tested using a parallel plate geometry. The samples were placed between the rheometer plates and a slight compression of about 1.0 N was applied. Amplitude sweep tests were performed in constant strain mode for strains in the range of 10⁻⁴ to 0.5, the oscillation frequency being 1 Hz. Frequency sweep tests were performed in stress control mode, with stress being in the range of linear viscoelasticity, as determined from the amplitude sweep studies. The frequency range was between 0.05 and 30-100 Hz.

Friction measurments: Friction measurements of several independent experiments (different hydrogel samples with at least 3 different contact points) for each set of conditions, were carried out using a CETR UMT tribometer (Bruker, MA, USA) with normal and friction force sensors under PBS at 37° C. Measurements were done on hydrogel samples (20 mm disks) and carried out at different sliding velocities (vs) and loads (Fₙ) and under high-purity water (unless stated differently). Loads used were in the range 1.5-30 N. Fₛ was determined from its value in the plateau region of the friction traces (friction coefficients evaluated at each load as μ=Fₛ/Fₙ).

Dynamic light scattering (DLS) and Dialysis: pHEMA (20 kDa, without PMN) was dissolved in ethanol/DMF (v/v=1/1), and pippeted (about 5 μL] into water with final pHEMA concentration of 0.2 mg/mL. The mixture was then dialysed against water for 2 days. Size distribution and zeta (z) potential of the samples were characterized using a Zetasizer Nano-ZS instrument (Malvern Instruments Ltd., Malvern, WR, U.K.) equipped with a red laser (633 nm) and a scattering angle of 173°.

Confocal fluorescence microscopy imaging: To prepare PMN-based hydrogels for confocal fluorescence microscopy imaging, PMN-rhodamine conjugated based hydrogels were prepared as described hereinabove. Confocal pictures were acquired using confocal laser scanning microscopy LSM700 (Zeiss, Germany). All images were acquired using a 40× oil immersion objective (NA 1.4 Zeiss, Germany) and recorded in brightfield mode and in confocal mode using a 540 nm excitation laser channel, with a 0.3 μm optical slice step for z scanning. Picture analysis was performed using ImageJ software v1.52i (NIH, USA).

Commercialized catheter coating: Actreen® Hi-Lite Cath Nelaton (glycerol-coated catheter for male intermittent self-catheterization) was washed with excess water to remove the existing lubricant, to thereby provide an uncoated commercial catheter. The cleaned catheter was plasma (O₂/Ar=1/1) treated for 5 minutes, and then immersed into 3% 3-(trimethoxysilyl) propyl methacrylate (toluene solution) for 12 hours. The catheter was washed with toluene, and dried in the air. Hydrogel coating (w/o PMN) on the catheter was performed by pulling out the catheter from the polymerization solution after 1 minute, and the polymerization was proceeding for 12 hours under N₂ atmosphere.

Cell culture: Vero E6 cells were cultured in RPMI medium, supplemented with 1% Pen/Strep and 10% fetal bovine serum (FBS), and were maintained at 37° C. in a humidified atmosphere of 95% air and 5% CO₂.

### Cytotoxicity Studies:

Cell culture: Prior to cytotoxicity experiment, cells were cultured in 12-well plates at 8·10⁴ cells per mL and allowed to attach for 24 hours.

Cytotoxicity evaluation: The cytotoxicity of hydrogel containing 3 mg/mL PMN was conducted in 12-well plates using Transwell® cell culture inserts. Hydrogel (0.6 mm disk) was placed in a center of Transwell® and pre-incubated in a cell culture media for 1 hour at room temperature in order to exchange water within the hydrogel. The inserts with hydrogels were then placed into wells containing the cultured cells and incubated for 48 hours at 37° C. (95% air and 5% CO₂).

Cell viability: cell viability was determined by the production of the yellow formazan product upon cleavage of XTT by mitochondrial dehydrogenases in viable Vero cells. After 48 hours incubation with hydrogels, the cells were incubated with 500 μL of XTT solution for 4 hours at 37° C. (95% air and 5% CO₂). Absorbance values were later measured with ClarioStar microplate reader (BMG LABTECH GmbH, Germany) at a wavelength of 450 nm. Background absorbance was measured at 620 nm and subtracted from the 450 nm measurement. The absorbance of a solution in RPMI-cell-free media with XTT was subtracted from all samples. The potential toxic effect of the different liposomal formulations tested was expressed as a viability percentage calculated using the following formula:

% Viability=(ODtest/ODc)·100%

Where ODtest was the optical density of those wells with hydrogels, and ODc was the optical density of cells in RPMI media only (no hydrogel).

In-vitro PMN release studies: PMN (Rhodamine-conjugate) hydrogels were placed in 2 mL PBS (pH 7.2, osmol=290 mOsmo/kg) in 12-well plates and incubated at 37° C. (95% air and 5% CO₂) to mimic physiological conditions. At specific time points, 2 mL of PBS were taken and replaced with fresh buffer. The retrieved conditioned PBS was evaporated using a lyophilizer followed by resuspension in 0.2 mL PBS. The concentration of PMN-rhodamine-conjugate in PBS was determined with a ClarioStar microplate reader (BMG LABTECH GmbH, Germany) at a wavelength of 566 nm. PMN-rhodamine-conjugate stock solution used for hydrogel preparation was diluted to specific concentrations and used as a standard calibration curve. The percentage of the cumulative amount of released PMN was calculated from a standard calibration curve.

Fluorescence-based monitoring of gel-to-stainless steel (SS) and gel-to-polyethylene (PE) transfer of PMN-rhodamine conjugate during sliding: PMN-rhodamine conjugate-incorporating hydrogels were prepared as described hereinabove. PMN transfer via SS-gel (stainless steel, SS) or PE-gel (polyethylene, PE) sliding and imaging of transferred material. The spherical head (SS or PE) was slid past hydrogels incorporating PMN-rhodamine-conjugate or DMPC-MLV stained with 1% DiI, at a sliding velocity 1 mm/second for 5 minutes. It was then placed in a petri dish and the area of absorbed polymer or lipids transferred during the sliding was imaged by excitation of rhodamine and DiI dye at 532 nm using a Typhoon FLA 9500 scanner (GE Healthcare Bio-Sciences AB, Sweden), with photomultiplier tube set up at 500′ gain and pixel size 50 μm. Pictures were analyzed using ImageJ software (NIH, USA).

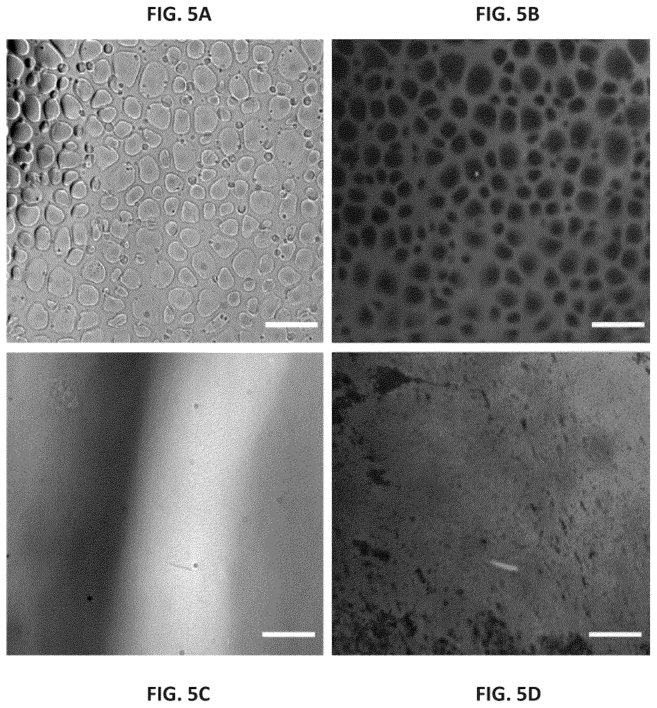

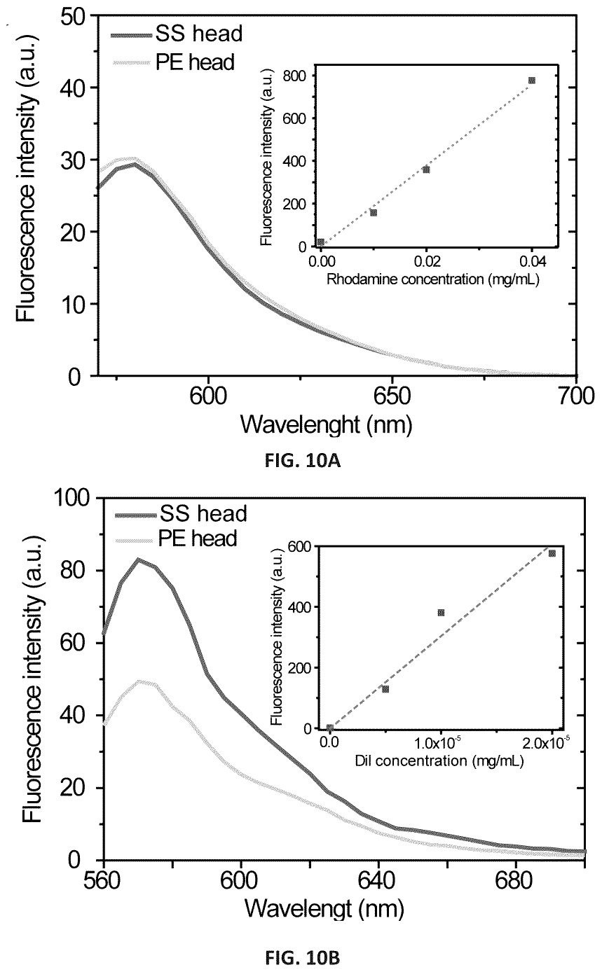

Subsequently, the spherical head (PE or SS) head was washed with water (PMN) or ethanol (DMPC), and evaporated overnight using a nitrogen stream followed by lyophilization. 400 μL of water (PMN) or chloroform (DMPC) was used to re-suspend polymer or lipids, and the solution was placed in a quartz silica cuvette with a 1 mm path length. Acquisition of rhodamine and DiI emission spectra was performed with an Agilent Cary Eclipse Fluorescence Spectrophotometer (Varian Instruments, Walnut Creek, CA) at room temperature (FIGS. 5A-D). The excitation wavelength was set at 500 nm with a bandpass of 20 nm, and the emission was also recorded with bandpass of 20 nm. Spectrum acquisition was repeated over a minimum of 3 separate samples. Calibration curves were prepared by measuring the maximal (Inserts of FIGS. 10A and 10B) fluorescence intensity of known amounts of pure PMN-Rhodamine-water and DMPC-DiI-chloroform solution by the same method and parameters to ensure the same experimental conditions.

### Anti-Biofouling Measurements:

Vero E6 cells adhesion to hydrogels: Vero E6 cell suspension (1.5 mL of 5.2.105) was seeded onto 6 mm hydrogel discs (neat and PMN) in 24 well plates and allowed to attach for 24 hours. The cells were then stained with Vybrant DiD by adding 5 μL of stock solution and incubated for 20 minutes at 37° C. After 20 minutes hydrogels were washed carefully with PBS and placed in a fresh cell medium. Hydrogels were placed on the slide and observed with confocal laser scanning microscopy LSM700 (Zeiss, Germany). All images were acquired using a 60× oil immersion objective and recorded in brightfield mode and in confocal mode using a 635 nm excitation laser channel. The anti-biofouling property of hydrogel was further determined by using Typhoon FLA 9500 scanner (GE Healthcare Bio-Sciences AB, Sweden) with 635 nm laser wavelength, photomultiplier tube set up at 250′ gain and pixel size 25 μm. Pictures were analyzed using ImageJ software (NIH, USA). For comparative analysis, all parameters during image acquisition were kept constant throughout each experiment. Additionally, fluorescence signal on hydrogel surfaces was determined with a ClarioStar microplate reader (BMG LABTECH GmbH, Germany) in 96-well plate at a wavelength of 630 nm with top reading and spiral scan mode (scan diameter: 6 mm, No. of flashes per well: 106).

Bacteria adhesion to hydrogels: GFP-expressing E. coli culture was developed by plasmid transfection. The sf-gfp-his plasmid was cloned into a pBAD24. The plasmid was then transformed into E. coli MG1655 imp4213. E. coli (MG1655) precultures cultures were inoculated at OD600 0.05 from an overnight culture, and growth was carried out at 37° C. with shaking for 4 hours, in Luria-Bertani broth (LB) supplemented with ampicillin (100 mg/L) and 0.2% arabinose until mid-exponential phase (OD at 600 nm is about 0.5). Then bacteria were diluted in BM2G minimal medium [62 mM potassium phosphate buffer, pH=7, 7 mM (NH₄)₂SO₄, 2 mM MgSO₄, 10 μM FeSO₄, 0.4% (wt/vol) glucose, 1% Monosodium glutamate] to reach OD 0.08. Hydrogel discs (diameter 6 mm) were placed in 24-well plate and 1 mL of bacteria suspension was added and incubated at 37° C. without shaking for 12 hours. Subsequently, hydrogels were washed carefully with PBS and placed in fresh PBS. Hydrogels were placed on the slide and observed with confocal laser scanning microscopy LSM700 (Zeiss, Germany).

All images were acquired using a 60× oil immersion objective and recorded in brightfield mode and in confocal mode using a 473 nm excitation laser channel.

Hydrogels were further visualized using Typhoon FLA 9500 scanner (GE Healthcare Bio-Sciences AB, Sweden) with 635 nm laser wavelength, photomultiplier tube set up at 250′ gain, and pixel size 25 μm.

Pictures were analyzed using ImageJ software (NIH, USA). For comparative analysis, all parameters during image acquisition were kept constant throughout each experiment.

Fluorescence signal on hydrogel surfaces was determined with a CLARIOstar® microplate reader (BMG LABTECH GmbH, Germany) in 96-well plate at a wavelength of 470 nm with top reading and spiral scan mode (scan diameter: 6 mm, No. of flashes per well 106).

### Example 1

### Design, Chemical Synthesis and Structural Characterization

Healthy articular cartilage has excellent lubricating properties, with friction coefficients (μ) reaching about 0.002-0.02 at physiological pressures. Such high-performing lubricating layer in joints is attributed to the surface hydration arising from the interplay between multiple hydrophilic biopolymers (e.g., hyaluronic acid, proteoglycans, lubricin) and phospholipids in the cartilage matrix. Mimicking such molecular structure, hydrogels composed of a hydrophilic polymer network have the potential to replicate the lubricating feature and possibly replace natural cartilages.

The present inventors have previously uncovered that reducing a friction coefficient of a surface can be performed via the hydration lubrication mechanism [Goldberg et al., 2011, supra; Seror et al., 2015, supra; Schmidt, T. A., 2020, supra].

WO 2017/109784 describes lipid-polymer conjugates (LPCs) comprising highly hydrated phosphatidylcholine (PC) lipid headgroups, which, when incorporated in hydrogels, displayed reduction in friction and wear relative to the lipid-free hydrogels over a wide range of conditions. The reduction in friction and wear has been attributed to a lipid-based boundary layer at the hydrogel surface, which is continually reconstructed as it wears, through progressive release of lipids. This effect persists when the hydrogels are dried and then rehydrated. However, the reduction in friction has been observed when the counter surface is a hydrophilic surface (such as glass, hydrogel, or stainless steel) but not with hydrophobic surfaces (e.g., polyethylene), which are implicated in many applications, such as artificial joints and plastic stents.

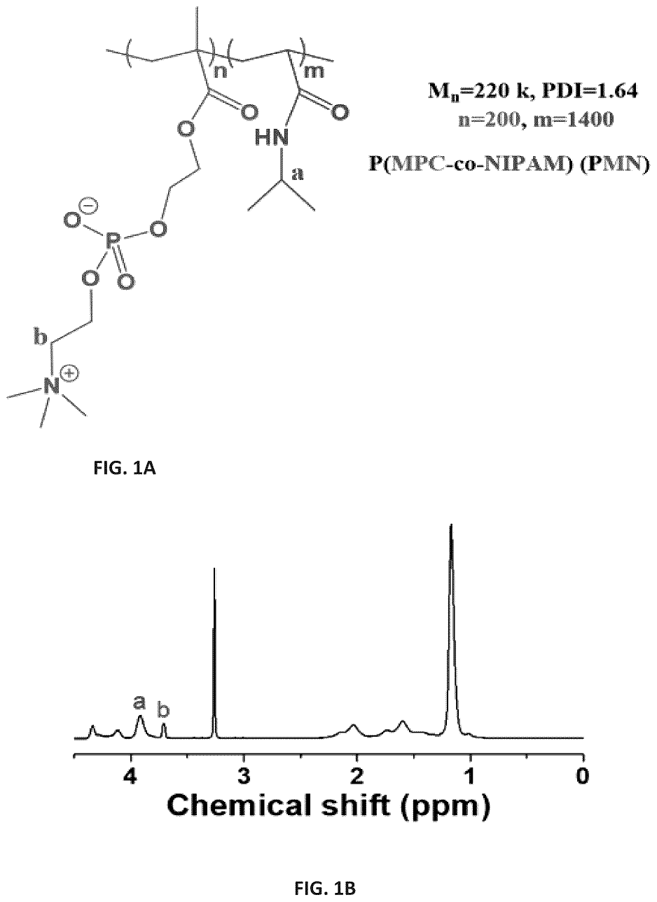

In a search for improved lubrication methodologies, the present inventors have conceived utilizing a polymeric structure featuring positively-charged phosphoryl alkylamine headgroups (e.g., phosphoryl choline), which exhibits a more hydrophobic nature, and have designed, to this effect, as an exemplary co-polymeric compound, poly(2-methacryloyloxyethyl phosphorylcholine-co-N-isopropylacrylamide) random co-polymer (which is also referred to herein throughout as pMPC-co-pNIPAM; p (MPC-co-NIPAM); or simply as PMN) (see, FIG. 1A).

The random copolymer PMN was prepared as follows:

Preparation of a poly(2-methacryloyloxyethyl phosphorylcholine-co-N-isopropylacrylamide) random copolymer (with the content of the 12.5 mol % MPC unit): MPC (3 gram, 10.2 mmol), NIPAM (12 gram, 106 mmol), and APS (320 milligram (mg), 1.4 mmol) were dissolved in 200 mL of pure water. The solution was deoxygenated under nitrogen atmosphere for 30 minutes. After injecting to the solution 44 μL TMEDA, polymerization was carried out at room temperature for 16 hours under a nitrogen atmosphere. The reaction mixture was thereafter dialyzed against pure water for 48 hours and then dehydrated by freeze-drying, to thereby obtain the random copolymer (14.7 grams, 91.9% yield). The chemical structure was verified by ¹H NMR (400 MHz, DMSO), a portion of which is presented in FIG. 1B.

As can be seen from the ¹H NMR spectra on FIG. 1B, the characteristic peak for NIPAM methine protons is at δ=3.9 ppm, while MPC pendant methylene protons show a peak at δ=3.7 ppm. The estimated 7:1 ratio between NIPAM and MPC was determined by comparing the integral intensity signal. The number-average molecular weight (Mn) and the molecular weight distribution (Mw/Mn) as determined by a gel-permeation chromatography (GPC) analysis were 2.2.105 and 1.64, respectively. The number of backbone units of MPC and NIPAM can be calculated to be about 200 and 1400, respectively.

### Example 2

### Lubrication

In order to create a molecularly thin, lipid-based boundary layer that continuously renews, trace lipid concentrations were incorporated into hydrogels.

For this purpose, the exemplary PMN co-polymer was incorporated into biocompatible pHEMA hydrogels by mixing a low concentration (1% wt.) of PMN with the desired HEMA solution, as described in further details in the Materials and Methods section hereinabove.

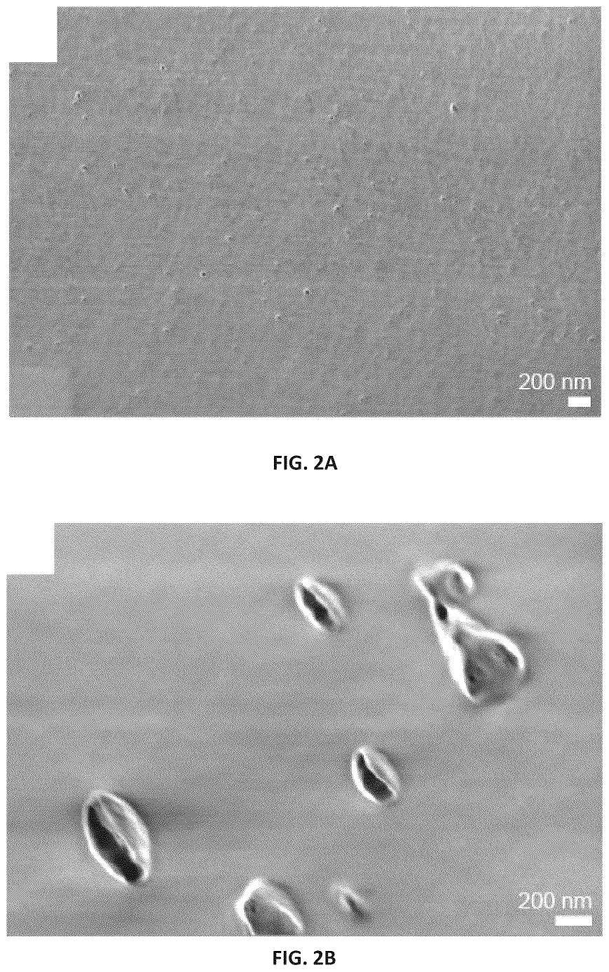

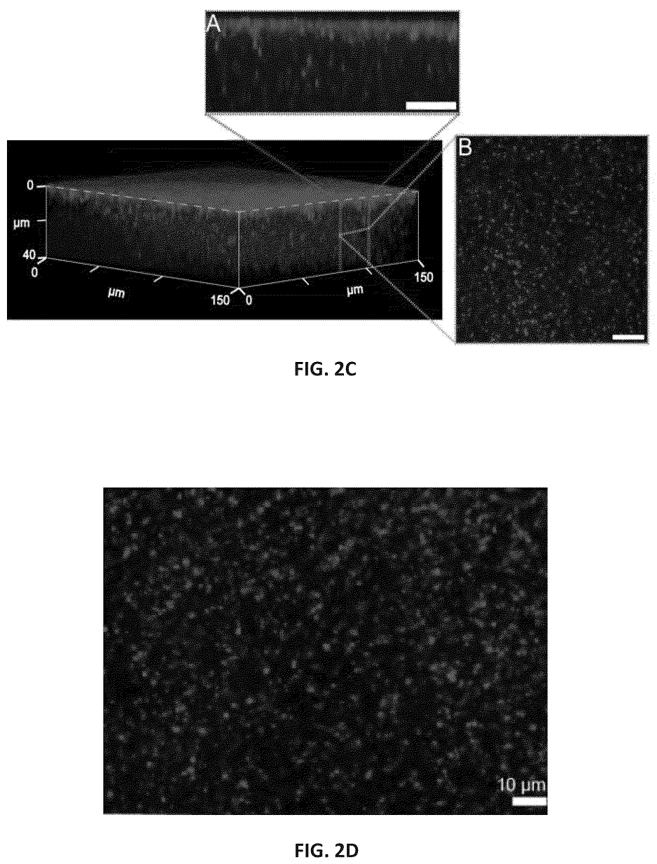

Following polymerization and cross-linking, freeze-fracture cryo-scanning electron microscopy (cryo-SEM) was performed on the resulting pHEMA hydrogels, and the obtained images are presented in FIGS. 2A-B.

As can be seen, the images reveal that PMN-pockets (or, PMN-micro-reservoirs) size ranges from 200 nm up to 900 nm (FIG. 2B) with homogenous distribution throughout the hydrogel bulk, while PMN-free hydrogels show featureless internal structure (FIG. 2A). This observation was further confirmed by confocal microscopy imaging (FIGS. 2C-D).

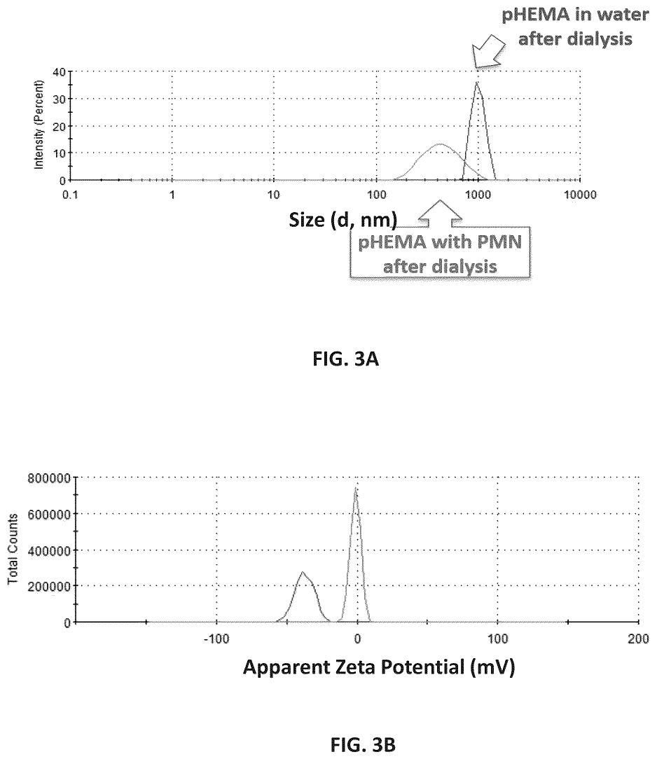

In order to study the interaction between pHEMA and PMN in an aqueous environment, size and zeta potential measurements were performed before and after dialysis (cellulose membrane, molecular weight cut off: 3500) for 2 days, and the results are presented in FIGS. 3A-B. pHEMA was found to be water insoluble as a precipitation was observed after dialysis. The size distribution of pHEMA/PMN dispersion shows one peak without precipitation (FIG. 3A), which could be attributed to the interaction between pHEMA and NIPAM moiety, whereby the hydrated pMPC moieties are exposed.

In-vivo cumulative release of PMN-rhodamine conjugate from hydrogel was performed as described hereinabove, and the obtained data is presented in FIGS. 4A-C. As can be seen, under physiological conditions, PMN pockets within pHEMA hydrogels remained stable for long-term, with less than 1% PMN leakage from the cavities over 18 days.

The incorporation of PMN alters the hydrogel morphology, as visualized via confocal microscopy in FIGS. 5A-D. As can be seen, neat (PMN-free) pHEMA hydrogels show a porous structure at the surface with cavities ranging from 3 μm up to 15 μm in size, while PMN-incorporating hydrogels display a smooth and cavity-free surface. These morphological differences are also visible in the opacity of PMN-pHEMA hydrogels in contrast with the clear neat pHEMA materials.

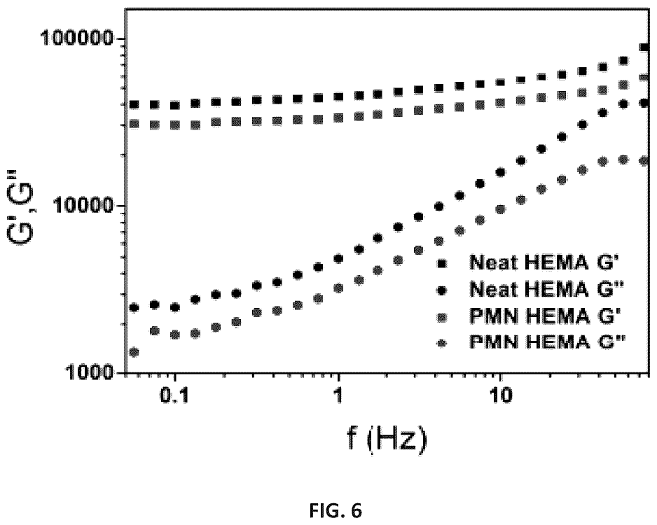

The mechanical properties of the hydrogels were measured and determined rheometrically, and the results are presented in FIG. 6. The obtained data suggest that the hydrogel's storage modulus G′ varies by up to 50% within a frequency (f) range of 0.1 to 10 Hz, and varies between PMN-free and PMN-incorporating hydrogels (2% cross-linked hydrogel). The loss modulus (G″) of the hydrogels also varies within the frequency range of 0.1 to 10 Hz, and between the tested PMN-free and PMN-incorporating hydrogels.

The sliding friction Fs between the hydrogel and a polished stainless-steel surface, as well as surfaces of other materials, was examined over a range of loads Fn. The coefficient of sliding friction, μ, was calculated using the equation:

μ=Fs/Fn

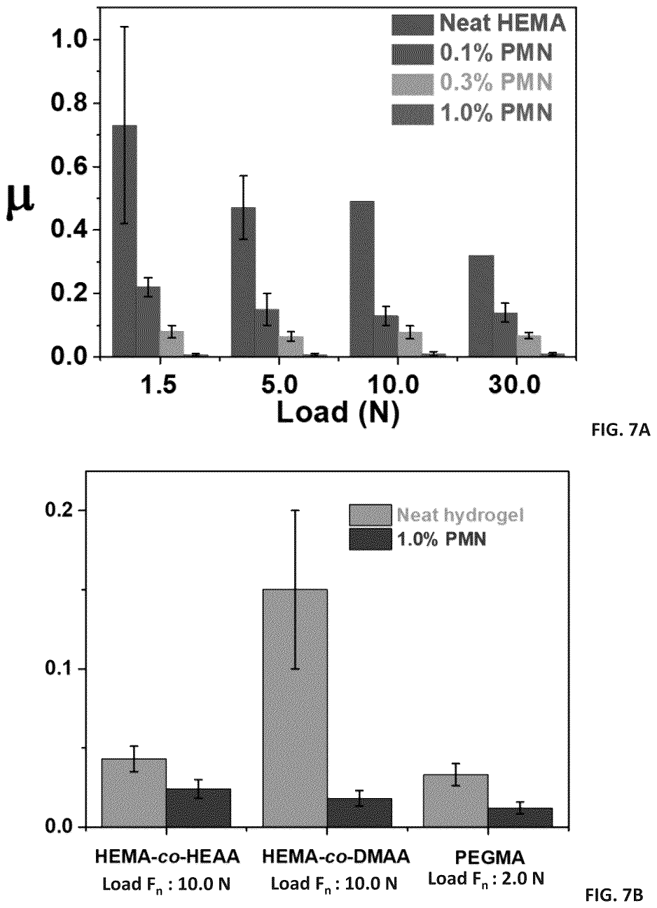

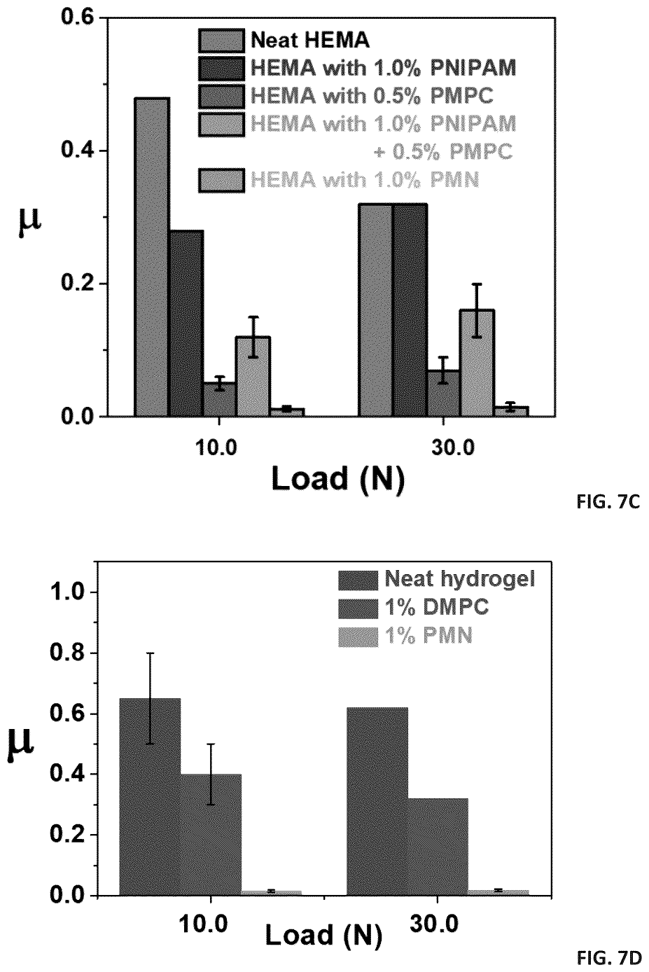

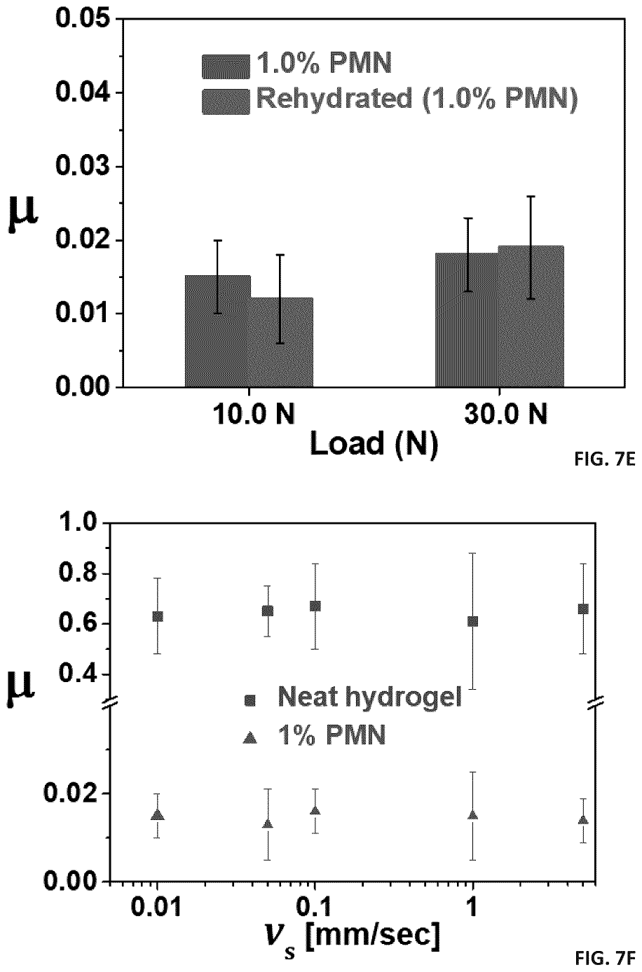

The friction coefficients (μ) of different concentration of PMN (from 0 to 1%) in pHEMA hydrogels were measured at varying loads (1.5 to 30.0 N) against a sliding stainless steel sphere (hydrophilic), and the results are presented in FIG. 7A. A reduction in the friction coefficient can be observed for the PMN-based hydrogels relative to the PMN-free hydrogels (FIG. 7A). For 1% PMN incorporating pHEMA hydrogel, which provides a significantly lower friction coefficient compared to other tested compositions, the reduction in friction coefficient ranges from 95% to 99% at different loads.

The effect of other PMN-incorporating hydrogels on the friction coefficient was then examined in comparison with the respective neat hydrogel, each against a hydrophilic stainless-steel sphere. The results are presented in FIG. 7B and show a reduction in friction coefficient of each hydrogel in respect to its corresponding 1% PMN-incorporating hydrogel.

The effect of incorporating different concentrations of 0.5% pMPC and/or 1% pNIPAM to pHEMA hydrogel on the friction coefficient was tested in comparison with neat pHEMA and with 1% PMN-incorporating hydrogels, each against a hydrophilic stainless-steel sphere. As can be seen in FIG. 7C, all polymer-incorporating hydrogels demonstrated lower friction coefficient. As can be seen, while the combination of pMPC and pNIPAM in the hydrogel resulted in a friction coefficient of about 0.1-0.2, the exemplary p (MPC-co-NIPAM) (PMN)-incorporating hydrogel provided the lowest measured friction coefficient (about 10-fold lower friction coefficient of the hydrogel). Therefore, it can be concluded that compared to different homopolymers-incorporating hydrogel, PMN-incorporating hydrogel showed a strong synergistic effect.

The effect of measuring the friction coefficient against a hydrophobic surface was then examined. For this purpose, the friction coefficients of the PMN-incorporating hydrogel was compared to that of a DMPC lipid-incorporating hydrogel, which was previously suggested to possess low adsorption on hydrophobic surface. As can be seen in FIG. 7D, PMN has significantly reduced the friction coefficient of the hydrogel, while DMPC lowered it to a certain extent.

FIG. 7E shows the effect of a dehydration/rehydration cycle on the friction coefficient of the exemplary PMN-incorporating pHEMA hydrogel, before and after rehydration. It can be observed that the dehydration/rehydration cycle did not significantly affect the friction coefficients of the hydrogel. Indeed, these data demonstrate the ability of these compositions to retain their low friction coefficient after a dehydration/rehydration cycle.

Sliding friction coefficients of neat and PMN-incorporating pHEMA hydrogels were measured at different sliding velocity (Vs), and the results are presented in FIG. 7F. As can be seen, while the friction coefficient of the neat hydrogel is about 0.65, the PMN-incorporating hydrogel's coefficient is about 0.015 (about 40-fold lower).

Hydrophobic counter surfaces were further examined for additional potential application, while comparing the PMN-incorporating hydrogel and the DMPC lipid-incorporating hydrogel. For this purpose, samples were tested by sliding back-and-forth under a 10 N load against a polyethylene sphere for a period of 1 hour.

The results are presented in FIGS. 8A-C, and show that while both the neat (FIG. 8A) and the DMPC-incorporating hydrogels (FIG. 8B) were torn after 5 minutes of back-and-forth shear under the load, the PMB-incorporating hydrogels (FIG. 8C) remained unchanged. These results suggest that the incorporated PMN reduces friction as well as wear and surface damage.

The observed differences in friction between lipid incorporating- and PMN-incorporating hydrogels rubbed by a polyethylene countersurface were assumed to be due to the poor transfer of lipids towards a hydrophobic surface upon sliding.

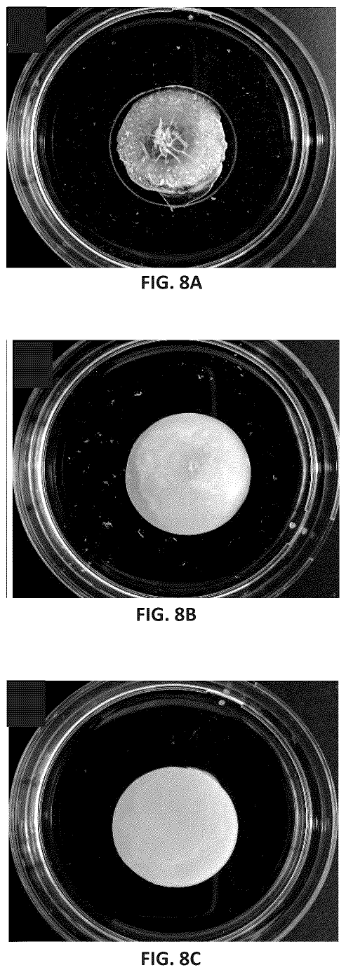

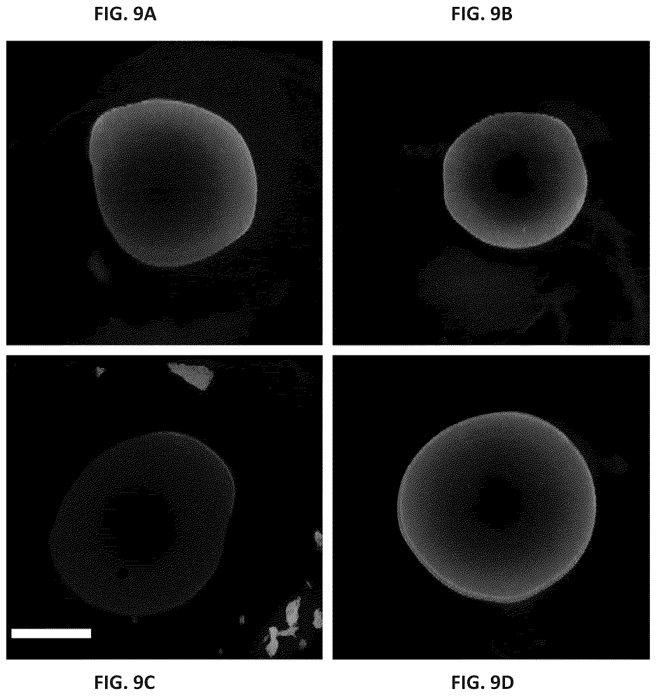

Indeed, comparison of fluorescence signal measured on PE surfaces upon rubbing (FIGS. 9A and 9C) shows a uniform signal coverage for PMN-incorporating hydrogels, indicating efficient transfer of the fluorescently labelled PMN polymer to the surfaces (FIG. 9A). Contrariwise, fluorescence signal on PE surfaces upon rubbing with DMPC-incorporating hydrogels shows weak signal with disrobed fragments of hydrogel (FIG. 9C), in good agreement with the low-friction data.

Replacing the countersurface from hydrophobic PE to the hydrophilic stainless steel (SS) shows uniform fluorescence intensity left on the rubbing surface for both PMN-incorporating and the DMPC-incorporating hydrogels, suggesting efficient transfer for both formulations (FIGS. 9B and 9D).

The amount of transferred material to the ball was further evaluated using fluorescence intensity calibration curves (FIGS. 10A-B).

Table 1 below summarizes adsorption values measured from either SS ball or PE ball upon rubbing for 5 minutes with DMPC or PMN hydrogels. Values are presented as an average and standard deviation from at least three separate samples.

TABLE 1

| | SS ball | PE ball |
|---|---|---|
| PMN | Concentration: 2.20E−3 ± | Concentration: 2.17E−3 ± |
| | 1.888E−5 [mg/mL] | 4.933E−5 [mg/mL] |
| | Area: 0.147 cm² | Area: 0.1 cm² |
| DMPC | Concentration: 2.34E−6 ± | Concentration: 9.60E−7 ± |
| | 8.996E−7 [mg/mL] | 3.44663E−7 [mg/mL] |
| | Area: 0.166 cm² | Area: 0.18 cm² |

The quantification data presented on Table 1 show that PMN-hydrogels deposit a similar amount of rhodamine-labelled PMN both in PE (hydrophobic) and stainless-steel (hydrophilic) surfaces upon rubbing.

DMPC-hydrogels show deposition of DiI-labelled DMPC towards stainless surfaces, in agreement with what reported by Lin et al. (2020, supra) whereas rubbing against PE ball yielded 5 times lower amount of transferred lipids. However, in case of DMPC-hydrogels rubbing against PE surfaces, a significant variability in the estimated deposited amount was observed, possibly due to the presence of fluorescently labelled hydrogel fragments (as visible in FIGS. 9A-D) affecting the overall fluorescence intensity of the recovered material.

These results indicate that PMN-incorporating hydrogels are significantly less affected by the hydrophobicity and/or hydrophilicity of its sliding countersurface, as it performs similarly in terms of lubrication and material deposition with two distinctly different materials (PE and SS).

This feature makes PMN-incorporating hydrogels more adaptable towards multiple applications compared to lipid-incorporating hydrogels for lubrication, which are, as showed, limited in their lubricious properties towards hydrophobic surfaces.

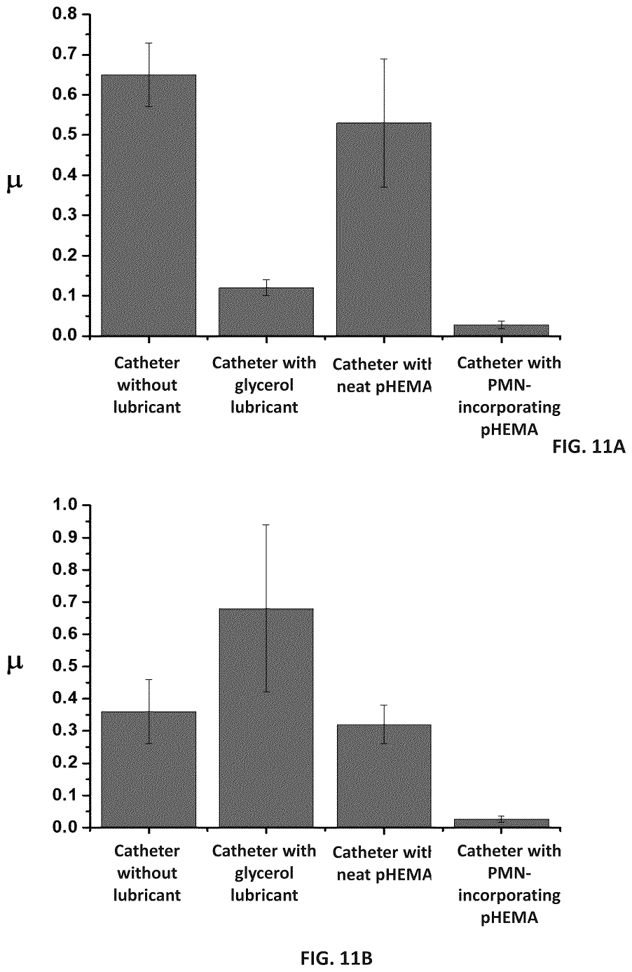

In order to examine the lubrication of a catheter with the PMN-incorporating pHEMA hydrogel in comparison with a catheter with a commercial lubricant (glycerol), a hydrophobic commercialized polyethylene (PE) catheter was used and its sliding friction coefficients (μ) at 100 grams load were tested against a polyethylene (PE) sphere (FIG. 11A) and against a sliding pHEMA sphere (FIG. 11B). As a control, catheters without lubrication and catheters with neat pHEMA were tested as well.

As can be seen in FIG. 11A, the PMN-incorporating pHEMA hydrogel coating shows 80-90% reduction in friction against polyethylene sphere compared to the commercial lubricant.

As FIG. 11B shows, coating the catheter with PMN-incorporating pHEMA reduces the friction by up to 15-folds compared with the best available commercially-lubricated urethral catheter.

### Example 3

### Biocompatibility and Antifouling Properties

Assays for evaluating the cytotoxicity of the hydrogel were performed.

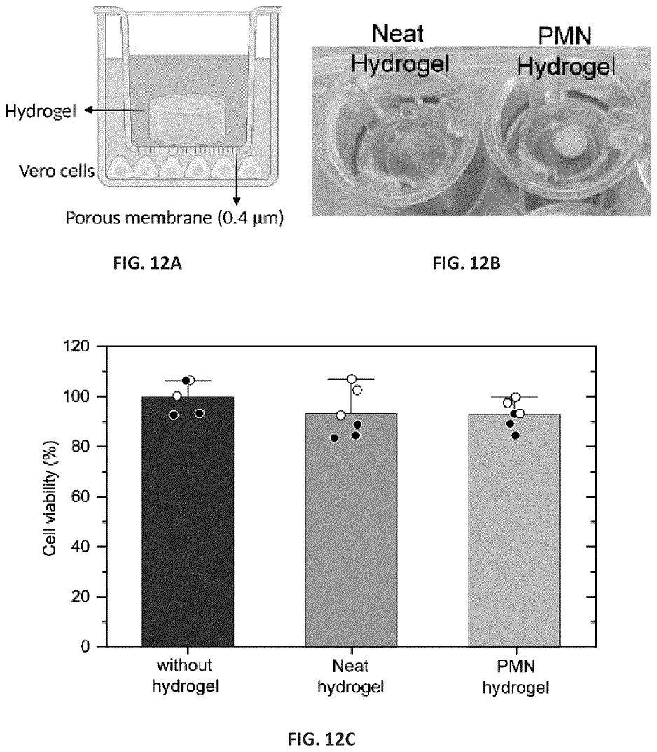

For this purpose, monolayers of Vero cells were exposed to either a PMN-free or a PMN-incorporating pHEMA hydrogels for 48 hours in a Transwell® system (FIGS. 12A-B).

Viability of the cells following 48 hours incubation without hydrogel and with the neat or the PMN-incorporated pHEMA hydrogels was measured, and the results are presented in FIG. 12C. As can be seen, both PMN-free and PMN-incorporating pHEMA hydrogels had no negative impacts on cell viability, with more than 80% cell survival upon exposure to hydrogels, indicating low cytotoxicity (according to ISO Standard 109993-5).

These results demonstrate the capability of PMN-incorporating hydrogels to serve as biomaterials for, e.g., healthcare applications.

The antifouling properties of PMN-incorporating pHEMA hydrogels were evaluated by probing both anti cell-adhesion and anti-bacteria adsorption towards the hydrogels.

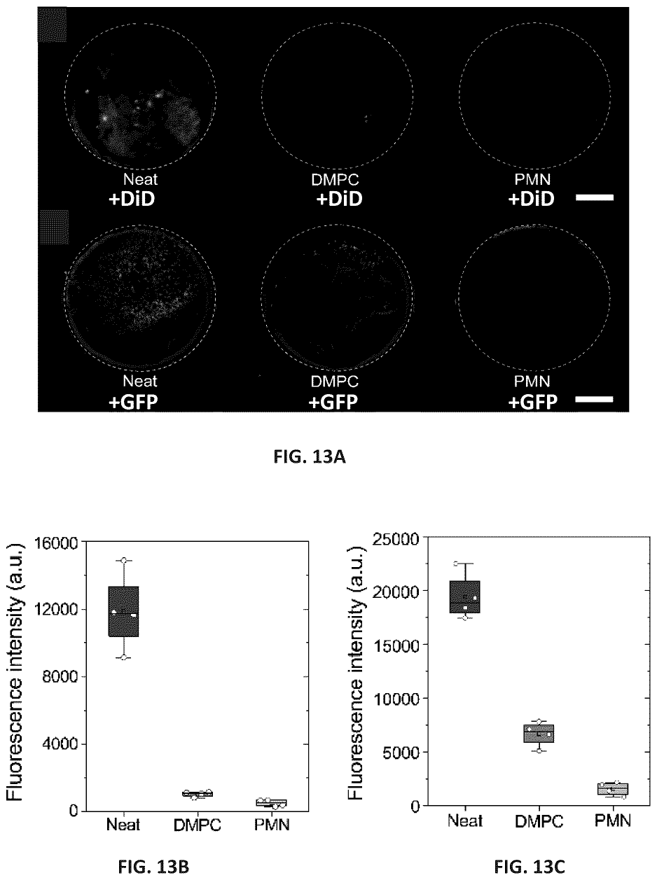

Neat, PMN-incorporating and DMPC-incorporating pHEMA hydrogels were incubated with fluorescently (Di)-labelled Vero cells or GFP-expressing E. coli bacteria suspensions for 24 hours and 12 hours, respectively. The hydrogels were then gently washed. FIG. 13A presents fluorescence images of the hydrogels, and FIGS. 13B-C present the fluorescence quantification, using a plate reader, of DiD fluorescence intensity (FIG. 13B) and of GFP fluorescence intensity (FIG. 13C).

As can be seen, following incubation with Vero cells, a dense population of living cells was observed for neat pHEMA hydrogels 24 hours after incubation (FIGS. 13A-B), as expected for such hydrogels due to the rough surface that favors cell attachment.

In case of incubation with GFP-expressing bacteria, the fluorescence signal of PMN-hydrogel was 17 orders of magnitude lower compared to neat hydrogels and about 5 times lower than DMPC-hydrogels (FIGS. 13A and 13C). It is further observed that the strongest fluorescence signal for PMN-incorporating hydrogels arises from the borders, where the hydrogel was cut, whereas in neat hydrogels bacteria are present in the middle of the hydrogel (FIG. 13A), suggesting that no bacteria are actually present on the PMN-comprising hydrogel surface.

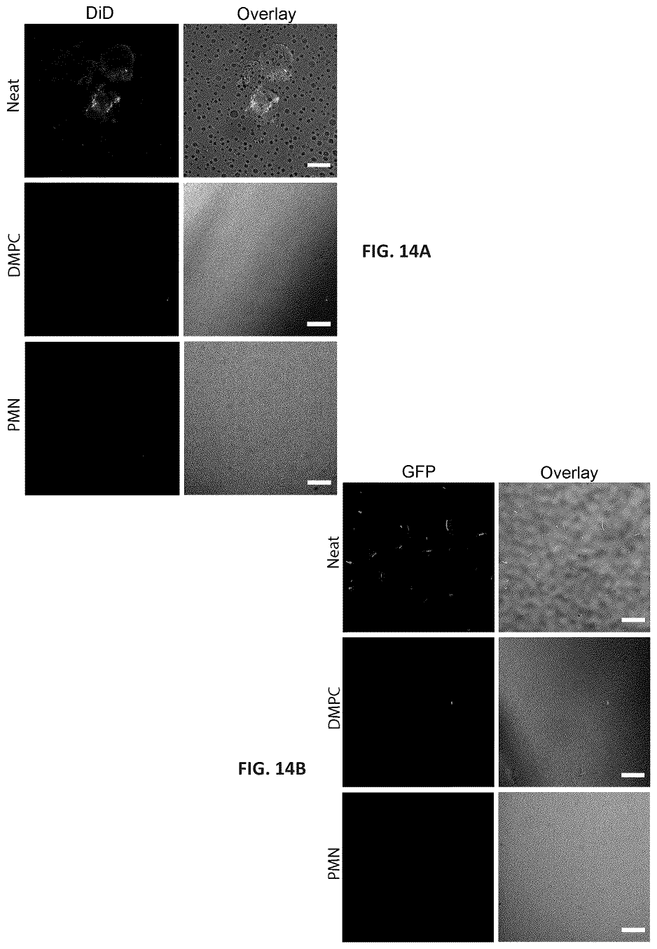

Confocal microscopy visualizations of the tested hydrogels after 24 hours incubation with Vero E6 cells labeled with Vybrant DiD, and after 12 hours incubation with GFP-expressing E. coli bacteria, are presented in FIGS. 14A-B, respectively.

For both DMPC- and PMN-incorporating hydrogels, no presence of cells was observed in confocal microscopy, and a significantly lower fluorescence intensity was obtained compared to pHEMA-only hydrogels, with fluorescence intensity of 1000 a.u. or higher.

These results indicate that the presence of either one of the DMPC lipid and PMN prevents cell adhesion on the hydrogels' surfaces and provides good antifouling properties. Without being bound to any particular theory, because PMN-incorporating hydrogels showed no biocidal action in cell culture assays (FIG. 12C), it is assumed that the observed resistance to cell attachment to hydrogel surface is due to its antifouling characteristic.

Both results with Vero cells and E. coli corroborate that the lubricious nature of PMN-incorporating hydrogels provide desirable antifouling properties, with antibacterial features superior to those of the equally lubricious DMPC-hydrogels.

Overall, incorporating PMN co-polymers within hydrogels significantly reduces the hydrogels' sliding surface friction, resulting in low friction coefficients against different counter surfaces, including stainless steel (hard metal surface), polyethylene (hydrophobic surface), and pHEMA (soft hydrogel surface). In addition, the hydrogels exhibit good anti-bacterial adhesion and anti-cell adsorption.

Hydrogels as reported herein provide a new strategy for various applications (e.g., biomedical applications).

### Example 4

### Effect of N-alkyl Substituents on Lubrication

The effect of the N-alkyl substituents in co-polymeric compound on the lubrication properties of hydrogels containing same was studied. To this end, an N-alkyl analogue of PMN co-polymer, poly(2-methacryloyloxyethyl phosphorylcholine-co-N-propylacrylamide) (PMP) random copolymer was prepared, in which poly(N-propylacrylamide) (nPAM), having N-propylacrylamide substituents, was used instead of NIPAM (having N-isopropylacrylamide substituents).

To assess the effect of the nature of the N-alkyl substituents in the polyacrylamide-based copolymers on lubrication properties of their incorporated hydrogels, poly(2-methacryloyloxyethyl phosphorylcholine-co-N-propylacrylamide) (pMPC-co-pnPAM, PMP) was synthesized using a procedure similar to that for PMN, while replacing NIPAM with N-propylacrylamide (nPAM) monomers. A random PMP co-polymer was prepared according to the following exemplary procedure:

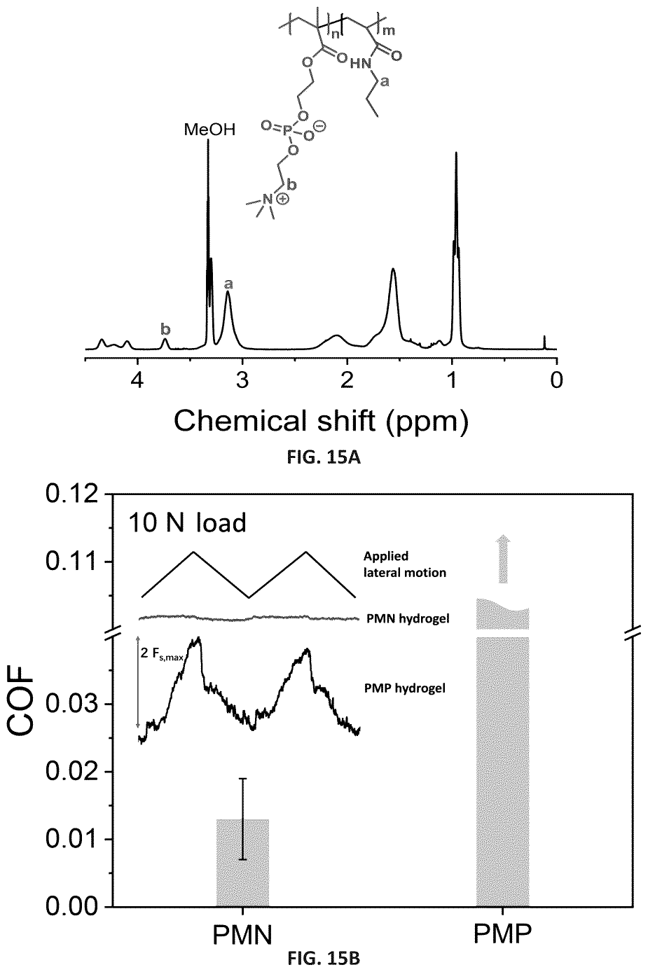

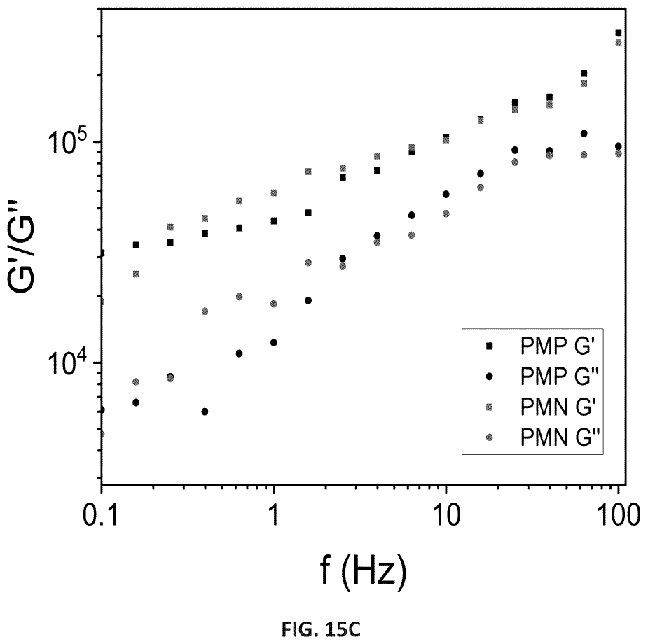

Preparation of a poly(2-methacryloyloxyethyl phosphorylcholine-co-N-propyl acrylamide) random copolymer: MPC (3 grams, 10.2 mmol), nPAM (12 grams, 106 mmol), and APS (320 mg, 1.4 mmol) were dissolved in 200 mL of pure water. The solution was deoxygenated by nitrogen for 30 minutes. After adding 44 μL TEMED, polymerization was carried out at room temperature for 16 hours under a nitrogen atmosphere. The PMP co-polymers were purified by dialysis against pure water for 48 hours. The chemical structure was verified by ¹H NMR (400 MHz, Methanol-d4), a portion of which is presented in FIG. 15A.

As FIG. 15A shows, the characteristic peak (CH₂N) for nPAM is at δ=3.1 ppm, while MPC unit shows the characteristic peak at δ (CH₂N⁺)=3.7 ppm. The estimated ratio of 10:1 between nPAM and MPC was determined by comparing the integral intensity signals.

The PMP co-polymer was incorporated into exemplary pHEMA hydrogels by mixing a low concentration (1% by weight) of PMP with a HEMA solution, as described in further details in the Materials and Methods section hereinabove.

The sliding friction Fs between hydrogels incorporating either PMN-containing or PMP-containing copolymers and a polyethylene (PE) sphere surface (see main text for tribometer measurements description) was examined under load Fn, yielding a friction coefficient (COF, μ=Fs/Fn).

As can be seen in the data on FIG. 15B, for Fn=10 N load, which is about mid-range of the tested loads for a hydrogel incorporating the exemplary co-polymer PMN (see, e.g., FIG. 7A), a COF of more than 0.1 was measured for the PMP-incorporating hydrogels. This COF is greater by an order of magnitude or more than the PMN-incorporating hydrogels, which has a COF of 0.013. The inset of FIG. 15B shows that the mean value for the Fs of PMN co-polymer is 0.015, similar to the previously measured value (see, FIG. 7F). For the PMP co-polymer, no sliding was achieved up to the highest Fs=Fs,max measured (see, FIG. 15B inset, bottom trace), so that the sliding COF is larger than Fs,max/Fn~0.107, as indicated by the curved top of the PMN column in FIG. 15B.

Without being bound to any particular theory, it is assumed that the high COF of the PMP-incorporating hydrogel against PE sphere was obtained due to the linear alkyl moiety of PMP (designated ‘a’ in FIG. 15A), which is more hydrophobic (e.g., based on the Hansch-Fujita Scale) and hence more likely than the branched alkyl moiety (designated ‘a’ in FIG. 1A) (e.g., on the PE countersurface) to form hydrophobic micro-domains. The presence of such domains at the surface of the hydrogel would interact more strongly with the hydrophobic PE countersurface, leading to large dissipation, and hence friction, during sliding.

The mechanical properties of the hydrogels were measured and determined rheometrically, and the results are presented in FIG. 15C. As can be seen, PMP- and PMN-incorporated hydrogels also showed to have similar storage modulus G′ that varies within a frequency (f) in a range of from 0.1 to 100 Hz, and is much larger than G″, the loss modulus.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A co-polymeric compound represented by Formula I: 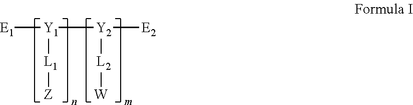
wherein:
m and n are each independently a positive integer;
Y₁ and Y₂ are each independently a backbone unit which forms a polymeric backbone;
L₁ and L₂ are each independently absent or is a linking moiety;
E₁ and E₂ each independently represents a terminal group;
W is a hydrophobic group; and
Z is represented by Formula II: 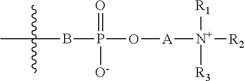
wherein:
A is a substituted or unsubstituted hydrocarbon;
B is an oxygen atom or is absent; and
R₁-R₃ are each independently selected from the group consisting of hydrogen, alkyl, cycloalkyl, heteroalicyclic, aryl and heteroaryl,
wherein when n+m is greater than 2, said —[Y₁-L₁-Z]— units and said —[Y₂-L₂-W]— units are arranged in any order so as to form a random co-polymeric compound, and
wherein a ratio of said n and said m, representing a mol ratio between said —[Y₁-L₁-Z]— units and said —[Y₂-L₂-W]— units in the co-polymeric compound, is in a range of from 1:2 to 1:50 (n:m).

2. The co-polymeric compound of claim 1, wherein Y₁ and Y₂ are each independently a substituted or unsubstituted alkylene unit.

3. The co-polymeric compound of claim 1, wherein:
L₁ is a substituted or unsubstituted hydrocarbon from 1 to 10 carbon atoms in length; and/or
B is an oxygen atom; and/or
A is a substituted or unsubstituted hydrocarbon of from 1 to 4 carbon atoms in length.

4. The co-polymeric compound of claim 1, wherein R₁-R₃ are each independently hydrogen or a C₁₋₄-alkyl.

5. The co-polymeric compound of claim 1, wherein E₁ is or comprises a lipid moiety.

6. The co-polymeric compound of claim 1, wherein the sum of said n and said m is at least 3, or is in a range of from 3 to 5000.

7. The co-polymeric compound of claim 1, wherein:
the percentage of backbone units represented by the variable Y₁ which are substituted by the pendant group represented by (-L₁-Z) (as represented by the formula 100%*n/(n+m), the percentage of variable n from the sum of n+m) is in a range of from 5% to 25%; and/or
wherein the percentage of backbone units represented by the variable Y₂ which are substituted by the pendant group represented by (-L₂-W) (as represented by the formula 100%*m/(n+m), the percentage of variable m from the sum of n+m) is in a range of from 60% to 99%.

8. The co-polymeric compound of claim 1, wherein a ratio of said n and said m, representing a mol ratio between said —[Y₁-L₁-Z]— units and said —[Y₂-L₂-W]— units in the co-polymeric compound, is in a range of from 1:3 to 1:10 (n:m).

9. The co-polymeric compound of claim 1, wherein at least a portion of said Y₁, said Y₂, said E₁, said E₂, said L₁, said L₂, said Z and/or said W comprise a targeting moiety.

10. The co-polymeric compound of claim 1, wherein W is or comprises a branched alkyl of at least 3 carbon atoms in length.

11. The co-polymeric compound of claim 1, wherein W is isopropyl.

12. A composition comprising a hydrogel or a composite material containing a hydrogel, said hydrogel having incorporated therein the co-polymeric compound of claim 1.

13. The composition or composite material of claim 12, wherein an amount of the co-polymeric compound is in a range of from 0.1% to 10%, or from 0.1% to 5%, by weight, of the total weight of the composition.

14. The composition of composite material of claim 12, characterized by at least one of:
Storage modulus G′ (at 1 Hz) of at least 10 MPa;
Loss modulus G″ (at 1 Hz) of at least 1,000 Pa;
Sliding friction coefficient μ lower than 0.05, or lower than 0.01, on a polyethylene (hydrophobic) sphere.

15. A process for preparing the composition or composite of claim 12, the process comprising:
contacting an aqueous solution or suspension comprising a hydrogel-forming agent and a crosslinking agent with the co-polymeric compound, thereby obtaining the composition.

16. A method of lowering a friction coefficient of a hydrogel or of a composite material containing a hydrogel, the method comprising forming the hydrogel in the presence of the co-polymeric compound according to claim 1.

17. An article-of-manufacturing comprising the composition or composite material of claim 12.

18. A method of lowering the friction coefficient of a surface of a substrate, the method comprising applying to the surface the composition or composite material of claim 12.

19. The method of claim 18, wherein said substrate is a physiological substrate or a medical device.

20. A method of inhibiting biofilm formation on a surface of a substrate, the method comprising contacting the substrate with a composition or composite material according to claim 12.
